SEQUENCE LISTING (196 sequences)

SEQ ID NO: 1 - DNA, 369 bp, synthetic construct
  source 1..369: mol_type = other DNA
caggtgcagctggtggagtctgggggaggcgtggtccagcctgggaggtccctgagactctcctgtgcagcctctggattcaccttcagtagttatggcatgcactgggtccgccaggctccaggcaaggggctggagtgggtggcagttatatcatatgatggaagtaataaatactatgcagactccgtgaagggccgattcaccatttccagagacaattccaagaacacgctgtatctgcaaatgaacagcctgagagctgaggacacggctgtgtattactgtgcgaaagtgggagctactactttctactactactacggtatggacgtctggggccaagggaccacggtcaccgtctcctca

SEQ ID NO: 2 - protein, 123 aa, synthetic construct
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCAKVGATTFYYYYGMDVWGQGTTVTVSS

SEQ ID NO: 3 - DNA, 24 bp, synthetic construct
  source 1..24: mol_type = other DNA
ggattcaccttcagtagttatggc

SEQ ID NO: 4 - protein, 8 aa, synthetic construct
GFTFSSYG

SEQ ID NO: 5 - DNA, 24 bp, synthetic construct
  source 1..24: mol_type = other DNA
atatcatatgatggaagtaataaa

SEQ ID NO: 6 - protein, 8 aa, synthetic construct
ISYDGSNK

SEQ ID NO: 7 - DNA, 48 bp, synthetic construct
  source 1..48: mol_type = other DNA
gcgaaagtgggagctactactttctactactactacggtatggacgtc

SEQ ID NO: 8 - protein, 16 aa, synthetic construct
AKVGATTFYYYYGMDV

SEQ ID NO: 9 - DNA, 321 bp, synthetic construct
  source 1..321: mol_type = other DNA
gacatccagatgacccagtctccatcttccgtgtctgcatttgtaggagacagagtcaccatcacttgtcgggcgagtcagggtattagcagctggttagcctggtatcagcagaaaccagggaaagcccctaagctcctgatctatgctgcatccagtttgcaaagtggggtcccatcaaggttcagcggcagtggatctgggacagatttcactctcaccatcagcagcctgcagcctgaagattttgcaacttactattgtcaaaaggctaacagtttcccattcactttcggccctgggaccaaagtggatatcaaa

SEQ ID NO: 10 - protein, 107 aa, synthetic construct
DIQMTQSPSSVSAFVGDRVTITCRASQGISSWLAWYQQKPGKAPKLLIYAASSLQSGVPSRFSGSGSGTDFTLTISSLQPEDFATYYCQKANSFPFTFGPGTKVEIK

SEQ ID NO: 11 - DNA, 18 bp, synthetic construct
  source 1..18: mol_type = other DNA
cagggtattagcagctgg

SEQ ID NO: 12 - protein, 6 aa, synthetic construct
QGISSW

SEQ ID NO: 13-14 - skipped (2 entries)

SEQ ID NO: 15 - DNA, 27 bp, synthetic construct
  source 1..27: mol_type = other DNA
caaaaggctaacagtttcccattcact

SEQ ID NO: 16 - protein, 9 aa, synthetic construct
QKANSFPFT

SEQ ID NO: 17 - DNA, 369 bp, synthetic construct
  source 1..369: mol_type = other DNA
caggtgcagctggtggagtctgggggaggcgtggtccagcctgggaggtccctgagactctcctgtgcagcctctggattcaccttcagtagctatggcatgcactgggtccgccaggctccaggcaaggggctggagtgggtggcagttatatcaaatgatggaagtaataaatactatgtagattccgtgaagggccgattcaccatgggcagagacaattccaagaacacgctgtatctccaaatgaacagcctgagagttgaggacacggctgtgtattactgtgcgaaaggggctggaactctttactactactactacggtatggacgtctggggccaagggaccacggtcaccgtctcctca

SEQ ID NO: 18 - protein, 123 aa, synthetic construct
QVQLVESGGGVVQPGRSLRLSCAASGFTFSSYGMHWVRQAPGKGLEWVAVISNDGSNKYYVDSVKGRFTMGRDNSKNTLYLQMNSLRVEDTAVYYCAKGAGTLYYYYYGMDVWGQGTTVTVSS

SEQ ID NO: 19 - DNA, 24 bp, synthetic construct
  source 1..24: mol_type = other DNA
ggattcaccttcagtagctatggc

SEQ ID NO: 20 - protein, 8 aa, synthetic construct
GFTFSSYG

SEQ ID NO: 21 - DNA, 24 bp, synthetic construct
  source 1..24: mol_type = other DNA
atatcaaatgatggaagtaataaa

SEQ ID NO: 22 - protein, 8 aa, synthetic construct
ISNDGSNK

SEQ ID NO: 23 - DNA, 48 bp, synthetic construct
  source 1..48: mol_type = other DNA
gcgaaaggggctggaactctttactactactactacggtatggacgtc

SEQ ID NO: 24 - protein, 16 aa, synthetic construct
AKGAGTLYYYYYGMDV

SEQ ID NO: 25 - DNA, 321 bp, synthetic construct
  source 1..321: mol_type = other DNA
gacatccagatgacccagtctccttccaccctgtctgcatctgtaggaaacagagtcaccatcacttgccgggccagtcaaagtattagtagctggttggcctggtatcaacaaaaaccagggaaagcccctaagttcctgatctataaggcgtctagtttagaaagtggggtcccatcaaggttcagcggcagtggatctgggacagaattcactctcaccatcaccagcctgcagcctgatgattttgcaacttattactgccaacagtacaatatttattcgtggacgttcggccaagggaccaaggtggaaatcaaa

SEQ ID NO: 26 - protein, 107 aa, synthetic construct
DIQMTQSPSTLSASVGNRVTITCRASQSISSWLAWYQQKPGKAPKFLIYKASSLESGVPSRFSGSGSGTEFTLTITSLQPDDFATYYCQQYNIYSWTFGQGTKVEIK

SEQ ID NO: 27 - DNA, 18 bp, synthetic construct
  source 1..18: mol_type = other DNA
caaagtattagtagctgg

SEQ ID NO: 28 - protein, 6 aa, synthetic construct
QSISSW

SEQ ID NO: 29-30 - skipped (2 entries)

SEQ ID NO: 31 - DNA, 27 bp, synthetic construct
  source 1..27: mol_type = other DNA
caacagtacaatatttattcgtggacg

SEQ ID NO: 32 - protein, 9 aa, synthetic construct
QQYNIYSWT

SEQ ID NO: 33 - DNA, 372 bp, synthetic construct
  source 1..372: mol_type = other DNA
caggtgcagctggtggagtctgggggaggcgtggtccagcctgggaggtccctgagactctcctgtgcagcctctggattcaccttcagtacctatggcatgcactgggtccgccaggctccaggcaaggggctggagtggatggcagttatatcatttgatagaggtaataaatactatgcagactccgtgaagggccgattcaccatctccagagacaattccaagaacacgctgtatctgcaaatgaacagcctgagagctgaggacacggctgtgtattactgtgcgaaaggggggggttcggggactttctactactactacggtatggacgtctggggccaagggaccacggtcaccgtctcctca

SEQ ID NO: 34 - protein, 124 aa, synthetic construct
QVQLVQSGGGVVQPGRSLRLSCAASGFTFSTYGMHWVRQAPGKGLEWMAVISFDRGNKYYADSVKGRFTISRDNSKNTLYLQMNSLRAEDTAVYYCAKGGGSGTFYYYYGMDVWGQGTTVTVSS

SEQ ID NO: 35 - DNA, 24 bp, synthetic construct
  source 1..24: mol_type = other DNA
ggattcaccttcagtacctatggc

SEQ ID NO: 36 - protein, 8 aa, synthetic construct
GFTFSTYG

SEQ ID NO: 37 - DNA, 24 bp, synthetic construct
  source 1..24: mol_type = other DNA
atatcatttgatagaggtaataaa

SEQ ID NO: 38 - protein, 8 aa, synthetic construct
ISFDRGNK

SEQ ID NO: 39 - DNA, 51 bp, synthetic construct
  source 1..51: mol_type = other DNA
gcgaaaggggggggttcggggactttctactactactacggtatggacgtc

SEQ ID NO: 40 - protein, 17 aa, synthetic construct
AKGGGSGTFYYYYGMDV

SEQ ID NO: 41 - DNA, 324 bp, synthetic construct
  source 1..324: mol_type = other DNA
gacatccagatgacccagtctccatcttccgtgtctgcatctgtaggaaacagagtcaccatcacttgtcgggcgagtcagggtattagcagctggttagcctggtatcaccagaaaccagggaaagtccctaaggtcctgatctatgctgcatccagtttgcaaagtggggtcccatcaaggttcagcggcagtggatctgggacagatttcactctcaccatcagcagcctgcagcctgaagattttgcaacttactattgtcaacaggctaacagtttcccattcactttcggccctgggaccaaagtggatatcaaacga

SEQ ID NO: 42 - protein, 108 aa, synthetic construct
DIQMTQSPSSVSASVGNRVTITCRASQGISSWLAWYHQKPGKVPKVLIYAASSLQSGVPSRFSGSGSGTDFTLTISSLQPEDFATYYCQQANSFPFTFGPGTKVEIKR

SEQ ID NO: 43 - DNA, 18 bp, synthetic construct
  source 1..18: mol_type = other DNA
cagggtattagcagctgg

SEQ ID NO: 44 - protein, 6 aa, synthetic construct
QGISSW

SEQ ID NO: 45-46 - skipped (2 entries)

SEQ ID NO: 47 - DNA, 27 bp, synthetic construct
  source 1..27: mol_type = other DNA
caacaggctaacagtttcccattcact

SEQ ID NO: 48 - protein, 9 aa, synthetic construct
QQANSFPFT

SEQ ID NO: 49 - DNA, 375 bp, synthetic construct
  source 1..375: mol_type = other DNA
caggtacagctgcagcagtcaggtccaggactggtgaaaccctcgcagaccctctcactcacctgtgccatctccggggacagtgtctctagcaacagtcctgcttggaactggatcaggcagtccccatcgagaggccttgagtggctgggaaggacatactacaggtccaagtggtataatgattatgcagtgtctgtgagaggtcgaataaccatcaacccagacacatccaataaccagttctccctacatctgaactctgtgactcccgaggacacggcgatgtattactgtgcaagagacaagggtctaacagctcgtccgacctactttgactactggggccagggaaccctggtcaccgtctcctca

SEQ ID NO: 50 - protein, 125 aa, synthetic construct
QVQLQQSGPGLVKPSQTLSLTCAISGDSVSSNSPAWNWIRQSPSRGLEWLGRTYYRSKWYNDYAVSVRGRITINPDTSNNQFSLHLNSVTPEDTAMYYCARDKGLTARPTYFDYWGQGTLVTVSS

SEQ ID NO: 51 - DNA, 30 bp, synthetic construct
  source 1..30: mol_type = other DNA
ggggacagtgtctctagcaacagtcctgct

SEQ ID NO: 52 - protein, 10 aa, synthetic construct
GDSVSSNSPA

SEQ ID NO: 53 - DNA, 27 bp, synthetic construct
  source 1..27: mol_type = other DNA
acatactacaggtccaagtggtataat

SEQ ID NO: 54 - protein, 9 aa, synthetic construct
TYYRSKWYN

SEQ ID NO: 55 - DNA, 45 bp, synthetic construct
  source 1..45: mol_type = other DNA
gcaagagacaagggtctaacagctcgtccgacctactttgactac

SEQ ID NO: 56 - protein, 15 aa, synthetic construct
ARDKGLTARPTYFDY

SEQ ID NO: 57 - DNA, 326 bp, synthetic construct
  source 1..326: mol_type = other DNA
gacatccagatgacccagtctccttccaccctgtctgcatctgtaggagacagagtcaccatcacttgccgggccagtcagagtattaattactggttggcctggtatcagcagaaaccagggaaagcccctaagccccttatctataaggcgtctagtttagaaagtggggtcccatcaaggttcagcggcagtggatctgggacagagttcactctcaccatcagcagcctgcagcctgacgattttacaacttattactgccaacagtataatagttattctccgacgttcggccaagggaccaaggtggaaatcaaacgaac

SEQ ID NO: 58 - protein, 108 aa, synthetic construct
DIQMTQSPSTLSASVGDRVTITCRASQSINYWLAWYQQKPGKAPKPLIYKASSLESGVPSRFSGSGSGTEFTLTISSLQPDDFTTYYCQQYNSYSPTFGQGTKLEIKR

SEQ ID NO: 59 - DNA, 18 bp, synthetic construct
  source 1..18: mol_type = other DNA
cagagtattaattactgg

SEQ ID NO: 60 - protein, 6 aa, synthetic construct
QSINYW

SEQ ID NO: 61-62 - skipped (2 entries)

SEQ ID NO: 63 - DNA, 27 bp, synthetic construct
  source 1..27: mol_type = other DNA
caacagtataatagttattctccgacg

SEQ ID NO: 64 - protein, 9 aa, synthetic construct
QQYNSYSPT

SEQ ID NO: 65 - DNA, 378 bp, synthetic construct
  source 1..378: mol_type = other DNA
gaggtgcagctggtggagtctgggggaggcgtgatacagcctggggggtccctgagactctcctgtgcagcctctggattcaccttcgatgattatgccatgaactgggtccgtcaaggtccagggaagggtctggagtgggtctctgccataagtggtgatggcggtagcacatactatgcagactcggtgaagggccgattcaccatctccagagacaacagcaaaaactccctgtatctgcaaatgaacagtctgagagctgaggacaccgcctttttttactgtgcaaaagatctccgtaatacgatttttggagtggttattcccgatgcttttgatatctggggccaagggacaatggtcaccgtctcttca

SEQ ID NO: 66 - protein, 126 aa, synthetic construct
EVQLVESGGGVIQPGGSLRLSCAASGFTFDDYAMNWVRQGPGKGLEWVSAISGDGGSTYYADSVKGRFTISRDNSKNSLYLQMNSLRAEDTAFFYCAKDLRNTIFGVVIPDAFDIWGQGTMVTVSS

SEQ ID NO: 67 - DNA, 24 bp, synthetic construct
  source 1..24: mol_type = other DNA
ggattcaccttcgatgattatgcc

SEQ ID NO: 68 - protein, 8 aa, synthetic construct
GFTFDDYA

SEQ ID NO: 69 - DNA, 24 bp, synthetic construct
  source 1..24: mol_type = other DNA
ataagtggtgatggcggtagcaca

SEQ ID NO: 70 - protein, 8 aa, synthetic construct
ISGDGGST

SEQ ID NO: 71 - DNA, 57 bp, synthetic construct
  source 1..57: mol_type = other DNA
gcaaaagatctccgtaatacgatttttggagtggttattcccgatgcttttgatatc

SEQ ID NO: 72 - protein, 19 aa, synthetic construct
AKDLRNTIFGVVIPDAFDI

SEQ ID NO: 73 - DNA, 324 bp, synthetic construct
  source 1..324: mol_type = other DNA
gacatccagatgacccagtctccttccaccctgtctgcatctgtaggagacagagtcaccatcacttgccgggccagtcagagcattaggagctggttggcctggtatcagcagaaaccagggaaagcccctaaactcctgatctataaggcgtctagtttagaaagtggggtcccatcaaggttcagcggcagtggatctgggacagaattcactctcaccatcagcagcctgcagcctgatgattttgcaacttattactgccaacaatataatagttattcgtacacttttggccaggggaccaagctggagatcaaacga

SEQ ID NO: 74 - protein, 108 aa, synthetic construct
DIQMTQSPSTLSASVGDRVTITCRASQSIRSWLAWYQQKPGKAPKLLIYKASSLESGVPSRFSGSGSGTEFTLTISSLQPDDFATYYCQQYNSYSYTFGQGTKLEIKR

SEQ ID NO: 75 - DNA, 18 bp, synthetic construct
  source 1..18: mol_type = other DNA
cagagcattaggagctgg

SEQ ID NO: 76 - protein, 6 aa, synthetic construct
QSIRSW

SEQ ID NO: 77-78 - skipped (2 entries)

SEQ ID NO: 79 - DNA, 27 bp, synthetic construct
  source 1..27: mol_type = other DNA
caacaatataatagttattcgtacact

SEQ ID NO: 80 - protein, 9 aa, synthetic construct
QQYNSYSYT

SEQ ID NO: 81 - DNA, 354 bp, synthetic construct
  source 1..354: mol_type = other DNA
caggtgcagctggtggagtcggggggaggcttggtcaagcctggagggtccctgagactctcctgtgcagcctctggattcactttcagtgactactacatgagctggatccgtcaggctccagggaaggggctggagtgggtttcatacattggtagtagtggtgtcaacatgtactacgcagactctgtgaagggccgattcaccatctccagggacaacgccaagaattcattatatctggaaatgaacagcctgagagccgaggacacggctgtttattactgtgcgagagactcttcccaactgggttttgactactggggccagggaaccctggtcaccgtctcctca

SEQ ID NO: 82 - protein, 118 aa, synthetic construct
QVQLVESGGGLVKPGGSLRLSCAASGFTFSDYYMSWIRQAPGKGLEWVSYIGSSGVNMYYADSVKGRFTISRDNAKNSLYLEMNSLRAEDTAVYYCARDSSQLGFDYWGQGTLVTVSS

SEQ ID NO: 83 - DNA, 24 bp, synthetic construct
  source 1..24: mol_type = other DNA
ggattcactttcagtgactactac

SEQ ID NO: 84 - protein, 8 aa, synthetic construct
GFTFSDYY

SEQ ID NO: 85 - DNA, 24 bp, synthetic construct
  source 1..24: mol_type = other DNA
attggtagtagtggtgtcaacatg

SEQ ID NO: 86 - protein, 8 aa, synthetic construct
IGSSGVNM

SEQ ID NO: 87 - DNA, 33 bp, synthetic construct
  source 1..33: mol_type = other DNA
gcgagagactcttcccaactgggttttgactac

SEQ ID NO: 88 - protein, 11 aa, synthetic construct
ARDSSQLGFDY

SEQ ID NO: 89 - DNA, 324 bp, synthetic construct
  source 1..324: mol_type = other DNA
gacatccagatgacccagtctccatcctccctgtctgcatctgtaggagacagagtcaccatcacttgccggacaagtcagaatattatcaactttttaaattggtatcaacagaaacctgggaaggcccctaaactcctgatctatactacttccactttacaaagtggggtcccatcaaggttcagtggcagtggatctgggacagatttcactctctccatcaatagtctacaacctgaagattttgcaacttacttctgtcaacagacttacagtaatccactcactttcggcggagggaccaaggtggagatcaaacga

SEQ ID NO: 90 - protein, 108 aa, synthetic construct
DIQMTQSPSSLSASVGDRVTITCRTSQNIINFLNWYQQKPGKAPKLLIYTTSTLQSGVPSRFSGSGSGTDFTLSINSLQPEDFATYFCQQTYSNPLTFGGGTKLEIKR

SEQ ID NO: 91 - DNA, 18 bp, synthetic construct
  source 1..18: mol_type = other DNA
cagaatattatcaacttt

SEQ ID NO: 92 - protein, 6 aa, synthetic construct
QNIINF

SEQ ID NO: 93-94 - skipped (2 entries)

SEQ ID NO: 95 - DNA, 27 bp, synthetic construct
  source 1..27: mol_type = other DNA
caacagacttacagtaatccactcact

SEQ ID NO: 96 - protein, 9 aa, synthetic construct
QQTYSNPLT

SEQ ID NO: 97 - DNA, 360 bp, synthetic construct
  source 1..360: mol_type = other DNA
gaggtgcagctggtgcagtctgggggaggcgtggtccagcctgggaggtccctgagactctcctgtgcagcgtctggattcaccttcagtaattatggcatgcactgggtccgccaggctccaggcaaggggctggagtgggtggcagttgtttggtatgatggagataataaatactatgcagactccgtgaagggccgattcaccatctccagagacaattccaagaacacgctgtatctgcaaatgaacagcctgagagccgaggacacggctgtgtattactgtgcgagagatattatcacatctcgcccgactttggactactggggccagggaaccctggtcactgtctcctca

SEQ ID NO: 98 - protein, 120 aa, synthetic construct
EVQLVQSGGGVVQPGRSLRLSCAASGFTFSNYGMHWVRQAPGKGLEWVAVVWYDGDNKYYADSVKGRFTISRDNSKNTLYLQMNSLRAEDTAVYYCARDIITSRPTLDYWGQGTLVTVSS

SEQ ID NO: 99 - DNA, 24 bp, synthetic construct
  source 1..24: mol_type = other DNA
ggattcaccttcagtaattatggc

SEQ ID NO: 100 - protein, 8 aa, synthetic construct
GFTFSNYG

SEQ ID NO: 101 - DNA, 24 bp, synthetic construct
  source 1..24: mol_type = other DNA
gtttggtatgatggagataataaa

SEQ ID NO: 102 - protein, 8 aa, synthetic construct
VWYDGDNK

SEQ ID NO: 103 - DNA, 39 bp, synthetic construct
  source 1..39: mol_type = other DNA
gcgagagatattatcacatctcgcccgactttggactac

SEQ ID NO: 104 - protein, 13 aa, synthetic construct
ARDIITSRPTLDY

SEQ ID NO: 105 - DNA, 324 bp, synthetic construct
  source 1..324: mol_type = other DNA
gacatccagttgacccagtctccatccttcctgtctgcatctgtaggagacagagtcaccatcacttgctgggccagtcagggcattaacagttatttagcctggtatcagcaaaaaccagggaaagcccctaagctcctgatctatcctgcatccactttgcaaagtggggtcccatcaaggttcagcggcagtggatctgggacagaattcactctcacaatcagcagcctgcagcctgtagattttgcaacttattactgtcaacagcttaatagttacccgctcactttcggcggagggaccaaggtggagatcaaacga

SEQ ID NO: 106 - protein, 108 aa, synthetic construct
DIQLTQSPSFLSASVGDRVTITCWASQGINSYLAWYQQKPGKAPKLLIYPASTLQSGVPSRFSGSGSGTEFTLTISSLQPVDFATYYCQQLNSYPLTFGGGTKVEIKR

SEQ ID NO: 107 - DNA, 18 bp, synthetic construct
  source 1..18: mol_type = other DNA
cagggcattaacagttat

SEQ ID NO: 108 - protein, 6 aa, synthetic construct
QGINSY

SEQ ID NO: 109-110 - skipped (2 entries)

SEQ ID NO: 111 - DNA, 27 bp, synthetic construct
  source 1..27: mol_type = other DNA
caacagcttaatagttacccgctcact

SEQ ID NO: 112 - protein, 9 aa, synthetic construct
QQLNSYPLT

SEQ ID NO: 113 - DNA, 354 bp, synthetic construct
  source 1..354: mol_type = other DNA
caggtgcagctggtggagtcggggggaggcttggtcaagcctggagggtccctgagactctcctgtgcagcctctggattcaccttcagtgactactacatgagctggatccgccaggctccagggaaggggctggagtgggtttcatacattagtagtagtggtagtaccatatactacgcagactctgtgaagggccgattcaccatatccagggacaacgccaagaactcactgtatctgcaaatgaacagcctgagagccgaggacacggccgtgtattactgtgcgagagattcttcccaactgggttttgactactggggccagggaaccctggtcaccgtctcctca

SEQ ID NO: 114 - protein, 118 aa, synthetic construct
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARDSSQLGFDYWGQGTLVTVSS

SEQ ID NO: 115 - DNA, 24 bp, synthetic construct
  source 1..24: mol_type = other DNA
ggattcaccttcagtgactactac

SEQ ID NO: 116 - protein, 8 aa, synthetic construct
GFTFSDYY

SEQ ID NO: 117 - DNA, 24 bp, synthetic construct
  source 1..24: mol_type = other DNA
attagtagtagtggtagtaccata

SEQ ID NO: 118 - protein, 8 aa, synthetic construct
ISSSGSTI

SEQ ID NO: 119 - DNA, 33 bp, synthetic construct
  source 1..33: mol_type = other DNA
gcgagagattcttcccaactgggttttgactac

SEQ ID NO: 120 - protein, 11 aa, synthetic construct
ARDSSQLGFDY

SEQ ID NO: 121 - DNA, 324 bp, synthetic construct
  source 1..324: mol_type = other DNA
gacatccagatgacccagtctccatcctccctgtctgcatctgtaggagacagagtcaccatcacttgccgggcaagtcagagcattatcagctttttaaattggtatcagcagaaaccagggaaggcccctaagctcctgatctatactgcatccagtttgcaaagtggggtcccatcaaggttcagtggcagtggatctgggacagatttcactctcaccatcagcagtctacaacctgaagattttgcaacttactactgtcaacagacttacagtaatccgctcactttcggcggagggaccaaggtggaaatcaaacga

SEQ ID NO: 122 - protein, 108 aa, synthetic construct
DIQMTQSPSSLSASVGDRVTITCRASQSIISFLNWYQQKPGKAPKLLIYTASSLQSGVPSRFSGSGSGTDFTLTISSLQPEDFATYYCQQTYSNPLTFGGGTKVEIKR

SEQ ID NO: 123 - DNA, 18 bp, synthetic construct
  source 1..18: mol_type = other DNA
cagagcattatcagcttt

SEQ ID NO: 124 - protein, 6 aa, synthetic construct
QSIISF

SEQ ID NO: 125-126 - skipped (2 entries)

SEQ ID NO: 127 - DNA, 27 bp, synthetic construct
  source 1..27: mol_type = other DNA
caacagacttacagtaatccgctcact

SEQ ID NO: 128 - protein, 9 aa, synthetic construct
QQTYSNPLT

SEQ ID NO: 129 - DNA, 342 bp, synthetic construct
  source 1..342: mol_type = other DNA
caggtgcagctggtacagtctggacctgaggtgaagaagcctggggcctcagtgagggtctcctgtaaggcttctggttaccttagtgactttattatcacctgggtgcgacaggcccctggacaagggcttgagtggatgggatggatcagcacttacagtggtgacacagactctgcaccgaagttccagggcagagtcaccatgaccacagacacatccacgactacagtcttcttggaactgaggagcctgagatctgacgacacggccgtgtattattgtgcgagagggcggctgtttgactactggggccagggaaccctggtcaccgtctcctca

SEQ ID NO: 130 - protein, 114 aa, synthetic construct
QVQLVQSGPEVKKPGASVRVSCKASGYLSDFIITWVRQAPGQGLEWMGWISTYSGDTDSAPKFQGRVTMTTDTSTTTVFLELRSLRSDDTAVYYCARGRLFDYWGQGTLVTVSS

SEQ ID NO: 131 - DNA, 21 bp, synthetic construct
  source 1..21: mol_type = other DNA
ggttaccttagtgactttatt

SEQ ID NO: 132 - protein, 7 aa, synthetic construct
GYLSDFI

SEQ ID NO: 133 - DNA, 24 bp, synthetic construct
  source 1..24: mol_type = other DNA
atcagcacttacagtggtgacaca

SEQ ID NO: 134 - protein, 8 aa, synthetic construct
ISTYSGDT

SEQ ID NO: 135 - DNA, 24 bp, synthetic construct
  source 1..24: mol_type = other DNA
gcgagagggcggctgtttgactac

SEQ ID NO: 136 - protein, 8 aa, synthetic construct
ARGRLFDY

SEQ ID NO: 137 - DNA, 339 bp, synthetic construct
  source 1..339: mol_type = other DNA
gatgttgtgatgactcagtctccactctccctgcccgtcacccttggacagccggcctccatctcctgcaggtctagtcaaagcctcgtatacagtgatggaaacacctacttgaattggtttcaacagaggccaggccaatctccaaggcgcctaatttataaggtttctaaccgggactctggggtcccagacagattcagcggcagtgggtcaggcactgatttcacactgaaaatcagcagggtggaggctgaggatgttggggtttattactgcatgcaaggtacacactggccgtacacttttggccaggggaccaagctggagatcaaacga

SEQ ID NO: 138 - protein, 113 aa, synthetic construct
DVVMTQSPLSLPVTLGQPASISCRSSQSLVYSDGNTYLNWFQQRPGQSPRRLIYKVSNRDSGVPDRFSGSGSGTDFTLKISRVEAEDVGVYYCMQGTHWPYTFGQGTKLEIKR

SEQ ID NO: 139 - DNA, 33 bp, synthetic construct
  source 1..33: mol_type = other DNA
caaagcctcgtatacagtgatggaaacacctac

SEQ ID NO: 140 - protein, 11 aa, synthetic construct
QSLVYSDGNTY

SEQ ID NO: 141-142 - skipped (2 entries)

SEQ ID NO: 143 - DNA, 27 bp, synthetic construct
  source 1..27: mol_type = other DNA
atgcaaggtacacactggccgtacact

SEQ ID NO: 144 - protein, 9 aa, synthetic construct
MQGTHWPYT

SEQ ID NO: 145 - DNA, 354 bp, synthetic construct
  source 1..354: mol_type = other DNA
caggtgcagctggtggagtctgggggagtctcggtcaagcctggagggtccctgcgactctcctgtgcagcctctggattcaccttcagtgactactacatgagctggatccgccaggcgccagggaagggactggagtgggtttcgtacattggtagtagtggtactaatgactactacgcagactctgtgaagggccgattcaccatctccagggacaacgccaagaactcactgtatcttcaaatggacagcctgagagccgaggacacggccgtctattactgtgcgagagattcttcccaaatgggttttgactactggggccagggaaccctggtcactgtctcctca

SEQ ID NO: 146 - protein, 118 aa, synthetic construct
QVQLVESGGVSVKPGGSLRLSCAASGFTFSDYYMSWIRQAPGKGLEWVSYIGSSGTNDYYADSVKGRFTISRDNAKNSLYLQMDSLRAEDTAVYYCARDSSQMGFDYWGQGTLVTVSS

SEQ ID NO: 147 - DNA, 24 bp, synthetic construct
  source 1..24: mol_type = other DNA
ggattcaccttcagtgactactac

SEQ ID NO: 148 - protein, 8 aa, synthetic construct
GFTFSDYY

SEQ ID NO: 149 - DNA, 24 bp, synthetic construct
  source 1..24: mol_type = other DNA
attggtagtagtggtactaatgac

SEQ ID NO: 150 - protein, 8 aa, synthetic construct
IGSSGTND

SEQ ID NO: 151 - DNA, 33 bp, synthetic construct
  source 1..33: mol_type = other DNA
gcgagagattcttcccaaatgggttttgactac

SEQ ID NO: 152 - protein, 11 aa, synthetic construct
ARDSSQMGFDY

SEQ ID NO: 153 - DNA, 324 bp, synthetic construct
  source 1..324: mol_type = other DNA
gacatccagatgacccagtctccatcctccctgtctgcttctgtgggagacagagtcaccatcacttgccgggcaagtcagaacattatcaactttttaaattggtatcagcagagaccagggaaagcccctcagctcctgatctatgttgcagccagcttgcagagtggggtcccatcaaggttcactggcagtggatatgggacagatttcactctcaccatcagcagtctgcaacctgaggatttcgcaacttactactgtcaacagacttacactaacccgctcactttcggcggagggaccaaggtggagatcaaacga

SEQ ID NO: 154 - protein, 108 aa, synthetic construct
DIQMTQSPSSLSASVGDRVTITCRASQNIINFLNWYQQRPGKAPQLLIYVAASLQSGVPSRFTGSGYGTDFTLTISSLQPEDFATYYCQQTYTNPLTFGGGTKVEIKR

SEQ ID NO: 155 - DNA, 18 bp, synthetic construct
  source 1..18: mol_type = other DNA
cagaacattatcaacttt

SEQ ID NO: 156 - protein, 6 aa, synthetic construct
QNIINF

SEQ ID NO: 157-158 - skipped (2 entries)

SEQ ID NO: 159 - DNA, 27 bp, synthetic construct
  source 1..27: mol_type = other DNA
caacagacttacactaacccgctcact

SEQ ID NO: 160 - protein, 9 aa, synthetic construct
QQTYTNPLT

SEQ ID NO: 161 - protein, 460 aa, Homo sapiens
MFTIKLLLFIVPLVISSRIDQDNSSFDSLSPEPKSRFAMLDDVKILANGLLQLGHGLKDFVHKTKGQINDIFQKLNIFDQSFYDLSLQTSEIKEEEKELRRTTYKLQVKNEEVKNMSLELNSKLESLLEEKILLQQKVKYLEEQLTNLIQNQPETPEHPEVTSLKTFVEKQDNSIKDLLQTVEDQYKQLNQQHSQIKEIENQLRRTSIQEPTEISLSSKPRAPRTTPFLQLNEIRNVKHDGIPAECTTIYNRGEHTSGMYAIRPSNSQVFHVYCDVISGSPWTLIQHRIDGSQNFNETWENYKYGFGRLDGEFWLGLEKIYSIVKQSNYVLRIELEDWKDNKHYIEYSFYLGNHETNYTLHLVAITGNVPNAIPENKDLVFSTWDHKAKGHFNCPEGYSGGWWWHDECGENNLNGKYNKPRAKSKPERRRGLSWKSQNGRLYSIKSTKMLIHPTDSESFE

SEQ ID NO: 162 - DNA, 1383 bp, Homo sapiens
  source 1..1383: mol_type = unassigned DNA
atgttcacaattaagctccttctttttattgttcctctagttatttcctccagaattgatcaagacaattcatcatttgattctctatctccagagccaaaatcaagatttgctatgttagacgatgtaaaaattttagccaatggcctccttcagttgggacatggtcttaaagactttgtccataagacgaagggccaaattaatgacatatttcaaaaactcaacatatttgatcagtctttttatgatctatcgctgcaaaccagtgaaatcaaagaagaagaaaaggaactgagaagaactacatataaactacaagtcaaaaatgaagaggtaaagaatatgtcacttgaactcaactcaaaacttgaaagcctcctagaagaaaaaattctacttcaacaaaaagtgaaatatttagaagagcaactaactaacttaattcaaaatcaacctgaaactccagaacacccagaagtaacttcacttaaaacttttgtagaaaaacaagataatagcatcaaagaccttctccagaccgtggaagaccaatataaacaattaaaccaacagcatagtcaaataaaagaaatagaaaatcagctcagaaggactagtattcaagaacccacagaaatttctctatcttccaagccaagagcaccaagaactactccctttcttcagttgaatgaaataagaaatgtaaaacatgatggcattcctgctgaatgtaccaccatttataacagaggtgaacatacaagtggcatgtatgccatcagacccagcaactctcaagtttttcatgtctactgtgatgttatatcaggtagtccatggacattaattcaacatcgaatagatggatcacaaaacttcaatgaaacgtgggagaactacaaatatggttttgggaggcttgatggagaattttggttgggcctagagaagatatactccatagtgaagcaatctaattatgttttacgaattgagttggaagactggaaagacaacaaacattatattgaatattctttttacttgggaaatcacgaaaccaactatacgctacatctagttgcgattactggcaatgtccccaatgcaatcccggaaaacaaagatttggtgttttctacttgggatcacaaagcaaaaggacacttcaactgtccagagggttattcaggaggctggtggtggcatgatgagtgtggagaaaacaacctaaatggtaaatataacaaaccaagagcaaaatctaagccagagaggagaagaggattatcttggaagtctcaaaatggaaggttatactctataaaatcaaccaaaatgttgatccatccaacagattcagaaagctttgaatga

SEQ ID NO: 163 - protein, 455 aa, Mus musculus
MHTIKLFLFVVPLVIASRVDPDLSSFDSAPSEPKSRFAMLDDVKILANGLLQLGHGLKDFVHKTKGQINDIFQKLNIFDQSFYDLSLRTNEIKEEEKELRRTTSTLQVKNEEVKNMSVELNSKLESLLEEKTALQHKVRALEEQLTNLILSPAGAQEHPEVTSLKSFVEQQDNSIRELLQSVEEQYKQLSQQHMQIKEIEKQLRKTGIQEPSENSLSSKSRAPRTTPPLQLNETENTEQDDLPADCSAVYNRGEHTSGVYTIKPRNSQGFNVYCDTQSGSPWTLIQHRKDGSQDFNETWENYEKGFGRLDGEFWLGLEKIYAIVQQSNYILRLELQDWKDSKHYVEYSFHLGSHETNYTLHVAEIAGNIPGALPEHTDLMFSTWNHRAKGQLYCPESYSGGWWWNDICGENNLNGKYNKPRTKSRPERRRGIYWRPQSRKLYAIKSSKMMLQPTT

SEQ ID NO: 164 - protein, 406 aa, Homo sapiens
MSGAPTAGAALMLCAATAVLLSAQGGPVQSKSPRFASWDEMNVLAHGLLQLGQGLREHAERTRSQLSALERRLSACGSACQGTEGSTDLPLAPESRVDPEVLHSLQTQLKAQNSRIQQLFHKVAQQQRHLEKQHLRIQHLQSQFGLLDHKHLDHEVAKPARRKRLPEMAQPVDPAHNVSRLHRLPRDCQELFQVGERQSGLFEIQPQGSPPFLVNCKMTSDGGWTVIQRRHDGSVDFNRPWEAYKAGFGDPHGEFWLGLEKVHSITGDRNSRLAVQLRDWDGNAELLQFSVHLGGEDTAYSLQLTAPVAGQLGATTVPPSGLSVPFSTWDQDHDLRRDKNCAKSLSGGWWFGTCSHSNLNGQYFRSIPQQRQKLKKGIFWKTWRGRYYPLQATTMLIQPMAAEAAS

SEQ ID NO: 165 - protein, 386 aa, synthetic construct
SRIDQDNSSFDSLSPEPKSRFAMLDDVKILANGLLQLGHGLKDFVHKTKGQINDIFQKLNIFDQSFYDLSLQTSEIKEEEKELRRTTYKLQVKNEEVKNMSLELNSKLESLLEEKILLQQKVKYLEEQLTNLIQNQPETPEHPEVTSLKTFVEEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 166 - protein, 235 aa, synthetic construct
GAPSRVDPDLSSFDSAPSEPKSRFAMLDDVKILANGLLQLGHGLKDFVHKTKGQINDIFQKLNIFDQSFYDLSLRTNEIKEEEKELRRTTSTLQVKNEEVKNMSVELNSKLESLLEEKTALQHKVRALEEQLTNLILSPAGAQEHPEVTSLKSFVEQQDNSIRELLQSVEEQYKQLSQQHMQIKEIEKQLRKTGIQEPSENSLSSKSRAPRTTPPLQLNETENTEQDASHHHHHH

SEQ ID NO: 167 - protein, 182 aa, synthetic construct
SRIDQDNSSFDSVSPEPKSRFAMLDDVKILANGLLQLGHGLKDFVHKTKGQINDIFQKLNIFDQSFYDLSLQTSEIKEEEKELRRTTYKLQVKNEEVKNMSLELNSKLESLLEEKILLQQKVKYLEEQLTNLIQNQPATPEHPEVTSLKSFVEKEQKLISEEDLGGEQKLISEEDLHHHHHH

SEQ ID NO: 168 - protein, 33 aa, synthetic construct
RFASWDEMNVLAHGLLQLGQGLREHAERTRSQL

SEQ ID NO: 169 - protein, 33 aa, synthetic construct
RFAMLDDVKILANGLLQLGHGLKDFVHKTKGQI

SEQ ID NO: 170 - protein, 26 aa, synthetic construct
EPKSRFAMLDDVKILANGLLQLGHGL

SEQ ID NO: 171 - protein, 7 aa, synthetic construct
AGSSPGG

SEQ ID NO: 172 - protein, 5 aa, synthetic construct
GGGGS

SEQ ID NO: 173 - protein, 10 aa, synthetic construct
GPSSGAPPPK

SEQ ID NO: 174 - protein, 6 aa, synthetic construct
GGGGSK

SEQ ID NO: 175 - protein, 455 aa, Rattus norvegicus
MHTIKLLLFVVPLVISSRVDPDLSPFDSVPSEPKSRFAMLDDVKILANGLLQLGHGLKDFVHKTKGQINDIFQKLNIFDQCFYDLSLQTNEIKEEEKELRRTTSKLQVKNEEVKNMSLELNSKLESLLEEKMALQHRVRALEEQLTSLVQNPPGAREHPEVTSLKSFVEQQDNSIRELLQSVEEQYKQLSQQHIQIKEIENQLRKTGIQEPTENSLYSKPRAPRTTPPLHLKEAKNIEQDDLPADCSAIYNRGEHTSGVYTIRPSSSQVFNVYCDTQSGTPRTLIQHRKDGSQNFNQTWENYEKGFGRLDGEFWLGLEKIYAIVKQSNYILRLELQDWKDSKHYAEYSFHLGNHETNYTLHVAEIAANIPEALPEHRDLMFSTWDHRAKGQLYCPESYSGGWWFSDMCGENNLNGKYNKPRAKSKPERRRGISWRPRGGKLYSIKSSKMMLQPTT

SEQ ID NO: 176 - protein, 252 aa, synthetic construct
SRVDPDLSPFDSVPSEPKSRFAMLDDVKILANGLLQLGHGLKDFVHKTKGQINDIFQKLNIFDQCFYDLSLQTNEIKEEEKELRRTTSKLQVKNEEVKNMSLELNSKLESLLEEKMALQHRVRALEEQLTSLVQNPPGAREHPEVTSLKSFVEQQDNSIRELLQSVEEQYKQLSQQHIQIKEIENQLRKTGIQEPTENSLYSKPRAPRTTPPLHLKEAKNIEQDEQKLISEEDLGGEQKLISEEDLHHHHHH

SEQ ID NO: 177 - protein, 210 aa, Macaca fascicularis
MFTIKLLLFIVPLVISSRIDQDNSSFDSVSPEPKSRFAMLDDVKILANGLLQLGHGLKDFVHKTKGQINDIFQKLNIFDQSFYDLSLQTSEIKEEEKELRRTTYKLQVKNEEVKNMSLELNSKLESLLEEKILLQQKVKYLEEQLTNLIQNQPATPEHPEVTSLKSFVEKQDNSIKDLLQTVEEQYKQLNQQHSQIKEIENQLRMTNIQE

SEQ ID NO: 178 - protein, 356 aa, synthetic construct
GPVQSKSPRFASWDEMNVLAHGLLQLGQGLREHAERTRSQLSALERRLSACGSACQGTEGSTDLPLAPESRVDPEVLHSLQTQLKAQNSRIQQLFHKVAQQQRHLEKQHLRIQHLQSQFGLLDEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 179 - DNA, 363 bp, synthetic construct
  source 1..363: mol_type = other DNA
gaggtgcagcttttggagtctgggggaggcttggtacagcctggggggtccctgagactctcctgtgcagcctctggattcacctttagcacctatgccatgagctgggtccgccaggctccagggaaggggctggagggggtctcaggtattagtggtactggttatagaacatactacgcagactccgtgaagggccgattcaccatctccagagacaattccaagaactcgctgtatctgcaaatgaacagcctgagagccgaggacacggccgtatattactgtgcgaaagatcggggcttactatggttcggggaattaacctactggggccagggaaccctggtcaccgtctcctca

SEQ ID NO: 180 - protein, 121 aa, synthetic construct
EVQLLESGGGLVQPGGSLRLSCAASGFTFSTYAMSWVRQAPGKGLEGVSGISGTGYRTYYADSVKGRFTISRDNSKNSLYLQMNSLRAEDTAVYYCAKDRGLLWFGELTYWGQGTLVTVSS

SEQ ID NO: 181 - DNA, 24 bp, synthetic construct
  source 1..24: mol_type = other DNA
ggattcacctttagcacctatgcc

SEQ ID NO: 182 - protein, 8 aa, synthetic construct
GFTFSTYA

SEQ ID NO: 183 - DNA, 24 bp, synthetic construct
  source 1..24: mol_type = other DNA
attagtggtactggttatagaaca

SEQ ID NO: 184 - protein, 8 aa, synthetic construct
ISGTGYRT

SEQ ID NO: 185 - DNA, 42 bp, synthetic construct
  source 1..42: mol_type = other DNA
gcgaaagatcggggcttactatggttcggggaattaacctac

SEQ ID NO: 186 - protein, 14 aa, synthetic construct
AKDRGLLWFGELTY

SEQ ID NO: 187 - DNA, 318 bp, synthetic construct
  source 1..318: mol_type = other DNA
gacatccagatgacccagtctccttccaccctgtctgcatctgtaggagacagagtcaccatcacttgccgggccagtcagagtattaataactggttggcctggtatcaacagaaaccagggaaggcccctaacctcctgatctataaggcgtctagtttagaaagtggggtcccatcaaggttcagcggcagtggatctgggacagaattcactctcaccatcagcagcctgcagcctgatgattttgcaacttattactgccaacaatataatgattattggacgttcggccaagggaccaaggtggaaatcaaa

SEQ ID NO: 188 - protein, 106 aa, synthetic construct
DIQMTQSPSTLSASVGDRVTITCRASQSINNWLAWYQQKPGKAPNLLIYKASSLESGVPSRFSGSGSGTEFTLTISSLQPDDFATYYCQQYNDYWTFGQGTKVEIK

SEQ ID NO: 189 - DNA, 18 bp, synthetic construct
  source 1..18: mol_type = other DNA
cagagtattaataactgg

SEQ ID NO: 190 - protein, 6 aa, synthetic construct
QSINNW

SEQ ID NO: 191-192 - skipped (2 entries)

SEQ ID NO: 193 - DNA, 24 bp, synthetic construct
  source 1..24: mol_type = other DNA
caacaatataatgattattggacg

SEQ ID NO: 194 - protein, 8 aa, synthetic construct
QQYNDYWT

SEQ ID NO: 195 - protein, 453 aa, synthetic construct
EVQLVESGGGVIQPGGSLRLSCAASGFTFDDYAMNWVRQGPGKGLEWVSAISGDGGSTYYADSVKGRFTISRDNSKNSLYLQMNSLRAEDTAFFYCAKDLRNTIFGVVIPDAFDIWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 196 - protein, 214 aa, synthetic construct
DIQMTQSPSTLSASVGDRVTITCRASQSIRSWLAWYQQKPGKAPKLLIYKASSLESGVPSRFSGSGSGTEFTLTISSLQPDDFATYYCQQYNSYSYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2026/0139045 A1
(43) Pub. Date: May 21, 2026

(54) STABILIZED FORMULATIONS CONTAINING ANTI-ANGPTL3 ANTIBODIES

(71) Applicant: Regeneron Pharmaceuticals, Inc., Tarrytown, NY (US)

(72) Inventors: Dingjiang LIU, Pleasantville, NY (US); Andria SKINNER, Mohegan Lake, NY (US)

(21) Appl. No.: 19/455,271

(22) Filed: Jan. 21, 2026

Related U.S. Application Data
(63) Continuation of application No. 16/882,280, filed on May 22, 2020, now Pat. No. 12,559,551.
(60) Provisional application No. 62/852,643, filed on May 24, 2019.

(51) Int. Cl. C07K 16/22; A61K 9/00

(52) U.S. Cl. CPC C07K 16/22; A61K 9/0019; C07K 2317/565; C07K 2317/94

(57) ABSTRACT

The present invention provides stable pharmaceutical formulations comprising a human antibody that specifically binds to human angiopoietin-like protein 3 (ANGPTL3). The formulations may contain, in addition to an anti-ANGPTL3 antibody, a buffer; an organic cosolvent; at least one viscosity modifier, and optionally at least one amino acid. The pharmaceutical formulations of the present invention can be administered via intravenous infusion or subcutaneously and exhibit a substantial degree of antibody stability after storage for several months.

### CROSS-REFERENCE TO RELATED APPLICATIONS

This application is a continuation of U.S. application Ser. No. 16/882,280, filed May 22, 2020, which claims the benefit under 35 U.S.C. § 119(e) of U.S. provisional application No. 62/852,643 filed on May 24, 2019. The disclosure of the aforementioned applications is herein incorporated by referenced in its entirety. To the extent appropriate, a claim of priority is made to each of the above-disclosed applications.

### REFERENCE TO A SEQUENCE LISTING XML

This application includes a Sequence Listing in electronic format entitled “40848_0092USC1_SL.xml” which was created Jan. 2, 2026 and which has a size of 172,406 bytes. The contents of the XML file “40848_0092USC1_SL.xml” are incorporated by reference herein.

### FIELD OF THE INVENTION

The present invention relates to the field of therapeutic antibody formulations. More specifically, the present invention relates to the field of pharmaceutical formulations comprising a human antibody that specifically binds to human angiopoietin-like (protein) 3 (ANGPTL3).

### BACKGROUND OF THE INVENTION

Therapeutic macromolecules (e.g., antibodies) must be formulated in a manner that not only makes the molecules suitable for administration to patients, but also maintains their stability during storage and subsequent use. For example, therapeutic antibodies in liquid solution are prone to degradation, aggregation, or undesired chemical modifications, unless the solution is properly formulated. The stability of an antibody in liquid formulation depends not only on the kinds of excipients used in the formulation, but also on the amounts and proportions of the excipients relative to one another. Furthermore, other considerations aside from stability must be taken into account when preparing a liquid antibody formulation. Examples of such additional considerations include the viscosity of the solution and the concentration of antibody that can be accommodated by a given formulation, as well as the visual quality or appeal of the formulation. Thus, when formulating a therapeutic antibody, great care must be taken to arrive at a formulation that remains stable, contains an adequate concentration of antibody, and possesses a suitable viscosity, as well as other properties that enable the formulation to be conveniently administered to patients.

Antibodies to angiopoietin-like protein 3 (ANGPTL3) are one example of a therapeutically relevant macromolecule that requires proper formulation. Anti-ANGPTL3 antibodies are clinically useful for the treatment of diseases or disorders associated with lipid metabolism, cardiovascular diseases or disorders, and diseases or disorders associated with angiogenesis.

The amino acid and nucleotide sequences of human ANGPTL3 are shown in SEQ ID NOS: 161 and 162, respectively. Exemplary anti-ANGPTL3 antibodies are described, for example, in U.S. Pat. No. 9,018,356B2, WO2008/073300, and U.S. Pat. No. 7,935,796.

_Although anti-ANGPTL3 antibodies are known, there remains a need in the art for novel pharmaceutical formulations comprising such antibodies that are sufficiently stable and suitable for administration to patients.

### BRIEF SUMMARY OF THE INVENTION

For many commercialized monoclonal antibodies, the final product presentation is dictated by the administration method. One such method is based on patients' preference for self-administration and less frequent dosing. Self-administration of a subcutaneous injection is one preferred means of administering parenteral products developed for the long-term treatment of many diseases. Subcutaneous (SC) injections necessitate dosing in ≤2 mL total volume, preferably ≤1 mL total volume. Less frequent dosing requires a higher concentration of drug per dose and, consequently, a higher protein concentration formulation. Thus, to enable less frequent dosing, high concentrations of drug (>150 mg per dose) that can be delivered in 1 mL are desired. High concentration formulations also enable smaller dosing volumes. For example, to deliver 15 mg/kg of drug to a 100 kg patient, i.e. 1500 mg of drug, 150 mL of a 10 mg/mL formulation is needed, whereas only 10 mL of a 150 mg/mL formulation is needed. Thus, high concentration formulations are preferred for the small injection volumes they enable.

It is important to consider both the stability and the viscosity of this higher protein concentration formulation. Because the relationship between protein concentration and viscosity is exponential, small differences in protein concentration may have large effects on viscosity and impact the ability of the patient to deliver the drug. The steepness of the curve depicting viscosity (y-axis) in relation to protein concentration (x-axis) can be affected by the addition of excipients, especially ones that increase (e.g., sugars) or decrease (e.g., salts) the viscosity and temperature. Furthermore, the viscosity is directly related to the ability to deliver the drug through a syringe. The sustaining force is the required force to continuously dispense the contents of a pre-filled syringe. It is measured using a Syringe Force Tester (Instron). The relationship between sustaining force and viscosity is linear.

Self-administration with a pre-filled syringe or auto injector necessitates a formulation with low viscosity (typically less than about 20 cPoise). There is, thus, a need to identify viscosity-reducing excipients and evaluate their effect on the rheological properties and stability of antibody (specifically, anti-ANGPTL3 antibody) formulations. This data can be used to enable development of an amino acid-based high-concentration liquid formulation with acceptable viscosity that can be used in pre-filled syringes and device development.

The present invention satisfies the afore-mentioned need by providing stable pharmaceutical formulations comprising a fully human monoclonal antibody that specifically binds to human angiopoietin-like protein 3 (ANGPTL3). H4H1276S is a fully human monoclonal antibody that targets ANGPTL3, an important protein that inhibits lipoprotein lipase (LPL) when active. The inhibition of ANGPTL3 by H4H1276S restores LPL activity and promotes processing of triglycerides and VLDL. H4H1276S is, thus, potentially indicated for several disease pathways, including severe hypertriglyceridemia and homozygous familial hypercholesterolemia.

In one aspect, a stable high-concentration liquid pharmaceutical formulation of low viscosity is provided, comprising: (i) a human antibody that specifically binds to human angiopoietin-like protein 3 (ANGPTL3); (ii) a buffer; (iii) an organic cosolvent; and (iv) at least one viscosity modifier. In one embodiment, the stable high-concentration liquid pharmaceutical formulation further comprises at least one amino acid. In another embodiment, the formulation comprises a stabilizer. In another aspect, a stable high-concentration liquid pharmaceutical formulation of low viscosity is provided, comprising: (i) a human antibody that specifically binds to human angiopoietin-like protein 3 (ANGPTL3); (ii) a buffer; (iii) an organic cosolvent; and (iv) at least two viscosity modifiers. In one embodiment, the stable high-concentration liquid pharmaceutical formulation further comprises at least one amino acid. In another embodiment, the formulation comprises a stabilizer. The term “viscosity modifier” includes viscosity-reducing agents or excipients.

In various embodiments, the antibody is provided at a concentration from about 5±0.75 mg/mL to about 250±37.5 mg/mL. In one embodiment, the antibody is provided at a concentration of 12.5 mg/mL±1.85 mg/mL, or about 12.5 mg/mL. In another embodiment, the antibody is provided at a concentration of 25 mg/mL±3.75 mg/mL, or about 25 mg/mL. In another embodiment, the antibody is provided at a concentration of 50 mg/mL±7.5 mg/mL, or about 50 mg/mL. In another embodiment, the antibody is provided at a concentration of 100 mg/mL±15 mg/mL, or about 100 mg/mL. In one embodiment, the antibody is provided at a concentration of 150 mg/mL±22.5 mg/mL, or about 150 mg/mL. In another embodiment, the antibody is provided at a concentration of 165 mg/mL±24.75 mg/mL, or about 165 mg/mL. In another embodiment, the antibody is provided at a concentration of 175 mg/mL±26.25 mg/mL, or about 175 mg/mL. In another embodiment, the antibody is provided at a concentration of 200 mg/mL±30 mg/mL, or about 200 mg/mL.

In certain embodiments, the formulation comprises any one of the anti-ANGPTL3 antibodies disclosed in U.S. Pat. No. 9,018,356B2, incorporated herein in its entirety. In certain embodiments, the anti-ANGPTL3 antibody comprises (a) a heavy chain variable region (HCVR) comprising heavy chain complementarity determining regions 1, 2 and 3 (HCDR1-HCDR2-HCDR3) each comprising a sequence of SEQ ID NO: 68, SEQ ID NO: 70 and SEQ ID NO: 72, respectively; and (b) a light chain variable region (LCVR) comprising light chain complementarity determining regions 1, 2 and 3 (LCDR1-LCDR2-LCDR3) each comprising a sequence of SEQ ID NO: 76, KAS and SEQ ID NO: 80, respectively. In one embodiment, the antibody comprises a HCVR comprising the amino acid sequence of SEQ ID NO: 66 and a LCVR comprising the amino acid sequence of SEQ ID NO: 74. In another embodiment, the antibody comprises a HCVR having at least about 90% sequence identity to SEQ ID NO: 66 and a LCVR having at least about 90% sequence identity to SEQ ID NO: 74. In still another embodiment, the antibody comprises a HCVR having at least about 95% sequence identity to SEQ ID NO: 66 and a LCVR having at least about 95% sequence identity to SEQ ID NO: 74.

In one embodiment, the pH of the liquid formulation is pH 6.0±0.5, pH 6.0±0.4, pH 6.0±0.3, pH 6.0±0.2, pH 6.0±0.1, pH 6.0±0.05, pH 6.0±0.01, or pH 6.0. In one embodiment, the pH of the liquid formulation is about pH 6.0±0.3.

In one embodiment, the buffer is histidine. In certain embodiments, the histidine is at a concentration of from 5 mM±1 mM to 50 mM±10 mM, preferably from 5 mM±1 mM to 25 mM±5 mM. In one embodiment, the histidine is at a concentration of 10 mM±2 mM or 10 mM±1 mM or about 10 mM. In another embodiment, the histidine is at a concentration of 20 mM±4 mM or 20 mM±2 mM or about 20 mM. In still another embodiment, the histidine is at a concentration of 40 nM±8 mM or 40 nM±4 mM or about 40 nM.

In certain embodiments, the organic cosolvent is a nonionic polymer containing a polyoxyethylene moiety. In one embodiment, the organic solvent is a surfactant. In some embodiments, the organic cosolvent is any one or more of polysorbate, poloxamer 188 and polyethylene glycol 3350. In one embodiment, the organic cosolvent is polysorbate 80. In one embodiment, the organic cosolvent is polysorbate 20.

In one embodiment, the organic cosolvent is at a concentration of from about 0.01%±0.005% to about 1%±0.5% “weight to volume” or “w/v”, wherein, e.g., 0.1 g/ml=10% and 0.01 g/ml=1%. In certain embodiments, the organic solvent is polysorbate at a concentration of from 0.05%±0.025% to 0.5%±0.25% (w/v). In one embodiment, the organic cosolvent is polysorbate 80, which is at a concentration of 0.2%±0.1% w/v, or about 0.2%. In another embodiment, the organic cosolvent is polysorbate 80, which is at a concentration of 0.1%±0.05% w/v or about 0.1% w/v. In one embodiment, the organic cosolvent is polysorbate 20, which is at a concentration of 0.2%±0.1% w/v, or about 0.2%. In another embodiment, the organic cosolvent is polysorbate 20, which is at a concentration of 0.1%±0.05% w/v or about 0.1% w/v.

In certain embodiments, a stabilizer is included in the formulation. In one embodiment, the stabilizer is a sugar. In another embodiment, the sugar is sucrose. In various embodiments, the stabilizer is at a concentration of from 1%±0.2% w/v to 20%±4% w/v, from 5%±1% w/v to 15%±3% w/v, or from 1%±0.2% to 10%±2% w/v. In one embodiment, the stabilizer is sucrose at a concentration of 5%±1% w/v or about 5% w/v. In another embodiment, the stabilizer is sucrose at a concentration of 9%±1.8% w/v or about 9% w/v. In another embodiment, the stabilizer is sucrose at a concentration of 10%±2% w/v or about 10% w/v.

In one embodiment, at least one amino acid is included in the formulation. In one embodiment, the amino acid is L-proline. In certain embodiments, the amino acid is at a concentration of from 1%±0.2% to 5%±1% w/v. In one embodiment, the amino acid is proline at a concentration of 1.5%±0.3% or about 1.5%. In one embodiment, the amino acid is proline at a concentration of 3%±0.6%, or about 3%.

In one embodiment, the at least one viscosity modifier is an excipient selected from the group consisting of: Arginine-HCl, Sodium Chloride, Histidine-HCl, Sodium Acetate, Calcium Chloride, Magnesium Chloride, Calcium Acetate, and Magnesium Acetate. In one embodiment, the viscosity modifier is Arginine-HCl. In certain embodiments, the viscosity modifier is at a concentration of from 25 mM to about 75 mM. In one embodiment, the viscosity modifier is Arginine-HCl at a concentration of 50 mM to about 75 mM.

In certain embodiments, the viscosity of the liquid pharmaceutical formulation at 25° C. is less than or equal to about 20 cPoise±10%. In certain embodiments, the viscosity at 25° C. is between 1.0 cPoise±10% and 20 cPoise±10%. In certain embodiments, the viscosity of the liquid pharmaceutical formulation is ≤15 cPoise. In certain embodiments, the viscosity of the liquid pharmaceutical formulation is ≤20 cPoise. In certain embodiments, the viscosity of the liquid pharmaceutical formulation is ≤10 cPoise. In certain embodiments, the viscosity at 25° C. is 5 cPoise±10%, 6.0 cPoise±10%, 7.0 cPoise±10%, 7.1 cPoise±10%, 7.2 cPoise±10%, 7.9 cPoise±10%, 8.3 cPoise±10%, 9.0 cPoise±10%, 9.6 cPoise±10%, 10.0 cPoise±10%, 10.6 cPoise±10%, 11.4 cPoise±10%, 11.6 cPoise±10%, 11.8 cPoise±10%, 12.0 cPoise±10%, 13.0 cPoise±10%, 14.0 cPoise±10%, 15.0 cPoise±10%, or 16 cPoise±10%.

In one aspect, a stable liquid pharmaceutical formulation of low-viscosity is provided, comprising: (i) from 5±0.75 mg/ml to 250±37.5 mg/ml of a human antibody that specifically binds to human ANGPTL3; (ii) from 0 mM to 40±8 mM histidine; (iii) from 0% to 0.5%±0.25% (w/v) polysorbate 80; (iv) from 50±10 mM to 75±15 mM Arginine-HCl; and (v) from 0 to 5%±1% proline, at a pH of from about 5.3 to about 6.7; wherein the anti-ANGPTL3 antibody comprises a heavy chain variable region (HCVR) and a light chain variable region (LCVR) such that the HCVR/LCVR combination comprises heavy and light chain complementarity determining regions (HCDR1-HCDR2-HCDR3/LCDR1-LCDR2-LCDR3), which comprise the amino acid sequences of SEQ ID NOs: 68-70-72/SEQ ID NOs: 76-KAS-80, respectively. In one embodiment, the anti-ANGPTL3 antibody comprises a heavy chain variable region (HCVR) and light chain variable region (LCVR) comprising an amino acid sequence of SEQ ID NO: 66 and SEQ ID NO: 74, respectively. In certain embodiments, the anti-PD1 antibody comprises a Fc region elected from the group consisting of human IgG1, IgG2, IgG3, and IgG4 isotypes. In one embodiment, the antibody comprises a human IgG4 isotype.

In certain embodiments, a stable, low-viscosity liquid pharmaceutical formulation is provided, comprising: (i) from 5±0.75 mg/ml to 250±37.5 mg/ml of a human antibody that specifically binds to human ANGPTL3; (ii) from 0 mM to 40±8 mM histidine; (iii) from 0% to 0.5%±0.25% (w/v) polysorbate 80; (iv) from 50±10 mM to 75±15 mM Arginine-HCl; and (v) from 0 to 5%±1% proline, at a pH of from about 5.3 to about 6.7; wherein the anti-ANGPTL3 antibody comprises a HCVR and a LCVR, wherein the HCVR has at least about 90% sequence identity to SEQ ID NO: 66 and/or the LCVR has at least about 90% sequence identity to SEQ ID NO: 74.

In certain embodiments, a stable, low-viscosity liquid pharmaceutical formulation is provided, comprising: (i) from 5±0.75 mg/ml to 250±37.5 mg/ml of a human antibody that specifically binds to human ANGPTL3; (ii) from 0 mM to 40±8 mM histidine; (iii) from 0% to 0.5%±0.25% (w/v) polysorbate 80; (iv) from 50±10 mM to 75±15 mM Arginine-HCl; and (v) from 0 to 5%±1% proline, at a pH of from about 5.3 to about 6.7; wherein the anti-ANGPTL3 antibody comprises a HCVR and a LCVR, wherein the HCVR comprises an amino acid sequence of SEQ ID NO: 66 having no more than five amino acid substitutions, and wherein the LCVR comprises an amino acid sequence of SEQ ID NO: 74 having no more than two amino acid substitutions.

In certain embodiments, the formulations of any of the preceding aspects has an attribute selected from the group consisting of: (i) the formulation is stable to long-term storage at 25° C., 5° C., −20° C., −30° C. and −80° C., as described herein; (ii) the formulation is stable to agitation stress as described herein; (iii) the formulation is low-viscosity (viscosity less than about 20 cPoise, preferably less than about 15 cPoise); (iii) the formulation is stable even with up to ±50% variation in the formulation excipient concentrations, as described herein; (iv) the formulation is iso-osmolar to physiologic conditions; (iv) the formulation is stable to and compatible with subcutaneous delivery devices and procedures; and (v) the formulation is stable to long-term storage in a prefilled syringe.

In certain embodiments of this aspect, a stable liquid formulation is provided, comprising: (i) from 5±0.75 mg/ml to 250±37.5 mg/ml of a human antibody that specifically binds to human ANGPTL3; (ii) from 5 mM±1 mM to 20±4 mM histidine; (iii) from 0.05%±0.025% to 0.3%±0.15% (w/v) polysorbate 80; (iv) from 50±5 mM to 75±7.5 mM Arginine-HCl; and (v) from 1%±0.2% to 5%±1% proline, at a pH of about 6.0, wherein the antibody comprises a HCVR/LCVR comprising an amino acid sequence pair of SEQ ID NOs: 66/74. In one embodiment, the stable liquid formulation of this aspect has a viscosity less than about 20 cP. In another embodiment, the stable liquid formulation of this aspect has a viscosity less than about 15 cP.

In one embodiment of this aspect, the stable liquid formulation comprises (i) 50±7.5 mg/ml of an anti-ANGPTL3 antibody; (ii) 10±2 mM histidine; (iii) 0.1%±0.05% (w/v) polysorbate 80; (iv) 3%±0.6% proline; and (v) 70±5 mM Arginine-HCl, at a pH of 6.0±0.3, wherein the antibody comprises a HCVR/LCVR comprising an amino acid sequence pair of SEQ ID NOs: 66/74.

In another embodiment, the stable liquid formulation comprises (i) 100±15 mg/mL of an anti-ANGPTL3 antibody; (ii) 10±2 mM histidine; (iii) 0.1%±0.05% (w/v) (w/v) polysorbate 80; (iv) 3%±0.6% proline; and (v) 70±5 mM Arginine-HCl, at a pH of 6.0±0.3, wherein the antibody comprises a HCVR/LCVR comprising an amino acid sequence pair of SEQ ID NOs: 66/74.

In another embodiment, the stable liquid formulation comprises (i) 150±22.5 mg/ml of an anti-ANGPTL3 antibody; (ii) 10±2 mM histidine; (iii) 0.1%±0.05% (w/v) polysorbate 80; (iv) 70±5 mM Arginine-HCl; and (v) 3%±0.6% proline, at a pH of 6.0±0.3, wherein the antibody comprises a HCVR/LCVR comprising an amino acid sequence pair of SEQ ID NOs: 66/74. In one embodiment of the formulations disclosed herein, the viscosity is less than about 20 cPoise, in another embodiment, less than about 15 cPoise.

In another embodiment of this aspect, the stable liquid formulation comprises (i) 175±26.25 mg/mL of an anti-ANGPTL3 antibody; (ii) 10±2 mM histidine; (iii) 0.1%±0.05% (w/v) polysorbate 80; (iv) 70±5 mM Arginine-HCl; and (v) 3%±0.6% proline, at a pH of 6.0±0.3, wherein the antibody comprises a HCVR/LCVR comprising an amino acid sequence pair of SEQ ID NOs: 66/74.

In another embodiment of this aspect, the stable liquid formulation comprises (i) 200±30.00 mg/mL of an anti-ANGPTL3 antibody; (ii) 10±2 mM histidine; (iii) 0.1%±0.05% (w/v) polysorbate 80; (iv) 70±5 mM Arginine-HCl; and (v) 3%±0.6% proline, at a pH of 6.0±0.3, wherein the antibody comprises a HCVR/LCVR comprising an amino acid sequence pair of SEQ ID NOs: 66/74.

In one embodiment of the formulations disclosed herein, the formulation additionally comprises 5%±1% (w/v) sucrose.

In one embodiment, after storage of the formulation at 45° for 21 days, ≥about 95% of the antibody is native, and ≥about 45% of the antibody is of the main charge form. In one embodiment, after storage of the formulation at 5° for 36 months, >about 98% of the antibody is native, and >about 55% of the antibody is of the main charge form. In one embodiment, after storage of the formulation at −20° for 9 months, >about 98% of the antibody is native, and >about 61% of the antibody is of the main charge form. In one embodiment, after storage of the formulation at −30° for 36 months, >about 98% of the antibody is native, and >about 56% of the antibody is of the main charge form.

The cation exchange chromatography elution profile of a monoclonal antibody generally includes three peaks: the early- and late-eluting peaks (the so-called acidic and basic variants, respectively), and the most abundant peak (in the middle) is called the main peak (or main charge form or variant).

In one aspect, a liquid pharmaceutical formulation of any of the preceding aspects is provided in a container. In one embodiment, the container is a polycarbonate vial. In another embodiment, the container is a glass vial. In one embodiment, the glass vial is a type 1 borosilicate glass vial with a fluorocarbon-coated butyl rubber stopper. In another embodiment, the container is a microinfuser. In another embodiment, the container is a syringe. In another embodiment, the container is a prefilled syringe. In one embodiment, the syringe comprises a fluorocarbon-coated plunger. In certain embodiments, the syringe is a 1 mL or 2.25 mL long glass syringe containing less than about 500 parts per billion of tungsten equipped with a 27-G needle, a fluorocarbon-coated butyl rubber stopper, and a latex-free, non-cytotoxic rubber tip cap. In one specific embodiment, the syringe is a 1 mL long glass syringe equipped with a 27-G thin wall needle, a FLUROTEC-coated 4023/50 rubber stopper, and a FM 27 rubber tip cap. In another specific embodiment, the syringe is a 1 mL or 3 mL plastic syringe fitted with a 27-G needle. In one embodiment, the plastic syringe is distributed by BECTON DICKINSON. In another embodiment, the container is a Type 1 clear glass with FluroTecr® coated 4432/50 chlorobutyl stopper.

In one aspect, a kit comprising a pharmaceutical composition of any one of the preceding aspects, a container, and instructions for use is provided. In one embodiment, the container is a prefilled syringe. In one embodiment, the syringe is a NUOVA OMPI 1 mL or 2.25 mL long glass syringe equipped with a 27-G thin wall needle, a FLUROTEC-coated 4023/50 rubber stopper, and a FM 27 rubber tip cap.

In certain embodiments, the present invention provides a prefilled syringe comprising a stable liquid pharmaceutical formulation comprising: (i) from 5±0.75 mg/ml to 250±37.5 mg/ml of a human antibody that specifically binds to human ANGPTL3; (ii) from 5 mM±1 mM to 20±4 mM histidine; (iii) from 0.05%±0.025% to 0.3%±0.15% (w/v) polysorbate 80; (iv) from 50±10 mM to 75±15 mM Arginine-HCl; and (v) from 1%±0.2% to 5%±1% proline, at a pH of 6.0±0.3, wherein the antibody comprises a HCVR/LCVR comprising an amino acid sequence pair of SEQ ID NOs: 66/74; wherein the formulation has an attribute selected from the group consisting of: (i) ≥about 98% of the antibody is in native form after storage at 5° C. for 36 months; (ii) ≥about 55% of the antibody is the main charge variant after storage at 5° C. for 36 months; (iii) the formulation is stable to agitation stress wherein ≥98% of the antibody is in native form after 120 minutes of agitation stress in a clear glass vial.

In certain embodiments the present invention provides a stable liquid pharmaceutical formulation comprising: (i) from 5±0.75 mg/ml to 250±37.5 mg/ml of a human antibody that specifically binds to human ANGPTL3; (ii) from 5 mM±1 mM to 20±4 mM histidine; (iii) from 0.05%±0.025% to 0.3%±0.15% (w/v) polysorbate 80; (iv) from 50±10 mM to 75±15 mM Arginine-HCl; and (v) from 1%±0.2% to 5%±1% proline, at a pH of 6.0±0.3, wherein the antibody comprises a HCVR/LCVR comprising an amino acid sequence pair of SEQ ID NOs: 66/74; wherein the formulation has an attribute selected from the group consisting of: (i) the formulation is stable to and compatible for use in subcutaneous and/or intravenous delivery devices; (ii) the formulation is chemically and physically stable to dilution with standard diluents known in the art (e.g., 0.9% sodium chloride or 5% dextrose); (iii) the formulation is stable to prefilled syringe or auto-injector format; and (iv) the formulation is compatible with standard infusion pumps (e.g., peristaltic pump, fluid displacement pump).

Other embodiments will become apparent from a review of the ensuing detailed description.

### BRIEF DESCRIPTION OF FIGURES

FIG. 1 graphically depicts the effect of H4H1276S concentration on viscosity.

FIG. 2 shows a table summarizing the effect of pH on the stability of 150 mg/mL H4H1276S incubated at 45° for 28 days. ^{a.} A sample passes color and visual appearance if it is clear to slightly opalescent, essentially free from visible particulates, colorless to pale yellow. ^{b.} Reported as a change in purity relative to the starting material. The starting material (no incubation) contains ≥98.4% native peak by SE-UPLC and ≥62.7% main peak by CEX-UPLC in all 5 formulations. CEX-UPLC, cation exchange ultra performance liquid chromatography; FDG, Formulation Development Group; HMW, high molecular weight; LMW, low molecular weight; OD, optical density; RP-UPLC, reversed phase ultra performance liquid chromatography; SE-UPLC, size exclusion ultra performance liquid chromatography.

FIG. 3 shows a table summarizing the effect of polysorbate 80 concentration on the stability of 150 mg/mL H4H1276S following agitation (120 minutes of vortexing). ^{a.} Sample passes color and visual appearance if it is clear to slightly opalescent, essentially free from visible particulates, colorless to pale yellow. ^{b.} Reported as a change in purity relative to the starting material. The starting material (no incubation) contains ≥98.4% native peak by SE-UPLC and ≥62.7% main peak by CEX-UPLC in all 12 formulations. CEX-UPLC, cation exchange ultra performance liquid chromatography; FDG, Formulation Development Group; HMW, high molecular weight; LMW, low molecular weight; OD, optical density; RP-UPLC, reversed phase ultra performance liquid chromatography; SE-UPLC, size exclusion ultra performance liquid chromatography.

FIG. 4 shows a table summarizing the effect of polysorbate 80 concentration on the stability of 150 mg/mL H4H1276S following incubation at 45° for 28 days. ^{a.} Sample passes color and visual appearance if it is clear to slightly opalescent, essentially free from visible particulates, colorless to pale yellow. ^{b.} Reported as a change in purity relative to the starting material. The starting material (no incubation) contains ≥98.4% native peak by SE-UPLC and ≥62.7% main peak by CEX-UPLC in all 5 formulations. CEX-UPLC, cation exchange ultra performance liquid chromatography; FDG, Formulation Development Group; HMW, high molecular weight; LMW, low molecular weight; OD, optical density; RP-UPLC, reversed phase ultra performance liquid chromatography; SE-UPLC, size exclusion ultra performance liquid chromatography.

FIG. 5 shows a table summarizing the effect of polysorbate 80 concentration on subvisible particle formation for 150 mg/mL H4H1276S following agitation (120 minutes of vortexing) or incubation at 45° for 28 days. ^{a.} Data were filtered using an ECD (μm) ≥5.00, Aspect Ratio<0.85 and Ignore Edge Particle filter. CEX-UPLC, cation exchange ultra performance liquid chromatography; FDG, Formulation Development Group; HMW, high molecular weight; LMW, low molecular weight; OD, optical density; RP-UPLC, reversed phase ultra performance liquid chromatography; SE-UPLC, size exclusion ultra performance liquid chromatography.

FIG. 6 shows a table summarizing the effect of sucrose and proline on the stability of H4H1276S following incubation at −20° C. for nine months. ^{a.} Corresponds to 175 mg/mL H4H1276S, 10 mM histidine, pH 6.0, 70 mM arginine-HCl formulation. ^{b.} Sample passes color and visual appearance if it is clear to slightly opalescent, essentially free from visible particulates, colorless to pale yellow. ^{c.} Reported as a change in purity relative to the starting material. The starting material (no incubation) contains ≥98.4% native peak by SE-UPLC and ≥62.7% main peak by CEX-UPLC in all 5 formulations. CEX-UPLC, cation exchange ultra performance liquid chromatography; FDG, Formulation Development Group; HMW, high molecular weight; LMW, low molecular weight; OD, optical density; RP-UPLC, reversed phase ultra performance liquid chromatography; SE-UPLC, size exclusion ultra performance liquid chromatography.

FIG. 7 shows a table summarizing the effect of sucrose and proline on the stability of H4H1276S following eight freeze/thaw cycles. ^{a.} Corresponds to 175 mg/mL H4H1276S, 10 mM histidine, pH 6.0, 70 mM arginine-HCl formulation. ^{b.} Sample passes color and visual appearance if it is clear to slightly opalescent, essentially free from visible particulates, colorless to pale yellow. ^{c.}Reported as a change in purity relative to the starting material. The starting material (no incubation) contains ≥98.4% native peak by SE-UPLC and ≥62.7% main peak by CEX-UPLC in all 5 formulations. CEX-UPLC, cation exchange ultra performance liquid chromatography; FDG, Formulation Development Group; HMW, high molecular weight; LMW, low molecular weight; OD, optical density; RP-UPLC, reversed phase ultra performance liquid chromatography; SE-UPLC, size exclusion ultra performance liquid chromatography.

FIG. 8 shows a table summarizing the effect of sucrose and proline on the stability of H4H1276S following incubation at 45° for 21 days. ^{a.} Corresponds to 175 mg/mL H4H1276S, 10 mM histidine, pH 6.0, 70 mM arginine-HCl formulation. ^{b.} Sample passes color and visual appearance if it is clear to slightly opalescent, essentially free from visible particulates, colorless to pale yellow. ^{c.} Reported as a change in purity relative to the starting material. The starting material (no incubation) contains ≥98.4% native peak by SE-UPLC and ≥62.7% main peak by CEX-UPLC in all 5 formulations. CEX-UPLC, cation exchange ultra performance liquid chromatography; FDG, Formulation Development Group; HMW, high molecular weight; LMW, low molecular weight; OD, optical density; RP-UPLC, reversed phase ultra performance liquid chromatography; SE-UPLC, size exclusion ultra performance liquid chromatography.

FIG. 9 shows a table summarizing the effect of sucrose and proline on the stability of H4H1276S following incubation at −30° C. for 36 months. ^{a.} Sample passes color and visual appearance if it is clear to slightly opalescent, essentially free from visible particulates, colorless to pale yellow. ^{b.} Reported as a change in purity relative to the starting material. The starting material (no incubation) contains 98.8% native peak by SE-UPLC for both formulations and 59.2% main peak for the sucrose formulation and 60.0% main peak for the proline formulation, as determined by CEX-UPLC. CEX-UPLC, cation exchange ultra performance liquid chromatography; FDG, Formulation Development Group; HMW, high molecular weight; LMW, low molecular weight; OD, optical density; RP-UPLC, reversed phase ultra performance liquid chromatography; SE-UPLC, size exclusion ultra performance liquid chromatography.

FIG. 10 shows a table summarizing the effect of sucrose and proline on the stability of H4H1276S following incubation at 5° C. for 36 months. ^{a.} Sample passes color and visual appearance if it is clear to slightly opalescent, essentially free from visible particulates, colorless to pale yellow. ^{b.} Reported as a change in purity relative to the starting material. The starting material (no incubation) contains 98.8% native peak by SE-UPLC for both formulations and 59.2% main peak for the sucrose formulation and 60.0% main peak for the proline formulation, as determined by CEX-UPLC. ^{c.} The average of three independent samples is reported. CEX-UPLC, cation exchange ultra performance liquid chromatography; FDG, Formulation Development Group; HMW, high molecular weight; LMW, low molecular weight; OD, optical density; RP-UPLC, reversed phase ultra performance liquid chromatography; SE-UPLC, size exclusion ultra performance liquid chromatography.

FIG. 11 graphically depicts the viscosity vs. H4H1276S concentration for the lead formulations.

FIGS. 12A and 12B graphically depict the relationship between H4H1276S concentration, temperature, and viscosity. The star and circle points at 150 mg/mL H4H1276S also correspond to the recommended storage temperature of 5° C. (circle) or recommended administration temperature of 25° C. (star). The star and circle points at 165 mg/mL H4H1276S correspond to a formulation at a +10% manufacturing specification. The formulation of FIG. 12A is 10 mM histidine, 70 mM arginine-HCl, 5% sucrose, and 0.1% polysorbate 80, pH 6. The formulation of FIG. 12B is 10 mM histidine, 70 mM arginine-HCl, 3% proline, and 0.1% polysorbate 80, pH 6.

FIGS. 13A and 13B are bar graphs showing the results of screening for viscosity-reducing excipients. In FIG. 13A, viscosity is measured for various excipients added to a base formulation, and the pH was adjusted for a few of the options. In FIG. 13B, viscosity is measured for various excipients added to a different base formulation.

FIG. 14 shows a bar graph, in which the relative increase in HMW species (quantified as a percent) is provided for various excipients, following 21 days incubation at 45° C.

FIGS. 15A and 15B depict the stability of H4H1276S formulations containing viscosity-reducing excipients in bar graph (FIG. 15A) and line graph (FIG. 15B) forms. In FIG. 15A, the relative increases in HMW and acidic species and viscosity are measured for formulations containing 70 mM Arg-HCl vs 25 mM Mg(OAc)₂ for various concentrations of sucrose and/or L-proline. In FIG. 15B, the frozen storage stability (−20° C.) is measured in terms of % HMW species over time.

FIGS. 16A and 16B are contour plots showing the temperature, H4H1276S concentration, and viscosity, as they relate to one another, for a formulation containing 10 mM histidine, pH 6, 70 mM Arg-HCl, and 3% (w/v) proline (FIG. 16A); and for a formulation containing 10 mM histidine, pH 6, 70 mM Arg-HCl, and 5% (w/v) sucrose (FIG. 16B).

FIG. 17 graphically depicts viscosity vs. H4H1276S concentration at 20° C. Viscosity was plotted as a function of protein concentration. The data was fit to an exponential curve using GraphPad Prism. The equation(s) can be used to predict viscosity based on known concentration, which is useful for defining manufacturing specifications

### DETAILED DESCRIPTION

Before the present formulations and methods are described, it is to be understood that this invention is not limited to particular formulations and methods, and experimental conditions described, as such formulations and methods and conditions may vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs. As used herein, the term “about”, when used in reference to a particular recited numerical value or range of values, means that the value may vary from the recited value by no more than 1%. For example, as used herein, the expression “about 100” includes 99 and 101 and all values in between (e.g., 99.1, 99.2, 99.3, 99.4, etc.). Although any methods and materials similar or equivalent to those described herein can be used in the practice or testing of the present invention, preferred methods and materials are now described. All publications mentioned herein are incorporated herein by reference in their entirety.

DEFINITIONS

| SEQ ID NO. from U.S. | |
|---|---|
| Application No. 16/882,280 | |
| ST25 Sequence Listing | Sequence |
| SEQ ID NO: 13 | gctgcatcc |
| SEQ ID NO: 14 | Ala Ala Ser |
| SEQ ID NO: 29 | aaggcgtct |
| SEQ ID NO: 30 | Lys Ala Ser |
| SEQ ID NO: 45 | gctgcatcc |
| SEQ ID NO: 46 | Ala Ala Ser |
| SEQ ID NO: 61 | aaggcgtct |
| SEQ ID NO: 62 | Lys Ala Ser |
| SEQ ID NO: 77 | aaggcgtct |
| SEQ ID NO: 93 | actacttcc |
| SEQ ID NO: 94 | Thr Thr Ser |
| SEQ ID NO: 109 | cctgcatcc |
| SEQ ID NO: 110 | Pro Ala Ser |
| SEQ ID NO: 125 | actgcatcc |
| SEQ ID NO: 126 | Thr Ala Ser |
| SEQ ID NO: 141 | aaggtttct |
| SEQ ID NO: 142 | Lys Val Ser |
| SEQ ID NO: 157 | gttgcagcc |
| SEQ ID NO: 158 | Val Ala Ala |
| SEQ ID NO: 191 | aaggcgtct |
| SEQ ID NO: 192 | Lys Ala Ser |

As used herein, the expression “pharmaceutical formulation” means a combination of at least one active ingredient (e.g., a small molecule, macromolecule, compound, etc. which is capable of exerting a biological effect in a human or non-human animal), and at least one inactive ingredient, which, when combined with the active ingredient or one or more additional inactive ingredients, is suitable for therapeutic administration to a human or non-human animal. The term “formulation”, as used herein, means “pharmaceutical formulation”, unless specifically indicated otherwise. The present invention provides pharmaceutical formulations comprising at least one therapeutic polypeptide. According to certain embodiments of the present invention, the therapeutic polypeptide is an antibody, or an antigen-binding fragment thereof, which binds specifically to human angiopoietin-like protein 3 (ANGPTL3).

The term “human angiopoietin-like protein 3” or “hANGPTL3”, as used herein, refers to ANGPTL3 having the nucleic acid sequence shown in SEQ ID NO: 162 and the amino acid sequence of SEQ ID NO: 161, or a biologically active fragment thereof.

The term “antibody”, as used herein, is intended to refer to immunoglobulin molecules comprised of four polypeptide chains, two heavy (H) chains and two light (L) chains inter-connected by disulfide bonds. Each heavy chain is comprised of a heavy chain variable region (HCVR) and a heavy chain constant region (C_{H}; comprised of domains C_{H}1, C_{H}2 and C_{H}3). Each light chain is comprised of a light chain variable region (LCVR) and a light chain constant region (C_{L}). The HCVR and LCVR can be further subdivided into regions of hypervariability, termed complementarity determining regions (CDR), interspersed with regions that are more conserved, termed framework regions (FR). Each HCVR and LCVR is composed of three CDRs and four FRs, arranged from amino-terminus to carboxy-terminus in the following order: FR1, CDR1, FR2, CDR2, FR3, CDR3, and FR4.

Substitution of one or more CDR residues or omission of one or more CDRs is also possible. Antibodies have been described in the scientific literature in which one or two CDRs can be dispensed with for binding. Padlan, et al. (1995 FASEB J. 9:133-139) analyzed the contact regions between antibodies and their antigens, based on published crystal structures, and concluded that only about one fifth to one third of CDR residues actually contact the antigen. Padlan also found many antibodies in which one or two CDRs had no amino acids in contact with an antigen (see also, Vajdos, et al. 2002 J Mol Biol 320:415-428).

CDR residues not contacting antigen can be identified from regions of Kabat CDRs lying outside Chothia CDRs, by molecular modeling and/or empirically. If a CDR or residue(s) thereof is omitted, it is usually substituted with an amino acid occupying the corresponding position in another human antibody sequence or a consensus of such sequences. Positions for substitution within CDRs and amino acids to substitute can also be selected empirically. Empirical substitutions can be conservative or non-conservative substitutions.

The term “human antibody”, as used herein, is intended to include antibodies having variable and constant regions derived from human germline immunoglobulin sequences. The human mAbs of the invention may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include mAbs in which CDR sequences derived from the germline of another mammalian species (e.g., mouse), have been grafted onto human FR sequences.

The fully-human anti-hANGPTL3 antibodies disclosed herein may comprise one or more amino acid substitutions, insertions and/or deletions in the framework and/or CDR regions of the heavy and light chain variable domains as compared to the corresponding germline sequences. Such mutations can be readily ascertained by comparing the amino acid sequences disclosed herein to germline sequences available from, for example, public antibody sequence databases. The antibodies and antigen-binding fragments thereof described herein are derived from any of the amino acid sequences disclosed herein, wherein one or more amino acids within one or more framework and/or CDR regions are mutated to the corresponding residue(s) of the germline sequence from which the antibody was derived, or to the corresponding residue(s) of another human germline sequence, or to a conservative amino acid substitution of the corresponding germline residues(s) (such sequence changes are referred to herein collectively as “germline mutations”).

A person of ordinary skill in the art, starting with the heavy and light chain variable region sequences disclosed herein, can easily produce numerous antibodies and antigen-binding fragments that comprise one or more individual germline back-mutations or combinations thereof. In certain embodiments, all of the framework and/or CDR residues within the V_{H} and/or V_{L} domains are mutated back to the residues found in the original germline sequence from which the antibody was derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2 or CDR3. In other embodiments, one or more of the framework and/or CDR residue(s) are mutated to the corresponding residue(s) of a different germline sequence (i.e., a germline sequence that is different from the germline sequence from which the antibody was originally derived).

Furthermore, the antibodies described herein may contain any combination of two or more germline mutations within the framework and/or CDR regions, e.g., wherein certain individual residues are mutated to the corresponding residues of a particular germline sequence while certain other residues that differ from the original germline sequence are maintained or are mutated to the corresponding residue of a different germline sequence. Once obtained, antibodies and antigen-binding fragments that contain one or more germline mutations can be easily tested for one or more desired property such as, improved binding specificity, increased binding affinity, improved or enhanced antagonistic or agonistic biological properties (as the case may be), reduced immunogenicity, etc. Antibodies and antigen-binding fragments obtained in this general manner are encompassed within the present invention.

The present invention also includes anti-ANGPTL3 antibodies comprising variants of any of the HCVR, LCVR, and/or CDR amino acid sequences disclosed herein having one or more conservative substitutions. For example, the present invention includes anti-ANGPTL3 antibodies having HCVR, LCVR, and/or CDR amino acid sequences with, e.g., 10 or fewer, 8 or fewer, 6 or fewer, 4 or fewer, 2 or 1, conservative amino acid substitution(s) relative to any of the HCVR, LCVR, and/or CDR amino acid sequences disclosed herein. In one embodiment, a HCVR comprises the amino acid sequence of SEQ ID NO:66 with 10 or fewer conservative amino acid substitutions therein. In another embodiment, a HCVR comprises the amino acid sequence of SEQ ID NO: 66 with 8 or fewer conservative amino acid substitutions therein. In another embodiment, a HCVR comprises the amino acid sequence of SEQ ID NO:66 with 6 or fewer conservative amino acid substitutions therein. In another embodiment, a HCVR comprises the amino acid sequence of SEQ ID NO:66 with 4 or fewer conservative amino acid substitutions therein. In yet another embodiment, a HCVR comprises the amino acid sequence of SEQ ID NO:66 with 2 or 1 conservative amino acid substitution(s) therein. In one embodiment, a LCVR comprises the amino acid sequence of SEQ ID NO: 74 with 10 or fewer conservative amino acid substitutions therein. In another embodiment, a LCVR comprises the amino acid sequence of SEQ ID NO:74 with 8 or fewer conservative amino acid substitutions therein. In another embodiment, a LCVR comprises the amino acid sequence of SEQ ID NO:74 with 6 or fewer conservative amino acid substitutions therein. In another embodiment, a LCVR comprises the amino acid sequence of SEQ ID NO:74 with 4 or fewer conservative amino acid substitutions therein. In yet another embodiment, a LCVR comprises the amino acid sequence of SEQ ID NO:74 with 2 or 1 conservative amino acid substitution(s) therein.

Unless specifically indicated otherwise, the term “antibody,” as used herein, shall be understood to encompass antibody molecules comprising two immunoglobulin heavy chains and two immunoglobulin light chains (i.e., “full antibody molecules”) as well as antigen-binding fragments thereof. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and (optionally) constant domains. Such DNA is known and/or is readily available from, e.g., commercial sources, DNA libraries (including, e.g., phage-display antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody will typically comprise at least one variable domain. The variable domain may be of any size or amino acid composition and will generally comprise at least one CDR that is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present invention include: (i) V_{H}-C_{H}1; (ii) V_{H}-C_{H}2; (iii) V_{H}-C_{H}3; (iv) V_{H}-C_{H}1-C_{H}2; (v) V_{H}-C_{H}1-C_{H}2-C_{H}3; (vi) V_{H}-C_{H}2-C_{H}3; (vii) V_{H}-C_{L}; (viii) V_{L}-C_{H}1; (ix) V_{L}-C_{H}2; (x) V_{L}-C_{H}3; (xi) V_{L}-C_{H}1-C_{H}2; (xii) V_{L}-C_{H}1-C_{H}2-C_{H}3; (xiii) V_{L}-C_{H}2-C_{H}3; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule. Moreover, an antigen-binding fragment of an antibody of the present invention may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

As with full antibody molecules, antigen-binding fragments may be monospecific or multispecific (e.g., bispecific). A multispecific antigen-binding fragment of an antibody will typically comprise at least two different variable domains, wherein each variable domain is capable of specifically binding to a separate antigen or to a different epitope on the same antigen. Any multispecific antibody format, including the exemplary bispecific antibody formats disclosed herein, may be adapted for use in the context of an antigen-binding fragment of an antibody of the present invention using routine techniques available in the art.

In certain embodiments, antibody or antibody fragments described herein may be conjugated to a therapeutic moiety (“immunoconjugate”), such as a cytotoxin, a chemotherapeutic drug, an immunosuppressant or a radioisotope.

The term “specifically binds,” or the like, means that an antibody or antigen-binding fragment thereof forms a complex with an antigen that is relatively stable under physiological conditions. Specific binding can be characterized by an equilibrium dissociation constant (K_{D}) of about 1×10⁻⁶ M or less (i.e., a smaller K_{D} denotes a tighter binding). Methods for determining whether two molecules specifically bind are well known in the art and include, for example, equilibrium dialysis, surface plasmon resonance, and the like. An isolated antibody that specifically binds hANGPTL3 may, however, exhibit cross-reactivity to other antigens, such as ANGPTL3 molecules from other species, for example, cynomolgus monkey ANGPTL3, mouse ANGPTL3, rat ANGPTL3, and/or hANGPTL4. Moreover, multi-specific antibodies (e.g., bispecifics) that bind to hANGPTL3 and one or more additional antigens are nonetheless considered antibodies that “specifically bind” hANGPTL3, as used herein.

The term “K_{D}”, as used herein, is intended to refer to the equilibrium dissociation constant of a particular antibody-antigen interaction.

An “isolated antibody”, as used herein, is intended to refer to an antibody that is substantially free of other mAbs having different antigenic specificities (e.g., an isolated antibody that specifically binds hANGPTL3 is substantially free of mAbs that specifically bind antigens other than hANGPTL3). An isolated antibody that specifically binds hANGPTL3 may, however, have cross-reactivity to other antigens, such as ANGPTL3 molecules from other species, such as cynomolgus monkey, mouse, rat, and/or other related proteins, such as human ANGPTL4.

A “neutralizing”, “blocking” or “abrogating” antibody, as used herein (or an antibody that “neutralizes”, “blocks” or “abrogates” ANGPTL3 activity), is intended to refer to an antibody whose binding to ANGPTL3 results in direct inhibition of at least one biological activity of ANGPTL3, as assessed by standard in vitro assays known in the art. The terms, “neutralize”, “inhibit”, “block” and “abrogate”, may be used herein interchangeably. A “non-blocking” antibody refers to an antibody whose binding to ANGPTL3 does not directly block a targeted activity of ANGPTL3 as assessed by standard in vitro assays, but yet may be an “interfering” antibody whose binding to ANGPTL3 results in indirect inhibition, reduction, attenuation, or other interference, of at least one biological activity of ANGPTL3 in vivo, e.g., by enhancing the clearance of ANGPTL3 from the circulation. Clearance of ANGPTL3 from the circulation can be particularly enhanced by a combination of at least two non-blocking antibodies. The neutralization, inhibition, abrogation, reduction, attenuation or interference, of a biological activity of ANGPTL3 can be assessed by measuring one or more indicators of ANGPTL3 biological activity by one or more of several standard in vitro or in vivo assays known in the art.

The term “surface plasmon resonance”, as used herein, refers to an optical phenomenon that allows for the analysis of real-time biospecific interactions by detection of alterations in protein concentrations within a biosensor matrix, for example using the BIACORE™ system (Pharmacia Biosensor AB, Uppsala, Sweden and Piscataway, N.J.).

The term “epitope” is a region of an antigen that is bound by an antibody. Epitopes may be defined as structural or functional. Functional epitopes are generally a subset of the structural epitopes and have those residues that directly contribute to the affinity of the interaction. Epitopes may also be conformational, that is, composed of non-linear amino acids. In certain embodiments, epitopes may include determinants that are chemically active surface groupings of molecules such as amino acids, sugar side chains, phosphoryl groups, or sulfonyl groups, and, in certain embodiments, may have specific three-dimensional structural characteristics, and/or specific charge characteristics.

The term “substantial identity” or “substantially identical,” when referring to a nucleic acid or fragment thereof, indicates that, when optimally aligned with appropriate nucleotide insertions or deletions with another nucleic acid (or its complementary strand), there is nucleotide sequence identity in at least about 90%, and more preferably at least about 95%, 96%, 97%, 98% or 99% of the nucleotide bases, as measured by any well-known algorithm of sequence identity, such as FASTA, BLAST or GAP, as discussed below.

As applied to polypeptides, the term “substantial similarity” or “substantially similar” means that two peptide sequences, when optimally aligned, such as by the programs GAP or BESTFIT using default gap weights, share at least 90% sequence identity, even more preferably at least 95%, 98% or 99% sequence identity. Preferably, residue positions that are not identical differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent or degree of similarity may be adjusted upwards to correct for the conservative nature of the substitution. Means for making this adjustment are well known to those of skill in the art. See, e.g., Pearson (1994) Methods Mol. Biol. 24:307-331. Examples of groups of amino acids that have side chains with similar chemical properties include 1) aliphatic side chains: glycine, alanine, valine, leucine and isoleucine; 2) aliphatic-hydroxyl side chains: serine and threonine; 3) amide-containing side chains: asparagine and glutamine; 4) aromatic side chains: phenylalanine, tyrosine, and tryptophan; 5) basic side chains: lysine, arginine, and histidine; 6) acidic side chains: aspartate and glutamate, and 7) sulfur-containing side chains: cysteine and methionine. Preferred conservative amino acids substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al. (1992) Science 256:1443 45. A “moderately conservative” replacement is any change having a nonnegative value in the PAM250 log-likelihood matrix.

Sequence similarity for polypeptides is typically measured using sequence analysis software. Protein analysis software matches similar sequences using measures of similarity assigned to various substitutions, deletions and other modifications, including conservative amino acid substitutions. For instance, GCG software contains programs such as GAP and BESTFIT, which can be used with default parameters to determine sequence homology or sequence identity between closely related polypeptides, such as homologous polypeptides from different species of organisms or between a wild type protein and a mutein thereof. See, e.g., GCG Version 6.1. Polypeptide sequences also can be compared using FASTA with default or recommended parameters; a program in GCG Version 6.1. FASTA (e.g., FASTA2 and FASTA3) provides alignments and percent sequence identity of the regions of the best overlap between the query and search sequences (Pearson (2000) supra). Another preferred algorithm when comparing a sequence of the invention to a database containing a large number of sequences from different organisms is the computer program BLAST, especially BLASTP or TBLASTN, using default parameters. See, e.g., Altschul et al. (1990) J. Mol. Biol. 215:403 410 and (1997) Nucleic Acids Res. 25:3389 402.

By the phrase “therapeutically effective amount” is meant an amount that produces the desired effect for which it is administered. The exact amount will depend on the purpose of the treatment, the age and the size of a subject treated, the route of administration, and the like, and will be ascertainable by one skilled in the art using known techniques (see, for example, Lloyd (1999) The Art, Science and Technology of Pharmaceutical Compounding).

### Bioequivalents

The anti-hANGPTL3 antibodies and antibody fragments described herein encompass proteins having amino acid sequences that vary from those of the described mAbs, but that retain the ability to bind human ANGPTL3. Such variant mAbs and antibody fragments comprise one or more additions, deletions, or substitutions of amino acids when compared to parent sequence, but exhibit biological activity that is essentially equivalent to that of the described mAbs. Likewise, the anti-hANGPTL3 antibody-encoding DNA sequences described herein encompass sequences that comprise one or more additions, deletions, or substitutions of nucleotides when compared to the disclosed sequence, but that encode an anti-hANGPTL3 antibody or antibody fragment that is essentially bioequivalent to an anti-hANGPTL3 antibody or antibody fragment described herein. Examples of such variant amino acid and DNA sequences are discussed above.

Two antigen-binding proteins, or antibodies, are considered bioequivalent if, for example, they are pharmaceutical equivalents or pharmaceutical alternatives whose rate and extent of absorption do not show a significant difference when administered at the same molar dose under similar experimental conditions, either single does or multiple dose. Some antibodies will be considered equivalents or pharmaceutical alternatives, if they are equivalent in the extent of their absorption but not in their rate of absorption and yet may be considered bioequivalent, because such differences in the rate of absorption are intentional and are reflected in the labeling, are not essential to the attainment of effective body drug concentrations on, e.g., chronic use, and are considered medically insignificant for the particular drug product studied. In one embodiment, two antigen-binding proteins are bioequivalent if there are no clinically meaningful differences in their safety, purity, and potency.

In one embodiment, two antigen-binding proteins are bioequivalent if a patient can be switched one or more times between the reference product and the biological product without an expected increase in the risk of adverse effects, including a clinically significant change in immunogenicity, or diminished effectiveness, as compared to continued therapy without such switching.

In one embodiment, two antigen-binding proteins are bioequivalent if they both act by a common mechanism or mechanisms of action for the condition or conditions of use, to the extent that such mechanisms are known.

Bioequivalence may be demonstrated by in vivo and in vitro methods. Bioequivalence measures include, e.g., (a) an in vivo test in humans or other mammals, in which the concentration of the antibody or its metabolites is measured in blood, plasma, serum, or other biological fluid as a function of time; (b) an in vitro test that has been correlated with and is reasonably predictive of human in vivo bioavailability data; (c) an in vivo test in humans or other mammals in which the appropriate acute pharmacological effect of the antibody (or its target) is measured as a function of time; and (d) in a well-controlled clinical trial that establishes safety, efficacy, or bioavailability or bioequivalence of an antibody.

Bioequivalent variants of anti-hANGPTL3 antibodies of the invention may be constructed by, for example, making various substitutions of residues or sequences or deleting terminal or internal residues or sequences not needed for biological activity. For example, cysteine residues not essential for biological activity can be deleted or replaced with other amino acids to prevent formation of unnecessary or incorrect intramolecular disulfide bridges upon renaturation.

Antibodies that Bind Specifically to ANGPTL3

The pharmaceutical formulations of the present invention may comprise a human antibody, or an antigen-binding fragment thereof, that binds specifically to human angiopoietin-like protein 3 (ANGPTL3). Exemplary anti-human ANGPTL3 antibodies that may be included in the pharmaceutical formulations of the present invention are set forth in patent application publications U.S. Pat. No. 9,018,356B2, WO2008/073300, and U.S. Pat. No. 7,935,796, the disclosures of which are incorporated by reference in their entirety.

In certain embodiments, the anti-ANGPTL3 antibodies comprise HCVR/LCVR amino acid sequence pairs having SEQ ID NOs selected from the group consisting of 2/10 (“H4H1248P”), 18/26 (“H4H1250P”), 34/42 (“H4H1263S”), 50/58 (“H4H1268S”), 66/74 (“H4H1276S”), 82/90 (“H4H1279P”), 98/106 (“H4H1282P”), 114/122 (“H4H1292P”), 130/138 (“H4H1295P”), 146/154 (“H4H1296P”), and 180/188 (“H1M896N”).

According to certain embodiments of the present invention, the anti-human ANGPTL3 antibody, or antigen-binding fragment thereof, comprises a heavy chain complementary determining region (HCDR) 1 of SEQ ID NO: 68, an HCDR2 of SEQ ID NO: 70, and an HCDR3 of SEQ ID NO: 72. In certain embodiments, the anti-human ANGPTL3 antibody, or antigen-binding fragment thereof, comprises an HCVR of SEQ ID NO:66.

According to certain embodiments of the present invention, the anti-human ANGPTL3 antibody, or antigen-binding fragment thereof, comprises a light chain complementary determining region (LCDR) 1 of SEQ ID NO: 76, an LCDR2 of KAS, and an LCDR3 of SEQ ID NO: 80. In certain embodiments, the anti-human ANGPTL3 antibody, or antigen-binding fragment thereof, comprises an LCVR of SEQ ID NO: 74.

According to certain embodiments of the present invention, the anti-human ANGPTL3 antibody, or antigen-binding fragment thereof, comprises a HCVR having 90%, 95%, 98% or 99% sequence identity to SEQ ID NO: 66.

According to certain embodiments of the present invention, the anti-human ANGPTL3 antibody, or antigen-binding fragment thereof, comprises a LCVR having 90%, 95%, 98% or 99% sequence identity to SEQ ID NO: 74.

According to certain embodiments of the present invention, the anti-human ANGPTL3 antibody, or antigen-binding fragment thereof, comprises a HCVR comprising an amino acid sequence of SEQ ID NO: 66 having no more than 5 amino acid substitutions.

According to certain embodiments of the present invention, the anti-ANGPTL3 antibody, or antigen-binding fragment thereof, comprises a LCVR comprising an amino acid sequence of SEQ ID NO: 74 having no more than 2 amino acid substitutions.

Sequence identity may be measured by any method known in the art (e.g., GAP, BESTFIT, and BLAST).

The present invention also includes formulations comprising anti-ANGPTL3 antibodies, wherein the anti-ANGPTL3 antibodies comprise variants of any of the HCVR, LCVR and/or CDR amino acid sequences disclosed herein having one or more conservative amino acid substitutions. For example, the present invention includes formulations comprising anti-ANGPTL3 antibodies having HCVR, LCVR and/or CDR amino acid sequences with, e.g., 10 or fewer, 8 or fewer, 6 or fewer, 4 or fewer, etc. conservative amino acid substitutions relative to any of the HCVR, LCVR and/or CDR amino acid sequences disclosed herein.

In certain embodiments, the anti-ANGPTL3 antibody comprises a Fc region elected from the group consisting of human IgG1, IgG2, IgG3, and IgG4 isotypes.

The non-limiting, exemplary antibody used in the Examples herein is referred to as “H4H1276S” or “mAb1”. This antibody is also referred to in U.S. Pat. No. 9,018,356B2 as H4H1276S. mAb1 (H4H1276S) comprises an HCVR/LCVR amino acid sequence pair having SEQ ID NOs: 66/74, and HCDR1-HCDR2-HCDR3/LCDR1-LCDR2-LCDR3 domains represented by SEQ ID NOs: 68-70-72/SEQ ID NOs: 76-KAS-80.

The full length sequences of H4H1276S are the following:

Heavy chain sequence
(SEQ ID NO: 195)
EVQLVESGGGVIQPGGSLRLSCAASGFTFDDYAMNWVRQGPGKGLEWVSA
ISGDGGSTYYADSVKGRFTISRDNSKNSLYLQMNSLRAEDTAFFYCAKDL
RNTIFGVVIPDAFDIWGQGTMVTVSSASTKGPSVFPLAPCSRSTSESTAA
LGCLVKDYFPEPVTVSWNSGALTSGVHTFPAVLQSSGLYSLSSVVTVPSS
SLGTKTYTCNVDHKPSNTKVDKRVESKYGPPCPPCPAPEFLGGPSVFLFP
PKPKDTLMISRTPEVTCVVVDVSQEDPEVQFNWYVDGVEVHNAKTKPREE
QFNSTYRVVSVLTVLHQDWLNGKEYKCKVSNKGLPSSIEKTISKAKGQPR
EPQVYTLPPSQEEMTKNQVSLTCLVKGFYPSDIAVEWESNGQPENNYKTT
PPVLDSDGSFFLYSRLTVDKSRWQEGNVFSCSVMHEALHNHYTQKSLSLS
LGK
Light chain sequence
(SEQ ID NO: 196)
DIQMTQSPSTLSASVGDRVTITCRASQSIRSWLAWYQQKPGKAPKLLIYK
ASSLESGVPSRFSGSGSGTEFTLTISSLQPDDFATYYCQQYNSYSYTFGQ
GTKLEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC

The amount of antibody, or antigen-binding fragment thereof, contained within the pharmaceutical formulations of the present invention may vary depending on the specific properties desired of the formulations, as well as the particular circumstances and purposes for which the formulations are intended to be used. In certain embodiments, the pharmaceutical formulations are liquid formulations that may contain 5±0.75 mg/mL to 250±37.5 mg/ml of antibody; 10±1.5 mg/mL to 240±36 mg/ml of antibody; 20±3.0 mg/mL to 230±34.5 mg/mL of antibody; 25±3.75 mg/ml to 240±36 mg/ml of antibody; 50±7.5 mg/mL to 230±34.5 mg/ml of antibody; 60±9 mg/mL to 240±36 mg/ml of antibody; 70±10.5 mg/mL to 230±34.5 mg/mL of antibody; 80±12 mg/mL to 220±33 mg/ml of antibody; 90±13.5 mg/mL to 210±31.5 mg/ml of antibody; 100±15 mg/mL to 200±30 mg/mL of antibody; 110±16.5 mg/mL to 190±28.5 mg/mL of antibody; 120±18 mg/mL to 180±27 mg/ml of antibody; 130±19.5 mg/mL to 170±25.5 mg/ml of antibody; 140±21 mg/mL to 160±24 mg/ml of antibody; 150±22.5 mg/ml of antibody; or 175±26.25 mg/ml. For example, the formulations of the present invention may comprise about 5 mg/ml; about 10 mg/mL; about 15 mg/mL; about 20 mg/mL; about 25 mg/mL; about 30 mg/ml; about 35 mg/ml; about 40 mg/mL; about 45 mg/mL; about 50 mg/mL; about 55 mg/ml; about 60 mg/ml; about 65 mg/ml; about 70 mg/ml; about 75 mg/mL; about 80 mg/mL; about 85 mg/mL; about 90 mg/mL; about 95 mg/ml; about 100 mg/mL; about 105 mg/ml; about 110 mg/mL; about 115 mg/mL; about 120 mg/mL; about 125 mg/mL; about 130 mg/mL; about 135 mg/mL; about 140 mg/ml; about 145 mg/mL; about 150 mg/ml; about 155 mg/mL; about 160 mg/mL; about 165 mg/mL; about 170 mg/ml; about 175 mg/mL; about 180 mg/mL; about 185 mg/mL; about 190 mg/mL; about 195 mg/mL; about 200 mg/mL; about 205 mg/mL; about 210 mg/ml; about 215 mg/mL; about 220 mg/ml; about 225 mg/mL; about 230 mg/mL; about 235 mg/mL; about 240 mg/ml; about 245 mg/ml; or about 250 mg/mL of an antibody or an antigen-binding fragment thereof, that binds specifically to human ANGPTL3. In one embodiment, the formulation of the invention comprises about 150 mg/mL anti-human ANPTL3 antibody or antigen-binding fragment thereof.

### Excipients and pH

The pharmaceutical formulations of the present invention comprise one or more excipients. The term “excipient”, as used herein, means any non-therapeutic agent added to the formulation to provide a desired consistency, viscosity, or stabilizing effect.

In certain embodiments, the pharmaceutical formulation of the invention comprises at least one organic cosolvent in a type and in an amount that stabilizes the anti-hANGPTL3 antibody under conditions of rough handling or agitation, such as, e.g., vortexing. In some embodiments, what is meant by “stabilizes” is the prevention of the formation of more than 3% aggregated antibody of the total amount of antibody (on a molar basis) over the course of rough handling. In some embodiments, rough handling is vortexing a solution containing the antibody and the organic cosolvent for about 60 minutes or about 120 minutes.

In certain embodiments, the organic cosolvent is a non-ionic surfactant, such as an alkyl poly(ethylene oxide). Specific non-ionic surfactants that can be included in the formulations of the present invention include, e.g., polysorbates such as polysorbate 20, polysorbate 28, polysorbate 40, polysorbate 60, polysorbate 65, polysorbate 80, polysorbate 81, and polysorbate 85; poloxamers such as poloxamer 181, poloxamer 188, poloxamer 407; or polyethylene glycol (PEG). Polysorbate 20 is also known as TWEEN 20, sorbitan monolaurate and polyoxyethylenesorbitan monolaurate. Poloxamer 188 is also known as PLURONIC F68. In certain embodiments, the organic cosolvent comprised in the formulation of the invention is polysorbate 80.

The amount of non-ionic surfactant contained within the pharmaceutical formulations of the present invention may vary depending on the specific properties desired of the formulations, as well as the particular circumstances and purposes for which the formulations are intended to be used. In certain embodiments, the formulations may contain 0.01%±0.005% to 0.5%±0.25% surfactant. For example, the formulations of the present invention may comprise about 0.005%; about 0.01%; about 0.02%; about 0.03%; about 0.04%; about 0.05%; about 0.06%; about 0.07%; about 0.08%; about 0.09%; about 0.1%; about 0.11%; about 0.12%; about 0.13%; about 0.14%; about 0.15%; about 0.16%; about 0.17%; about 0.18%; about 0.19%; about 0.20%; about 0.21%; about 0.22%; about 0.23%; about 0.24%; about 0.25%; about 0.26%; about 0.27%; about 0.28%; about 0.29%; about 0.30%; about 0.35%; about 0.40%; about 0.45%; about 0.46%; about 0.47%; about 0.48%; about 0.49%; about 0.50%; about 0.55%; or about 0.575% polysorbate 20 or polysorbate 80. In certain embodiments, the formulation of the invention comprises about 0.1% (w/v) polysorbate 80.

The pharmaceutical formulations of the present invention may also comprise one or more stabilizers in a type and in an amount that stabilizes the anti-hANGPTL3 antibody under conditions of thermal stress. In some embodiments, what is meant by “stabilizes” is that some significant % of the antibody in a native conformation when the solution containing the antibody and the thermal stabilizer is kept at about 45° C. for up to about 28 days. In some embodiments, what is meant by “stabilizes” is wherein some insignificant % of the antibody is aggregated when the solution containing the antibody and the thermal stabilizer is kept at about 45° C. for up to about 28 days. As used herein, “native” means the major form of the antibody by size exclusion, which is generally an intact monomer of the antibody. The term “native” also refers to non-aggregated and non-degraded form of the antibody.

In certain embodiments, the thermal stabilizer is a sugar such as sucrose, the amount of which contained within the formulation can vary depending on the specific circumstances and intended purposes for which the formulation is used. In certain embodiments, the formulations may contain about 1% to about 15% sugar; about 2% to about 14% sugar; about 3% to about 13% sugar; about 4% to about 12% sugar; about 5% to about 12% sugar; about 6% to about 11% sugar; about 7% to about 10% sugar; about 8% to about 11% sugar; or about 9% to about 11% sugar. For example, the pharmaceutical formulations of the present invention may comprise 4%±0.8%; 5%±1%; 6%±1.2%; 7%±1.4%; 8%±1.6%; 9%±1.8%; 10%±2%; 11%±2.2%; 12%±2.4%; 13%±2.6%; or about 14%±2.8% sugar (e.g., sucrose). In certain embodiments, the formulation of the invention does not comprise sugar.

The pharmaceutical formulations of the present invention may also comprise a buffer or buffer system, which serves to maintain a stable pH and to help stabilize the anti-hANGPTL3 antibody. In some embodiments, what is meant by “stabilizes” is minimizing the amount of the antibody aggregated when the solution containing the antibody and the buffer is kept at about 45° C. for up to about 28 days. In some embodiments, what is meant by “stabilizes” is maximizing the amount of antibody is in its native conformation as determined by size exclusion chromatography when the solution containing the antibody and the buffer is kept at about 45° C. for up to about 28 days. By “native” or “native conformation”, what is meant is the antibody fraction that is not aggregated or degraded. This is generally determined by an assay that measures the relative size of the antibody entity, such as a size exclusion chromatographic assay. The non-aggregated and non-degraded antibody elutes at a fraction that equates to the native antibody, and is generally the main elution fraction. Aggregated antibody elutes at a fraction that indicates a size greater than the native antibody. Degraded antibody elutes at a fraction that indicates a size less than the native antibody.

In some embodiments, what is meant by “stabilizes” is wherein at least about 46% of the antibody is in its main charge form as determined by cation exchange chromatography when the solution containing the antibody and the buffer is kept at about 45° C. for up to about 28 days. By “main charge” or “main charge form”, what is meant is the fraction of antibody that elutes from an ion exchange resin in the main peak, which is generally flanked by more “basic” peaks on one side and more “acidic” peaks on the other side.

The pharmaceutical formulations of the present invention may have a pH of from about 5.2 to about 6.4. For example, the formulations of the present invention may have a pH of about 5.5; about 5.6; about 5.7; about 5.8; about 5.9; about 6.0; about 6.1; about 6.2; about 6.3; about 6.4; or about 6.5. In some embodiments, the pH is 6.0±0.4; 6.0±0.3; 6.0±0.2; 6.0±0.1; about 6.0; or 6.0.

In some embodiments, the buffer or buffer system comprises at least one buffer that has a buffering range that overlaps fully or in part the range of pH 5.5-7.4. In certain embodiments, the buffer comprises a histidine buffer. In certain embodiments, the histidine is present at a concentration of 5 mM±1 mM to 15 mM±3 mM; 6 mM±1.2 mM to 14 mM±2.8 mM; 7 mM±1.4 mM to 13 mM±2.6 mM; 8 mM±1.6 mM to 12 mM±2.4 mM; 9 mM±1.8 mM to 11 mM±2.2 mM; 10 mM±2 mM; or about 10 mM. In certain embodiments, the buffer system comprises histidine at 10 mM±2 mM, at a pH of 6.0±0.3.

The pharmaceutical formulations of the present invention may also comprise one or more excipients that serve to maintain a reduced viscosity or to lower the viscosity of formulations containing a high concentration of anti-ANGPTL3 antibody drug substance (e.g., generally about 150 mg/ml of antibody). In certain embodiments, the at least one viscosity modifier is selected from the group consisting of: Arginine-HCl, Sodium Chloride, Histidine-HCl, Sodium Acetate (pH 5), Calcium Chlorise, Magnesium Chloride, Calcium Acetate, and Magnesium Acetate. In certain embodiments, the formulations of the invention comprise Arginine HCl.

In certain embodiments, the pharmaceutical formulation includes at least one amino acid. In certain embodiments, the amino acid is proline, and the pharmaceutical formulation of the present invention contains proline, preferably as L-proline, at a concentration of 1%, 1.5%, 2%, 2.5%, 3%, 3.5%, 4%, 4.5% or 5%. In some embodiments, the formulation comprises proline in an amount sufficient to maintain the viscosity of the liquid formulation at less than 20±3 cPoise, less than 15±2.25 cPoise, or less than 11±1.65 cPoise. In some embodiments, the formulation comprises proline in an amount sufficient to maintain the viscosity at or below 15±2.25 cPoise. In certain embodiments, formulations may contain about 1% to about 5% proline; about 2% to about 4% proline; or about 3% proline. For example, the pharmaceutical formulations of the present invention may comprise 1%±0.2%; 1.5%±0.3%; 2%±0.4%; 2.5%±0.5%; 3%±0.6%; 3.5%±0.7%; 4%±0.8%; 4.5%±0.9%; or about 5%±1% proline.

During the antibody purification process it may be desired or necessary to exchange one buffer for another to achieve appropriate excipient concentrations, antibody concentration, pH, etc. Buffer exchange can be accomplished, e.g., by ultrafiltration/diafiltration (UF/DF) using, e.g., a semi-permeable tangential flow filtration membrane. Use of such techniques, however, has the potential to cause the Gibbs-Donnan effect [Bolton et al., 2011, Biotechnol. Prog. 27(1): 140-152]. The buildup of positive charge on the product side of the membrane during protein concentration is counterbalanced electrically by the preferential movement of positive ions to the opposite side of the membrane. The potential consequence of this phenomenon is that the final concentrations of certain components (e.g., histidine, L-proline, etc.) may be lower than the intended target concentrations of these components due to the electrostatic repulsion of positively charged diafiltration buffer excipients to the positively charged antibody protein during the UF/DF step. Thus, the present invention includes formulations in which the concentration of, e.g., histidine and/or L-proline vary from the recited amounts or ranges herein due to the Gibbs-Donnan effect.

Volume exclusion describes the behavior of highly concentrated samples in which a significant portion of the total volume of the solution is taken up by the solute, especially large molecules such as proteins, excluding the solvent from this space. This then decreases the total volume of solvent available for other solutes to be dissolved in, which may result in unequal partition across the ultrafiltration membrane. Thus, the present invention includes formulations in which the concentration of, e.g., histidine and/or L-proline may vary from the recited amounts or ranges herein due to the volume exclusion effect.

During the manufacture of the formulations of the present invention, variations in the composition of the formulation may occur. These variations may include the concentration of the active ingredient, the concentration of the excipients, and/or the pH of the formulation. Because changes in any of these parameters could potentially impact the stability or potency of the drug product, proven acceptable range (PAR) studies were conducted to assess whether variations in the composition, within the defined ranges, would impact the stability or potency of the antibody. Accordingly, the present invention includes formulations comprising anti-ANGPTL3 antibodies that are stable and retain potency with up to 50% variation in the excipient concentration. For example, included herein are anti-ANGPTL3 antibody formulations, wherein stability and potency of said formulations is unaffected by ±10%, ±20%, ±30%, ±40% or ±50% variation in the concentration of antibody, histidine, Arginine-HCl, and/or polysorbate.

### Stability and Viscosity of the Pharmaceutical Formulations

The pharmaceutical formulations of the present invention typically exhibit high levels of stability. The term “stable”, as used herein in reference to the pharmaceutical formulations, means that the antibodies within the pharmaceutical formulations retain an acceptable degree of chemical structure or biological function after storage under defined conditions. A formulation may be stable even though the antibody contained therein does not maintain 100% of its chemical structure or biological function after storage for a defined amount of time. Under certain circumstances, maintenance of about 90%, about 95%, about 96%, about 97%, about 98% or about 99% of an antibody's structure or function after storage for a defined amount of time may be regarded as “stable”.

Stability can be measured, inter alia, by determining the percentage of native antibody that remains in the formulation after storage for a defined amount of time at a defined temperature. The percentage of native antibody can be determined by, inter alia, size exclusion chromatography (e.g., size exclusion ultra performance liquid chromatography [SE-UPLC]), such that native means non-aggregated and non-degraded. An “acceptable degree of stability”, as that phrase is used herein, means that at least 90% of the native form of the antibody can be detected in the formulation after storage for a defined amount of time at a given temperature. In certain embodiments, at least about 90%, 91%, 92%, 93%, 94%, 95%, 96%, 97%, 98%, 99% or 100% of the native form of the antibody can be detected in the formulation after storage for a defined amount of time at a defined temperature. The defined amount of time after which stability is measured can be at least 14 days, at least 28 days, at least 1 month, at least 2 months, at least 3 months, at least 4 months, at least 5 months, at least 6 months, at least 7 months, at least 8 months, at least 9 months, at least 10 months, at least 11 months, at least 12 months, at least 18 months, at least 24 months, or more. The defined temperature at which the pharmaceutical formulation may be stored when assessing stability can be any temperature from about −80° C. to about 45° C., e.g., storage at about −80° C., about −30° C., about −20° C., about 0° C., about 4°−8° C., about 5° C., about 25° C., about 35° C., about 37° C., or about 45° C. For example, a pharmaceutical formulation may be deemed stable if after 6 months of storage at 5° C., greater than about 95%, 96%, 97% or 98% of native antibody is detected by SE-UPLC. A pharmaceutical formulation may also be deemed stable if after 6 months of storage at 25° C., greater than about 95%, 96%, 97% or 98% of native antibody is detected by SE-UPLC. A pharmaceutical formulation may also be deemed stable if after 28 days of storage at 45° C., greater than about 89%, 90%, 91%, 92%, 93%, 94%, 95% or 96% of native antibody is detected by SE-UPLC. A pharmaceutical formulation may also be deemed stable if after 12 months of storage at −20° C., greater than about 96%, 97%, or 98% of native antibody is detected by SE-UPLC. A pharmaceutical formulation may also be deemed stable if after 12 months of storage at −30° C., greater than about 96%, 97% or 98% of native antibody is detected by SE-UPLC. A pharmaceutical formulation may also be deemed stable if after 12 months of storage at −80° C., greater than about 96%, 97% or 98% of native antibody is detected by SE-UPLC.

Stability can be measured, inter alia, by determining the percentage of antibody that forms in an aggregate within the formulation after storage for a defined amount of time at a defined temperature, wherein stability is inversely proportional to the percent aggregate that is formed. The percentage of aggregated antibody can be determined by, inter alia, size exclusion chromatography (e.g., size exclusion ultra performance liquid chromatography [SE-UPLC]). An “acceptable degree of stability”, as that phrase is used herein, means that at most 5% of the antibody is in an aggregated form (also denoted as the high molecular weight-HMW-form) detected in the formulation after storage for a defined amount of time at a given temperature. In certain embodiments an acceptable degree of stability means that at most about 5%, 4%, 3%, 2%, 1%, 0.5%, or 0.1% of the antibody can be detected in an aggregate in the formulation after storage for a defined amount of time at a given temperature. The defined amount of time after which stability is measured can be at least 2 weeks, at least 28 days, at least 1 month, at least 2 months, at least 3 months, at least 4 months, at least 5 months, at least 6 months, at least 7 months, at least 8 months, at least 9 months, at least 10 months, at least 11 months, at least 12 months, at least 18 months, at least 24 months, or more. The temperature at which the pharmaceutical formulation may be stored when assessing stability can be any temperature from about −80° C. to about 45° C., e.g., storage at about −80° C., about −30° C., about −20° C., about 0° C., about 4°−8° C., about 5° C., about 25° C., about 35° C., about 37° C. or about 45° C. For example, a pharmaceutical formulation may be deemed stable if after 12 months of storage at 5° C., less than about 2%, 1%, 0.5%, or 0.1% of the antibody is detected in an aggregated form. A pharmaceutical formulation may also be deemed stable if after three months of storage at 25° C., less than about 4%, 3%, 2%, 1%, 0.5%, or 0.1% of the antibody is detected in an aggregated form. A pharmaceutical formulation may also be deemed stable if after 28 days of storage at 45° C., less than about 10%, 9%, 8%, 7%, 6%, 5%, 4%, 3%, 2%, 1%, or 0.5%, of the antibody is detected in an aggregated form. A pharmaceutical formulation may also be deemed stable if after three months of storage at −20° C., −30° C., or −80° C. less than about 3%, 2%, 1%, 0.5%, or 0.1% of the antibody is detected in an aggregated form.

Stability can be measured, inter alia, by determining the percentage of antibody that migrates in a more acidic fraction during ion exchange (“acidic form”) than in the main fraction of antibody (“main charge form”), wherein stability is inversely proportional to the fraction of antibody in the acidic form. While not wishing to be bound by theory, deamidation of the antibody may cause the antibody to become more negatively charged and thus more acidic relative to the non-deamidated antibody (see, e.g., Robinson, N., Protein Deamidation, PNAS, Apr. 16, 2002, 99(8):5283-5288). The percentage of “acidified” antibody can be determined by, inter alia, ion exchange chromatography (e.g., cation exchange ultra performance liquid chromatography [CEX-UPLC]). An “acceptable degree of stability”, as that phrase is used herein, means that at most 45% of the antibody is in a more acidic form detected in the formulation after storage for a defined amount of time at a defined temperature. In certain embodiments an acceptable degree of stability means that at most about 40%, 35%, 30%, 25%, 20%, 15%, 10%, 5%, 4%, 3%, 2%, 1%, 0.5%, or 0.1% of the antibody can be detected in an acidic form in the formulation after storage for a defined amount of time at a given temperature. In one embodiment, an acceptable degree of stability means that less than 30%, 25%, 20%, 15%, 10%, 5%, 4%, 3%, 2%, 1%, 0.5%, or 0.1% of the antibody can be detected in an acidic form in the formulation after storage for a defined amount of time at a given temperature. The defined amount of time after which stability is measured can be at least 2 weeks, at least 28 days, at least 1 month, at least 2 months, at least 3 months, at least 4 months, at least 5 months, at least 6 months, at least 7 months, at least 8 months, at least 9 months, at least 10 months, at least 11 months, at least 12 months, at least 18 months, at least 24 months, or more. The temperature at which the pharmaceutical formulation may be stored when assessing stability can be any temperature from about −80° C. to about 45° C., e.g., storage at about-80° C., about −30° C., about −20° C., about 0° C., about 4°-8° C., about 5° C., about 25° C., or about 45° C. For example, a pharmaceutical formulation may be deemed stable if after three months of storage at −80° C., −30° C., or −20° C. less than about 30%, 29%, 28%, 27%, 26%, 25%, 24%, 23%, 22%, 21%, 20%, 19%, 18%, 17%, 16%, 15%, 14%, 13%, 12%, 10%, 9%, 8%, 7%, 6%, 5%, 4%, 3%, 2%, 1%, 0.5% or 0.1% of the antibody is in a more acidic form. A pharmaceutical formulation may also be deemed stable if after six months of storage at 5° C., less than about 32%, 31%, 30%, 29%, 28%, 27%, 26%, 25%, 24%, 23%, 22%, 21%, 20%, 19%, 18%, 17%, 16%, 15%, 14%, 13%, 12%, 10%, 9%, 8%, 7%, 6%, 5%, 4%, 3%, 2%, 1%, 0.5% or 0.1% of the antibody is in a more acidic form. A pharmaceutical formulation may also be deemed stable if after six months of storage at 25° C., less than about 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 10%, 9%, 8%, 7%, 6%, 5%, 4%, 3%, 2%, 1%, 0.5% or 0.1% of the antibody is in a more acidic form. A pharmaceutical formulation may also be deemed stable if after 28 days of storage at 45° C., less than about 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 10%, 9%, 8%, 7%, 6%, 5%, 4%, 3%, 2%, 1%, 0.5% or 0.1% of the antibody can be detected in a more acidic form.

Other methods may be used to assess the stability of the formulations of the present invention such as, e.g., differential scanning calorimetry (DSC) to determine thermal stability, controlled agitation to determine mechanical stability, and absorbance at about 350 nm or about 405 nm to determine solution turbidities. For example, a formulation of the present invention may be considered stable if, after 6 or more months of storage at about 5° C. to about 25° C., the change in OD₄₀₅ of the formulation is less than about 0.05 (e.g., 0.04, 0.03, 0.02, 0.01, or less) from the OD₄₀₅ of the formulation at time zero.

Measuring the biological activity or binding affinity of the antibody to its target may also be used to assess stability. For example, a formulation of the present invention may be regarded as stable if, after storage at e.g., 5° C., 25° C., 45° C., etc. for a defined amount of time (e.g., 1 to 12 months), the anti-ANGPTL3 antibody contained within the formulation binds to ANGPTL3 with an affinity that is at least 90%, 95%, or more of the binding affinity of the antibody prior to said storage. Binding affinity may be determined by e.g., ELISA or surface plasmon resonance. Biological activity may be determined by an ANGPTL3 activity assay, such as e.g., contacting a cell that expresses ANGPTL3 with the formulation comprising the anti-ANGPTL3 antibody. The binding of the antibody to such a cell may be measured directly, such as e.g., via FACS analysis. Alternatively, the downstream activity of the ANGPTL3 system may be measured in the presence of the antibody, and compared to the activity of the ANGPTL3 system in the absence of antibody. In some embodiments, the ANGPTL3 may be endogenous to the cell. In other embodiments, the ANGPTL3 may be ectopically expressed in the cell.

Additional methods for assessing the stability of an antibody in formulation are demonstrated in the Examples presented below.

The liquid pharmaceutical formulations of the present invention may, in certain embodiments, exhibit low to moderate levels of viscosity. “Viscosity” as used herein may be “kinematic viscosity” or “absolute viscosity”. “Kinematic viscosity” is a measure of the resistive flow of a fluid under the influence of gravity. When two fluids of equal volume are placed in identical capillary viscometers and allowed to flow by gravity, a viscous fluid takes longer than a less viscous fluid to flow through the capillary. For example, if one fluid takes 200 seconds to complete its flow and another fluid takes 400 seconds, the second fluid is twice as viscous as the first on a kinematic viscosity scale. “Absolute viscosity”, sometimes called dynamic or simple viscosity, is the product of kinematic viscosity and fluid density (Absolute Viscosity=Kinematic Viscosity×Density). The dimension of kinematic viscosity is L²/T where L is a length and T is a time. Commonly, kinematic viscosity is expressed in centistokes (cSt). The SI unit of kinematic viscosity is mm²/s, which is 1 cSt. Absolute viscosity is expressed in units of centipoise (cP). The SI unit of absolute viscosity is the milliPascal-second (mPa-s), where 1 cP=1 mPa-s.

As used herein, a low level of viscosity, in reference to a fluid formulation of the present invention, will exhibit an absolute viscosity of less than about 20 cPoise (cP). For example, a fluid formulation of the invention will be deemed to have “low viscosity”, if, when measured using standard viscosity measurement techniques, the formulation exhibits an absolute viscosity of about 20 cP, about 19 cP, about 18 cP, about 15 cP, about 12 cP, about 10 cP, about 9 cP, about 8 cP, or less. As used herein, a moderate level of viscosity, in reference to a fluid formulation of the present invention, will exhibit an absolute viscosity of between about 35 cP and about 20 cP. For example, a fluid formulation of the invention will be deemed to have “moderate viscosity”, if when measured using standard viscosity measurement techniques, the formulation exhibits an absolute viscosity of about 34 cP, about 33 cP, about 32 cP, about 31 cP, about 30 cP, about 29 cP, about 28 cP, about 27 cP, about 26 cP, about 25 cP, about 24 cP, about 23 cP, about 22 cP, about 21 cP, about 20 cP, about 19 cP, 18 cP, about 17 cP, about 16 cP, or about 15.1 cP.

As illustrated in the examples below, the present inventors have made the surprising discovery that low viscosity liquid formulations comprising high concentrations of an anti-human ANGPTL3 antibody (e.g., from about 50 mg/ml up to at least 250 mg/mL) can be obtained by formulating the antibody with proline from about 1% to about 5% and without the need for a stabilizer like sucrose. Such formulations are stable to stress during handling and to storage at temperatures ranging from 45° C. to −80° C. (shown herein) and show viscosity below about 15 cP.

### Exemplary Formulations

According to one aspect of the present invention, the pharmaceutical formulation is a stable, low viscosity, generally physiologically isotonic liquid formulation, which comprises: (i) a human antibody or antigen-binding fragment thereof that specifically binds to human ANGPTL3 (e.g., H4H1276S), at a concentration of about 25 to about 250 mg/mL; (ii) a buffer system that provides sufficient buffering at about pH 6.0±0.3; (iii) an organic cosolvent, which protects the structural integrity of the antibody; and (iv) a viscosity modifier that is a viscosity-reducing excipient. According to another aspect of the present invention, the pharmaceutical formulation is a stable, low viscosity, generally physiologically isotonic liquid formulation, which comprises: (i) a human antibody or antigen-binding fragment thereof that specifically binds to human ANGPTL3 (e.g., H4H1276S), at a concentration of about 25 to about 250 mg/mL; (ii) a buffer system that provides sufficient buffering at about pH 6.0±0.3; (iii) an organic cosolvent, which protects the structural integrity of the antibody; (iv) a viscosity modifier that is a viscosity-reducing excipient; and (iv) an amino acid, which serves to keep the viscosity manageable for injection in a convenient volume for subcutaneous administration.

According to one embodiment, the stable, low-viscosity pharmaceutical formulation comprises: (i) a human IgG4 antibody that specifically binds to human ANGPTL3, and which comprises an HCDR1 of SEQ ID NO: 68, an HCDR2 of SEQ ID NO: 70, an HCDR3 of SEQ ID NO: 72, an LCDR1 of SEQ ID NO: 76, an LCDR2 of KAS, and an LCDR3 of SEQ ID NO: 80, at a concentration of 25 mg/ml±7.5 mg/mL; (ii) histidine at 10 mM±2 mM, which buffers at pH 6.0±0.3; (iii) polysorbate 80 at 0.1% w/v±0.05% w/v; (iv) 70±5 mM Arginine-HCl; and (v) L-proline at about 3% (w/v)±0.6%. In another embodiment, the antibody comprises a HCVR/LCVR comprising an amino acid sequence pair of SEQ ID NOs: 66/74.

According to another embodiment, the stable, low-viscosity pharmaceutical formulation comprises (i) a human IgG4 antibody that specifically binds to human ANGPTL3, and which comprises an HCDR1 of SEQ ID NO: 68, an HCDR2 of SEQ ID NO: 70, an HCDR3 of SEQ ID NO: 72, an LCDR1 of SEQ ID NO: 76, an LCDR2 of KAS, and an LCDR3 of SEQ ID NO: 80, at a concentration of 50 mg/ml±7.5 mg/ml; (ii) 10±2 mM histidine; (iii) 0.1%±0.05% (w/v) polysorbate 80; (iv) 3%±0.6% proline; and (v) 70±5 mM Arginine-HCl, at a pH of 6.0±0.3. In another embodiment, the antibody comprises a HCVR/LCVR comprising an amino acid sequence pair of SEQ ID NOs: 66/74.

According to another embodiment, the stable, low-viscosity pharmaceutical formulation comprises (i) a human IgG4 antibody that specifically binds to human ANGPTL3, and which comprises an HCDR1 of SEQ ID NO: 68, an HCDR2 of SEQ ID NO: 70, an HCDR3 of SEQ ID NO: 72, an LCDR1 of SEQ ID NO: 76, an LCDR2 of KAS, and an LCDR3 of SEQ ID NO: 80, at a concentration of 100±15 mg/mL; (ii) 10±2 mM histidine; (iii) 0.1%±0.05% (w/v) (w/v) polysorbate 80; (iv) 3%±0.6% proline; and (v) 70±5 mM Arginine-HCl, at a pH of 6.0±0.3. In another embodiment, the antibody comprises a HCVR/LCVR comprising an amino acid sequence pair of SEQ ID NOs: 66/74.

According to another embodiment, the stable, low-viscosity pharmaceutical formulation comprises (i) a human IgG4 antibody that specifically binds to human ANGPTL3, and which comprises an HCDR1 of SEQ ID NO: 68, an HCDR2 of SEQ ID NO: 70, an HCDR3 of SEQ ID NO: 72, an LCDR1 of SEQ ID NO: 76, an LCDR2 of KAS, and an LCDR3 of SEQ ID NO: 80, at a concentration of 150±22.5 mg/ml; (ii) 10±2 mM histidine; (iii) 0.1%±0.05% (w/v) polysorbate 80; (iv) 70±5 mM Arginine-HCl; and (v) 3%±0.6% proline, at a pH of 6.0±0.3. In another embodiment, the antibody comprises a HCVR/LCVR comprising an amino acid sequence pair of SEQ ID NOs: 66/74. In certain embodiments of the formulations disclosed herein, the viscosity is less than about 20 cPoise; in further embodiments, the viscosity of the formulation is less than about 15 cPoise.

According to another embodiment, the stable, low-viscosity pharmaceutical formulation comprises (i) a human IgG4 antibody that specifically binds to human ANGPTL3, and which comprises an HCDR1 of SEQ ID NO: 68, an HCDR2 of SEQ ID NO: 70, an HCDR3 of SEQ ID NO: 72, an LCDR1 of SEQ ID NO: 76, an LCDR2 of KAS, and an LCDR3 of SEQ ID NO: 80, at a concentration of 175±26.25 mg/ml; (ii) 10±2 mM histidine; (iii) 0.1%±0.05% (w/v) polysorbate 80; (iv) 70±5 mM Arginine-HCl; and (v) 3%±0.6% proline, at a pH of 6.0±0.3. In another embodiment, the antibody comprises a HCVR/LCVR comprising an amino acid sequence pair of SEQ ID NOs: 66/74.

According to another embodiment, the stable, low-viscosity pharmaceutical formulation comprises (i) a human IgG4 antibody that specifically binds to human ANGPTL3, and which comprises an HCDR1 of SEQ ID NO: 68, an HCDR2 of SEQ ID NO: 70, an HCDR3 of SEQ ID NO: 72, an LCDR1 of SEQ ID NO: 76, an LCDR2 of KAS, and an LCDR3 of SEQ ID NO: 80, at a concentration of 200±30.00 mg/ml; (ii) 10±2 mM histidine; (iii) 0.1%±0.05% (w/v) polysorbate 80; (iv) 70±5 mM Arginine-HCl; and (v) 3%±0.6% proline, at a pH of 6.0±0.3. In another embodiment, the antibody comprises a HCVR/LCVR comprising an amino acid sequence pair of SEQ ID NOs: 66/74.

Additional non-limiting examples of pharmaceutical formulations encompassed by the present invention are set forth elsewhere herein, including the working Examples presented below.

### Containers and Methods of Administration

The pharmaceutical formulations of the present invention may be contained within any container suitable for storage of medicines and other therapeutic compositions. For example, the pharmaceutical formulations may be contained within a sealed and sterilized plastic or glass container having a defined volume such as a vial, ampule, syringe, cartridge, or bottle. Different types of vials can be used to contain the formulations of the present invention including, e.g., clear and opaque (e.g., amber) glass or plastic vials. Likewise, any type of syringe can be used to contain or administer the pharmaceutical formulations of the present invention.

The pharmaceutical formulations of the present invention may be contained within “normal tungsten” syringes or “low tungsten” syringes. As will be appreciated by persons of ordinary skill in the art, the process of making glass syringes generally involves the use of a hot tungsten rod that functions to pierce the glass thereby creating a hole from which liquids can be drawn and expelled from the syringe. This process results in the deposition of trace amounts of tungsten on the interior surface of the syringe. Subsequent washing and other processing steps can be used to reduce the amount of tungsten in the syringe. As used herein, the term “normal tungsten” means that the syringe contains greater than or equal to 500 parts per billion (ppb) of tungsten. The term “low tungsten” means that the syringe contains less than 500 ppb of tungsten. For example, a low tungsten syringe, according to the present invention, can contain less than about 490, 480, 470, 460, 450, 440, 430, 420, 410, 390, 350, 300, 250, 200, 150, 100, 90, 80, 70, 60, 50, 40, 30, 20, 10 or fewer ppb of tungsten.

The rubber plungers used in syringes, and the rubber stoppers used to close the openings of vials, may be coated to prevent contamination of the medicinal contents of the syringe or vial, or to preserve their stability. Thus, pharmaceutical formulations of the present invention, according to certain embodiments, may be contained within a syringe that comprises a coated plunger, or within a vial that is sealed with a coated rubber stopper. For example, the plunger or stopper may be coated with a fluorocarbon film. Examples of coated stoppers or plungers suitable for use with vials and syringes containing the pharmaceutical formulations of the present invention are mentioned in, e.g., U.S. Pat. Nos. 4,997,423; 5,908,686; 6,286,699; 6,645,635; and 7,226,554, the contents of which are incorporated by reference herein in their entireties. Particular exemplary coated rubber stoppers and plungers that can be used in the context of the present invention are commercially available under the tradename “FluroTec®”, available from West Pharmaceutical Services, Inc. (Lionville, PA). FluroTec® is an example of a flurocarbon coating used to minimize or prevent drug product from adhering to the rubber surfaces.

According to certain embodiments of the present invention, the pharmaceutical formulations may be contained within a low tungsten syringe that comprises a fluorocarbon-coated plunger.

The pharmaceutical formulations can be administered to a patient by parenteral routes such as injection (e.g., subcutaneous, intravenous, intramuscular, intraperitoneal, etc.) or percutaneous, mucosal, nasal, pulmonary or oral administration. Numerous reusable pen or autoinjector delivery devices can be used to subcutaneously deliver the pharmaceutical formulations of the present invention. Examples include, but are not limited to AUTOPEN™ (Owen Mumford, Inc., Woodstock, UK), DISETRONIC™ pen (Disetronic Medical Systems, Bergdorf, Switzerland), HUMALOG MIX 75/25™ pen, HUMALOG™ pen, HUMALIN 70/30™ pen (Eli Lilly and Co., Indianapolis, IN), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, NJ), OPTIPEN™ OPTIPEN PRO™, OPTIPEN STARLET™, and OPTICLIK™ (sanofi-aventis, Frankfurt, Germany). Examples of disposable pen or autoinjector delivery devices having applications in subcutaneous delivery of a pharmaceutical composition of the present invention include, but are not limited to the SOLOSTAR™ pen (sanofi-aventis), the FLEXPEN™ (Novo Nordisk), and the KWIKPEN™ (Eli Lilly), the SURECLICK™ Autoinjector (Amgen, Thousand Oaks, CA), the PENLET™ (Haselmeier, Stuttgart, Germany), the EPIPEN (Dey, L. P.), and the HUMIRA™ Pen (Abbott Labs, Abbott Park, IL).

The use of a microinfusor to deliver the pharmaceutical formulations of the present invention is also contemplated herein. As used herein, the term “microinfusor” means a subcutaneous delivery device designed to slowly administer large volumes (e.g., up to about 2.5 mL or more) of a therapeutic formulation over a prolonged period of time (e.g., about 10, 15, 20, 25, 30 or more minutes). See, e.g., U.S. Pat. Nos. 6,629,949; 6,659,982; and Meehan et al., J. Controlled Release 46:107-116 (1996). Microinfusors are particularly useful for the delivery of large doses of therapeutic proteins contained within high concentration (e.g., about 100, 125, 150, 175, 200 or more mg/mL) or viscous solutions.

In certain embodiments, the stable liquid pharmaceutical formulation of any of the preceding aspects is contained in a sterile glass vial and is administered as an IV infusion.

In one embodiment, the container is a 20 mL type 1 clear borosilicate glass vial. In certain embodiments, the container is a 2 mL or a 3 mL type 1 borosilicate glass vial with a FluroTec® coated 4432/50 butyl rubber stopper.

In one embodiment, the liquid pharmaceutical formulation of the present invention comprising about 25 mg/mL or 50 mg/ml of mAb1 is administered intravenously and may be contained in a glass vial.

In certain embodiments, the present invention provides an autoinjector comprising any of the liquid formulations described herein. In some embodiments, the present invention provides an autoinjector comprising a stable liquid formulation comprising about 50 mg/mL, about 100 mg/mL, about 150 mg/mL or about 175 mg/mL of mAb1, about 10 mM of histidine, at pH of about 6.0, about 70 mM Arginine-HCl, about 3% proline and about 0.1% polysorbate 80.

In certain embodiments, the present invention provides a pre-filled syringe comprising any of the liquid formulations described herein. In some embodiments, the present invention provides a prefilled syringe comprising a stable liquid formulation comprising about 50 mg/mL, about 100 mg/mL, about 150 mg/mL or about 175 mg/mL of mAb1, about 10 mM of histidine, at pH of about 6.0, about 70 mM Arginine-HCl, about 3% proline and about 0.1% polysorbate 80. In certain embodiments, the syringe is a 1 mL or 2.25 mL long glass syringe filled with a 27-gauge thin wall needle, a fluorocarbon coated rubber plunger and a rubber needle shield.

In one embodiment, a liquid pharmaceutical formulation containing about 175 mg/ml±26.25 mg/ml mAb1 is administered subcutaneously in a volume of approximately up to 2 mL in a prefilled syringe. In certain embodiments, the syringe is a 1 mL or 2.25 mL long glass syringe filled with a 27-gauge thin wall needle, a fluorocarbon coated rubber plunger and a rubber needle shield. In one embodiment, the syringe is an OMPI 1 mL long glass syringe fitted with a 27-gauge needle, a FM27 rubber needle shield, and a FLUROTEC® coated 4023/50 rubber plunger.

In one embodiment, the liquid pharmaceutical formulation containing about 150 mg/mL±22.5 mg/mL mAb1 is administered subcutaneously in a volume of about 1 to about 2 mL in a pre-filled syringe. In one embodiment, the syringe is a 1 mL or 2.25 mL long glass syringe filled with a 27-gauge thin wall needle, a fluorocarbon coated rubber plunger and a rubber needle shield. In one embodiment, the syringe is an OMPI 1 mL long glass syringe fitted with a 27-gauge needle, a FM27 rubber needle shield, and a FLUROTEC® coated 4023/50 rubber plunger.

### Therapeutic Uses of the Pharmaceutical Formulations

The pharmaceutical formulations of the present invention are useful, inter alia, for the treatment, prevention, or amelioration of any disease or disorder associated with ANGPTL3 activity, including diseases or disorders mediated by ANGPTL3. The disease or disorder treatable using the formulations of the invention is any disease or condition which is improved, ameliorated, inhibited, or prevented, or its occurrence rate reduced, compared to that without anti-hANGPTL3 antibody treatment (e.g., ANGPTL3-mediated diseases or disorders), by removing, inhibiting, reducing, or otherwise interfering with, ANGPTL3 activity.

Examples of diseases or disorders treatable using the formulations of the invention include, but are not limited to, those involving lipid metabolism, such as hyperlipidemia, hyperlipoproteinemia and dyslipidemia, including atherogenic dyslipidemia, diabetic dyslipidemia, hypertriglyceridemia, including severe hypertriglyceridemia with TG>1000 mg/dL, hypercholesterolemia, chylomicronemia, mixed dyslipidemia (obesity, metabolic syndrome, diabetes, etc.), lipodystrophy, lipoatrophy, and the like, which are caused by, for example, decreased LPL activity and/or LPL deficiency, decreased LDL receptor (LDLR) activity and/or LDL receptor deficiency (e.g., homozygous familial hypercholesterolemia with LDLR^{-/-}), altered ApoC2, ApoE deficiency, increased ApoB, increased production and/or decreased elimination of very low-density lipoprotein (VLDL), certain drug treatment (e.g., glucocorticoid treatment-induced dyslipidemia), any genetic predisposition, diet, lifestyle, and the like. The formulations of the invention can also prevent or treat diseases or disorders associated with or resulting from hyperlipidemia, hyper-lipoproteinemia, and/or dyslipidemia, including, but not limited to, cardiovascular diseases or disorders, such as atherosclerosis, aneurysm, hypertension, angina, stroke, cerebrovascular diseases, congestive heart failure, coronary artery diseases, myocardial infarction, peripheral vascular diseases, and the like; acute pancreatitis; nonalcoholic steatohepatitis (NASH); blood sugar disorders, such as diabetes; obesity, and the like.

Other examples of diseases or disorders treatable using the formulations of the invention include cancer/tumor as well as non-neoplastic angiogenesis-associated diseases or disorders, including ocular angiogenic diseases or disorders, such as age-related macular degeneration, central retinal vein occlusion or branch retinal vein occlusion, diabetic retinopathy, retinopathy of prematurity, and the like, inflammatory diseases or disorders, such as arthritis, rheumatoid arthritis (RA), psoriasis, and the like.

### EXAMPLES

The following examples are presented so as to provide those of ordinary skill in the art with a complete disclosure and description of how to make and use the methods and compositions of the invention and are not intended to limit the scope of what the inventors regard as their invention. Efforts have been made to ensure accuracy with respect to numbers used (e.g., amounts, temperature, etc.), but some experimental errors and deviations should be accounted for. Unless indicated otherwise, parts are parts by mole, molecular weight is average molecular weight, temperature is in degrees Centigrade, and pressure is at or near atmospheric pressure.

### Example 1: Development of an Anti-ANGPTL3 Antibody Formulation

For later stage clinical development, both IV and SC administrations of higher doses were planned. Therefore, formulation development studies were conducted with the goal of developing a 150 mg/mL liquid formulation that can be used for either IV or SC injection. A high concentration liquid formulation is required for delivering doses of H4H1276S, up to 450 mg, with one or two SC injections. For IV administration, a high concentration liquid formulation is also advantageous, because it permits addition of a smaller volume of DP to the IV infusion bag. This supports the use of doses as high as 15 mg of H4H1276S per kg patient weight.

Initial formulation development activities for the lyophilized H4H1276S formulation were conducted at a low protein concentration (5-50 mg/mL H4H1276S) and included an assessment of buffers, pH, organic co-solvents, surfactants, and sucrose to identify excipients that enhance protein stability. With the knowledge gained from the initial formulation development, formulation development activities for the 150 mg/mL liquid formulation involved assessment of viscosity-reducing excipients, pH, surfactant, and thermal stabilizers to identify excipients that enhance protein stability at higher protein concentrations of between 150 and 200 mg/mL H4H1276S, while maintaining a solution with acceptable viscosity

Throughout formulation development, three primary protein stress conditions (representing extreme handling conditions beyond which the antibody drug product would not be subjected during handling, manufacturing, shipping, storing, and labeling) were employed to develop and optimize the antibody formulations and to evaluate the effects of potential real-world stresses on the stability of the drug product. These stress conditions included:
Agitation (vortexing) of the protein solution at room temperature. Vortexing in glass vials exceeds the agitation that occurs during the handling and manufacturing of the protein.
Incubating the protein solution at elevated temperature (37° C., 40° C. or 45° C.) relative to the proposed DP storage condition (2° C.-8° C.).
Subjecting the protein to multiple freeze thaw cycles. Since the protein will undergo at least one freeze thaw cycle during the manufacture of DP, multiple freeze thaw cycles simulate and exceed the actual stress the protein is expected to experience.

Anti-ANGPTL3 antibodies: Anti-ANGPTL3 antibodies are described in U.S. Pat. No. 9,018,356 B2, incorporated herein in its entirety. The exemplary antibody used in the Examples below is a fully human anti-ANGPTL3 antibody H4H1276S (as disclosed in '356) comprising a heavy chain variable region/light chain variable region HCVR/LCVR amino acid sequence pair comprising SEQ ID NOs: 66/74; and heavy and light chain complementarity determining region CDR sequences comprising SEQ ID NOs: 68/70/72/76/KAS/80; and herein also referred to as “mAb1”.

### Example 2: Exemplary Formulations

In certain embodiments, mAb1 is formulated as an aqueous buffered formulation containing from 5 mg/ml±0.75 mg/ml to 250 mg/ml±45.0 mg/ml mAb1, 10 mM±2 mM histidine, 0.1%±0.05% w/v polysorbate, 50 to 75 mM Arginine-HCl, and 1%±0.02% to 5%±1% w/v proline, at pH 6.0±0.3. Exemplary formulations include: 150 mg/mL H4H1276S, 10 mM histidine, pH 6.0, 70 mM Arginine-HCl, 0.1% (w/v) polysorbate 80, and 3% (w/v) proline.

### Example 3: Methods Used to Assess Formulation Stability

The physical stability of a formulation refers to properties such as color, appearance, pH, turbidity, and protein concentration. The chemical stability refers to the formation of high molecular weight (HMW) species, low molecular weight (LMW) species, charge variants, and other chemical modifications of the protein. The physical and chemical stabilities of antibody drug product (for example, H4H1276S) were assessed using the following assays:
Color and appearance by visual inspection (presence of visible particulates in solution can be detected)
pH
Turbidity measured by increase in optical density (OD) at 405 nm
Subvisible particulate analysis by Micro-Flow Imaging™ (MFI)
Protein concentration by reversed-phase ultra performance liquid chromatography (RP-UPLC), reported as percent protein recovery relative to the starting material
Purity by the following assays:
   Size exclusion ultra performance liquid chromatography (SE-UPLC)
   Reduced and non-reduced microchip capillary electrophoresis sodium dodecyl sulfate (MCE-SDS)
Charge variant analysis:
   Cation exchange UPLC (CEX-UPLC)
   Imaged capillary isoelectric focusing (iCIEF)
Potency by Bioassay:
   The relative potency of each sample is determined by a bioassay and is defined as: (IC50 Reference Sample/IC50 Sample)*100%. The measured potency of storage stability samples must be within 50% and 150% of the measured potency of the reference standard.

For the chemical stability of a formulation, the formation of covalently modified forms (e.g. covalent aggregates, cleavage products, or charge variant forms) and non-covalently modified forms (e.g. non-covalent aggregates) of protein is evaluated. Higher and lower molecular weight degradation products can be separated from native antibody by SE-UPLC and MCE-SDS methods.

### Example 4: Selection of Viscosity-Reducing Agent

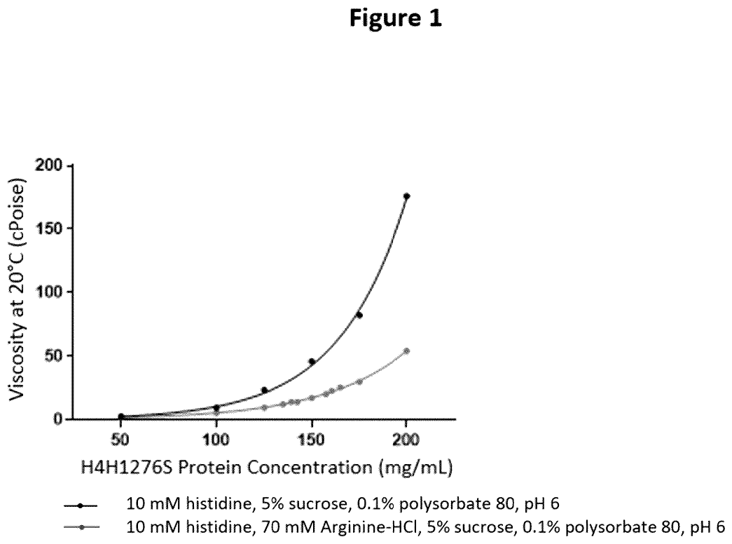

To understand how viscosity is affected by increasing concentrations of H4H1276S, formulations were prepared with different protein concentrations in 10 mM histidine, pH 6.0, 5% sucrose and 0.1% polysorbate 80. This formulation is equivalent to the first-in-human (FIH) formulation used for IV administration in initial clinical studies. The viscosity of each sample was measured at 20° C. and the results are shown in FIG. 1 (top curve). The measured viscosity at 150 mg/mL was greater than 40 centipoise (cP), which is significantly higher that the targeted acceptable viscosity of 20 cP. It was, thus, surmised that use of a viscosity-reducing excipient is required to achieve a formulation with a target protein concentration of 150 mg/mL and acceptable viscosity.

In order to identify a suitable viscosity-reducing excipient, the effect of selected excipients on the H4H1276S formulation viscosity was examined. The excipients included were arginine-HCl, sodium chloride, histidine-HCl, sodium acetate, calcium chloride, magnesium chloride, calcium acetate, and magnesium acetate. The addition of 70 mM arginine-HCl decreased the viscosity at all protein concentrations tested (FIG. 1, bottom curve). Arginine-HCl was found to be the most effective at reducing viscosity compared to the other excipients. Additionally, it had a minimal impact on stability. A summary of excipients tested for viscosity reduction is provided in, below. Arginine-HCl was selected for additional development studies, because it provided adequate reductions in viscosity. Subsequent formulation development studies included 70-75 mM arginine-HCl to determine the optimal pH, surfactant concentration, and thermal stabilizer.

TABLE 1
Summary of Excipients Tested for Viscosity Reduction

| | Concentration | Effect on | Effect on |
|---|---|---|---|
| Excipient | Range | Viscosity | Stability |
| Arginine-HCl | 50-75 mM | Significant | Minimal |
| | | Reduction | |
| Sodium Chloride | 50-75 mM | Significant | Minimal |
| | | Reduction | |
| Histidine-HCl | 40-65 mM | Reduction | Not Tested |
| Sodium Acetate, | 40-60 mM | Significant | Decreased |
| pH 5 | | Reduction | Stability |
| Calcium Chloride | 25 mM | Significant | Minimal |
| | | Reduction | |
| Magnesium Chloride | 25 mM | Significant | Minimal |
| | | Reduction | |
| Calcium Acetate | 25 mM | Significant | Minimal |
| | | Reduction | |
| Magnesium Acetate | 25 mM | Significant | Minimal |
| | | Reduction | |

### Example 5: PH Selection

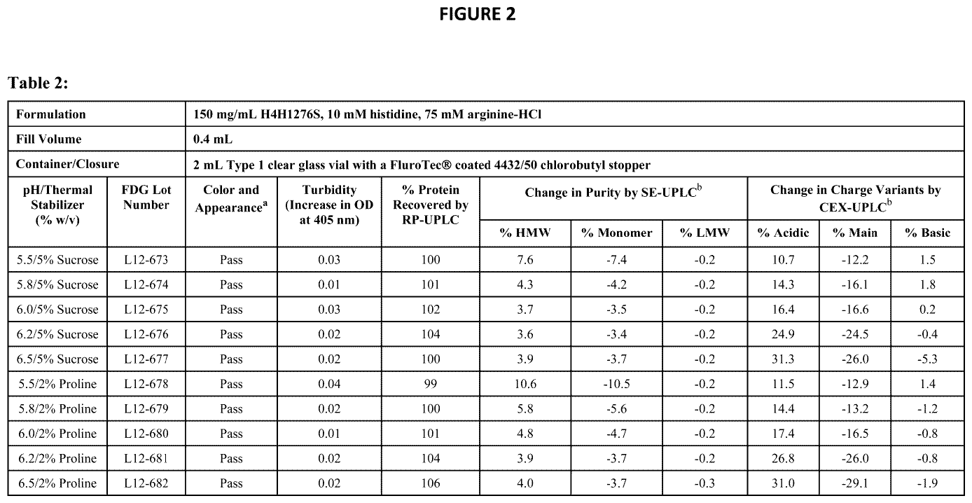

The effect of pH on the thermal stability of H4H1276S was examined in liquid formulations by incubating 150 mg/mL H4H1276S at 45° C. for 28 days at varying pH ranges in 10 mM histidine with either 5% (w/v) sucrose or 2% (w/v) proline. Sucrose and proline were considered as the potential thermal stabilizer and were included, so that the effect of buffer and pH could be studied with a formulation composition that is more representative of the final formulation. Based on results from SE-UPLC and CEX-UPLC analysis (Table 2, as shown in FIG. 2), higher pH minimizes HMW species formation, while lower pH minimizes charge variant formation. Histidine buffer at a pH of 6.0 was selected as the formulation buffer, because it provided the best balance between HMW species formation and charge variant formation.

### Example 6: Surfactant Concentration Optimization

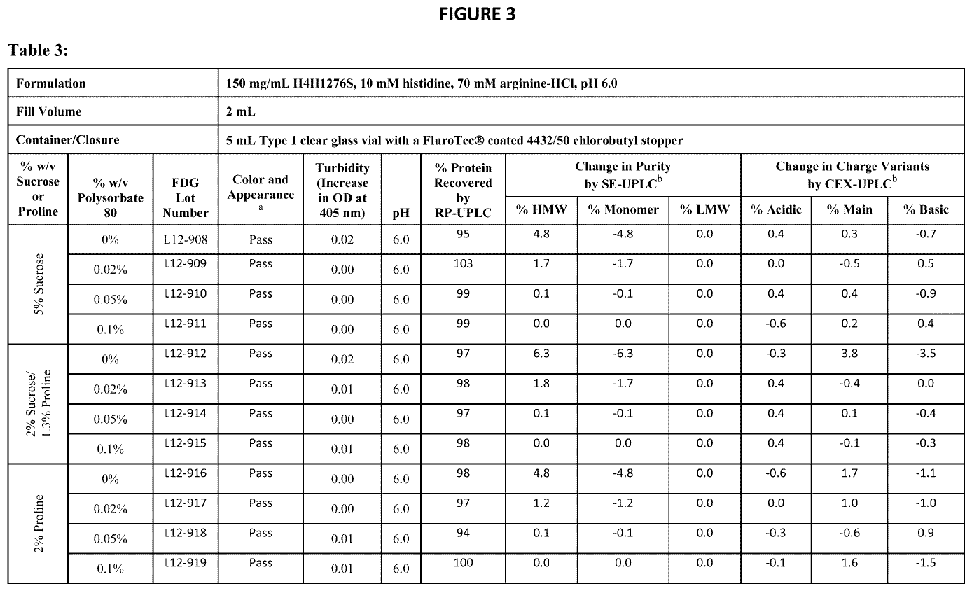

Surfactants are often added to antibody formulations to protect the protein from agitation-induced aggregation. When developing the initial lyophilizable formulation, HMW species formation was observed when 50 mg/mL H4H1276S was agitated. Addition of 0.1% (w/v) polysorbate 80 protected H4H1276S from agitation-induced instability. However, protein concentration, thermal stabilizer content, and the presence of other excipients can affect a protein's susceptibility to agitation stress. Therefore, the minimum amount of polysorbate 80 needed to protect 150 mg/ml H4H1276S from agitation stress was evaluated. Polysorbate 80 concentrations of 0.0%, 0.02%, 0.05%, and 0.1% (% w/v) were examined in the presence of 5% sucrose, 2% sucrose and 1.3% proline, or 2% proline (% w/v). The samples were formulated in 10 mM histidine, pH 6, with 70 mM arginine to be more representative of the final formulation. The results are summarized in Tables 3, 4, and 5 (as shown in FIGS. 3, 4, and 5, respectively).

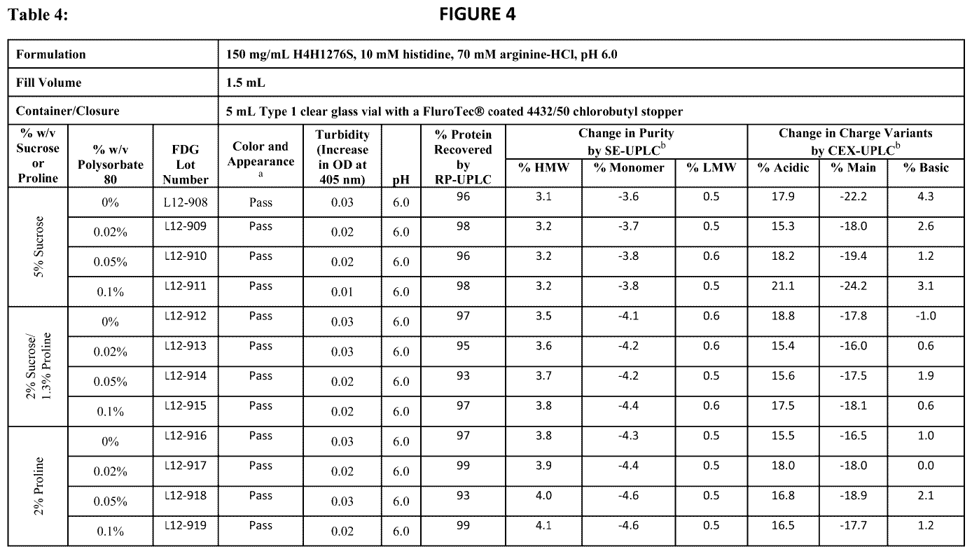

When agitated for 120 minutes (Table 3, as shown in FIG. 3), a significant increase in HMW species (4.8-6.3%) was observed for formulations without surfactant. Addition of 0.02% (w/v) polysorbate 80 was not sufficient to protect H4H1276S against agitation-induced instability, while 0.05% (w/v) polysorbate 80 and above provided adequate stabilization, regardless of thermal stabilizer included in the formulation. The data demonstrate that at least 0.05% (w/v) polysorbate 80 is required to protect H4H1276S from agitation-induced instability, and that with at least 0.05% (w/v) polysorbate 80, the agitation-induced instability is not impacted by choice of thermal stabilizer. The addition of polysorbate 80 does not impact the formation of HMW species of H4H1276S when incubated at 45° C., regardless of thermal stabilizer included in the formation (Table 4, as shown in FIG. 4). The relative change in HMW species from t=0 for the formulations without thermal stabilizer was comparable to those with polysorbate. The increase in HMW species ranged from 3.1-3.2%, 3.5-3.8% and 3.8-4.1% for the sucrose only, sucrose and proline, or proline only formulations, respectively.

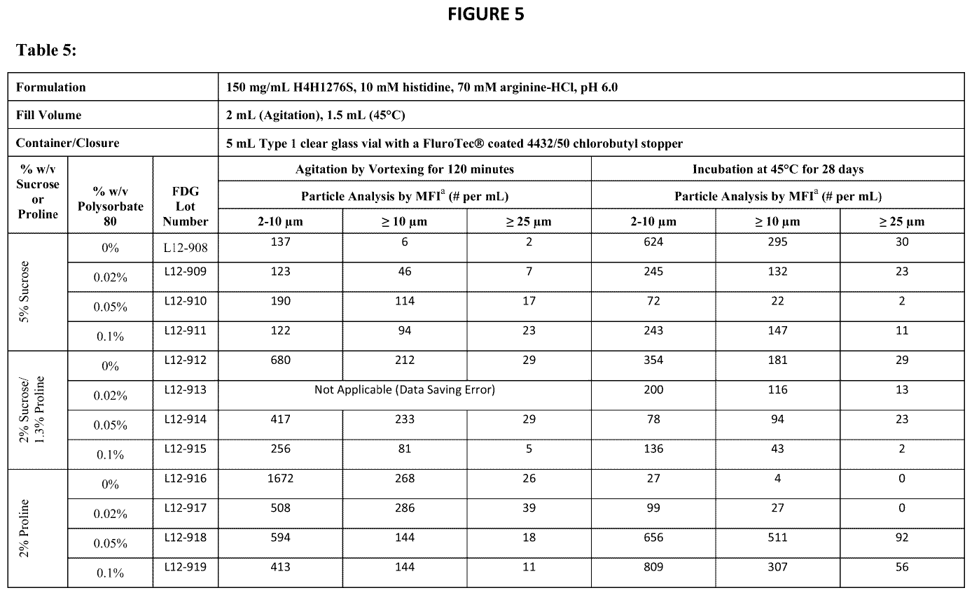

When comparing across thermal stabilizer groups, H4H1276S exhibited modestly improved stability when formulated with sucrose (compared to proline) and incubated under stress conditions. The difference, however, was not considered to be meaningful. The differences in total relative change in charge variant distribution from t=0 for all formulations evaluated is considered to be within assay variability. The impact of agitation and 45° C. incubation on particulate formation was evaluated using MFI, and the results are shown in Table 5 (as shown in FIG. 5). There was no discernable trend in particulate formation, suggesting that particulate formation is not impacted by the concentration of polysorbate 80 or thermal stabilizer included in the formulation. Although stabilization was achieved at 0.05% (w/v) polysorbate concentration, 0.1% (w/v) polysorbate 80 was selected as the surfactant concentration. The higher concentration of polysorbate ensures the robustness of the formulation, has equivalent stability as 0.05% (w/v) polysorbate formulation, and provides additional stabilization upon dilution into an IV bag.

### Example 7: Effect of Thermal Stabilizer

Stabilizers can be added to antibody formulations to increase the stability of the protein in liquid formulations and during frozen storage. Sucrose was included as a thermal stabilizer in previous formulations. However, sucrose can also increase the viscosity of the solution. Therefore, proline was evaluated as a thermal stabilizer, in the hopes that it could influence the solubility and colloidal stability of protein to improve the storage stability of monoclonal antibody product formulation, without increasing the viscosity of the final formulation (Table 6, below).

TABLE 6
Viscosity at 20° C. for H4H1276S Formulations
with Different Concentrations of Sucrose and Proline

| Formulation Composition | | | | | | | |
|---|---|---|---|---|---|---|---|
| H4H1276S | Histidine | | Polysorbate | Arginine-HCl | Sucrose | Proline | Viscosity at |
| (mg/mL) | (mM) | pH | 80 (% w/v) | (mM) | (% w/v) | (% w/v) | 20° C. |
| 150 | 10 | 6 | 0.1 | 70 | 5 | 0 | 15.3 |
| 150 | 10 | 6 | 0.1 | 70 | 3 | 1 | 14.8 |
| 150 | 10 | 6 | 0.1 | 70 | 2 | 1.5 | 14.0 |
| 150 | 10 | 6 | 0.1 | 70 | 0 | 2 | 13.1 |

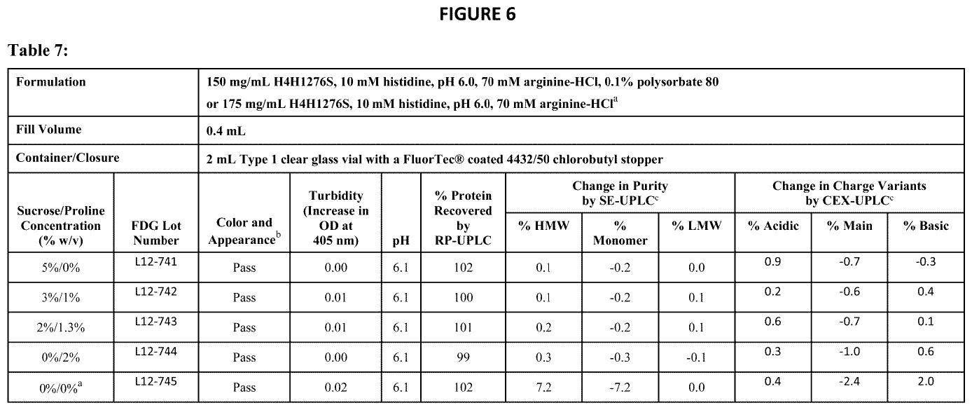

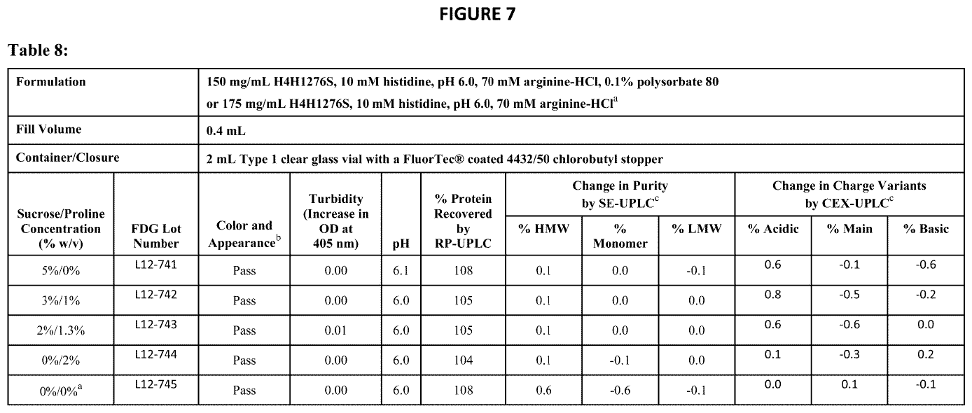

To support selection of the components for the bulk formulated drug substance composition and to assess the need for the presence of a thermal stabilizer in the formulation, the stability of 175 mg/mL H4H1276S in 10 mM histidine, pH 6.0 and 70 mM arginine-HCl (no sucrose or proline) was evaluated by assessing frozen storage stability at −20° C. and freeze/thaw stability (frozen at −30° C. and thawed at room temperature) (Tables 7 and 8 (shown in FIGS. 6 and 7, respectively)). Although −30° C. is the intended long-term storage condition for formulated drug substance, frozen storage stability at −20° C. was evaluated as an accelerated frozen storage condition for development. A 7.2% increase in HMW species was observed following incubation at −20° C. for 9 months (Table 7, shown in FIG. 6). While a small increase in HMW species was observed for this formulation following 8 freeze/thaw cycles (Table 8, shown in FIG. 7), the −20° C. frozen storage stability data indicate the need for a thermal stabilizer to support long-term bulk drug substance frozen storage.

To identify and optimize the concentration of thermal stabilizer(s) in the liquid formulation, 150 mg/mL H4H1276S was formulated in 10 mM histidine, pH 6, 70 mM arginine-HCl, and 0.1% (w/v) polysorbate 80 and incubated at 45° C. with different concentrations of sucrose and proline to assess protein stability. The formulations were also incubated at −20° C. and subjected to freeze/thaw cycles (−30° C. freeze; room temperature thaw) to compare frozen storage stability, which is needed to support storage of the bulk formulated drug substance. Polysorbate 80 was included in these formulations to be more representative of the final product formulation. Viscosity at 20° C. was measured for all samples at t=0 and is summarized in Table 6, above. Table 6 illustrates that replacement of sucrose with proline decreases the viscosity in a concentration-dependent manner, indicating that formulations with proline may be advantageous for the final product, if there is no negative impact on stability.

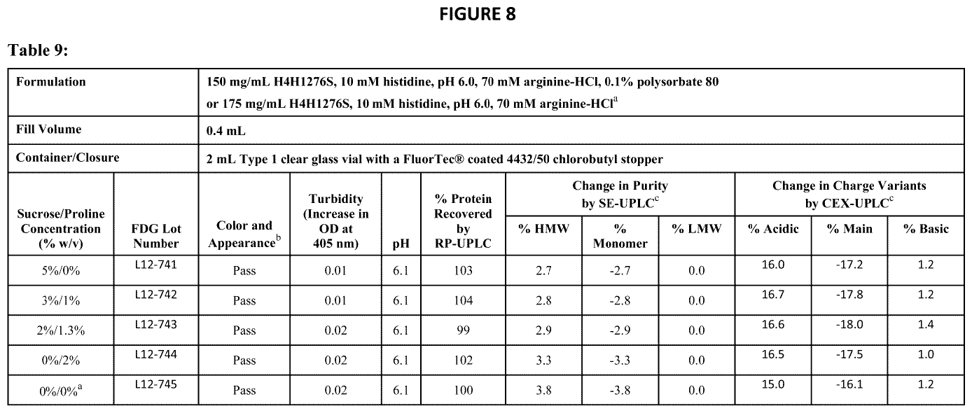

H4H1276S exhibited modestly improved stability when formulated with sucrose compared to proline and incubated at 45° C. for 21 days (Table 9, shown in FIG. 8). The total relative change from t=0 for the formation of HMW species for the 5% sucrose formulation was 2.7%, whereas the total relative change from t=0 in HMW species for the 2% proline formulation was 3.3%. This difference is not considered to be meaningful. The differences in total relative change from t=0 for charge variants are considered to be within assay variability across the different formulations that were evaluated.

No meaningful change in any quality attribute was observed for all formulations containing sucrose and/or proline when incubated at −20° C. or subjected to 8 freeze/thaw cycles (−30° C. freeze; room temperature thaw) (Tables 7 and 8, shown in FIGS. 6 and 7). In summary, the proline-containing formulation exhibited a lower viscosity and comparable stability relative to the sucrose-containing formulation when stored under accelerated or stress storage conditions. Two lead formulations were selected based on these results: 1) 150 mg/mL H4H1276S, 10 mM histidine, pH 6.0, 70 mM arginine-HCl, 0.1% (w/v) polysorbate 80, and 5% (w/v) sucrose; and 2) 150 mg/mL H4H1276S, 10 mM histidine, pH 6.0, 70 mM arginine-HCl, 0.1% (w/v) polysorbate 80, and 3% (w/v) proline. The proline concentration was increased slightly to ensure adequate protein stability under representative long-term storage conditions.

### Example 8: Formulation Selection

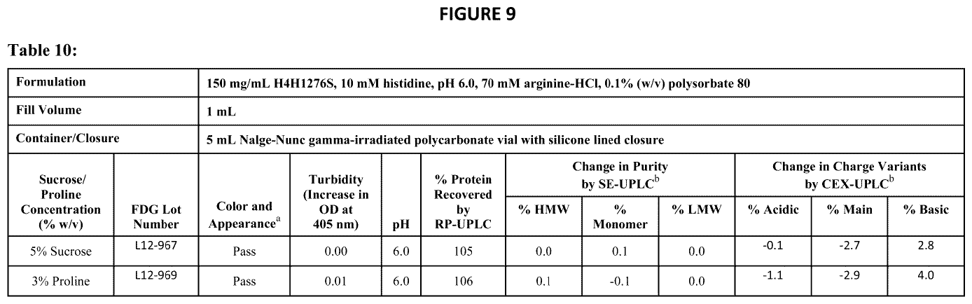

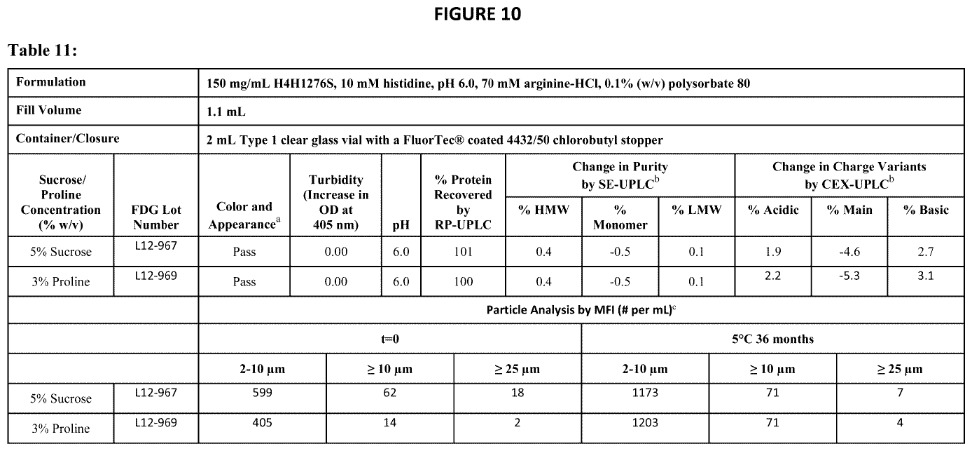

Long-term stability was examined to compare stability profiles for the two lead formulations (see Example 7, above). Stability data at −30° C. were collected to evaluate the long-term storage of bulk formulated drug substance (Table 10, shown in FIG. 9). Stability data at 5° C. were collected to evaluate the long-term storage of drug product (Table 11, shown in FIG. 10). The data indicate that equivalent stability profiles are obtained with both lead formulations.

The relationship between viscosity, protein concentration, and temperature was used to facilitate the selection of a formulation that would be deliverable at a range of protein concentrations and temperatures. The impact of protein concentration on the viscosity of the final formulation, and how excipients affect that relationship, were considered. Likewise, the temperature was considered, with a view to a) withdrawal and/or administration of the drug (about room temperature); b) bulk manufacturing process steps (typically about 15-25° C.); and c) storage.

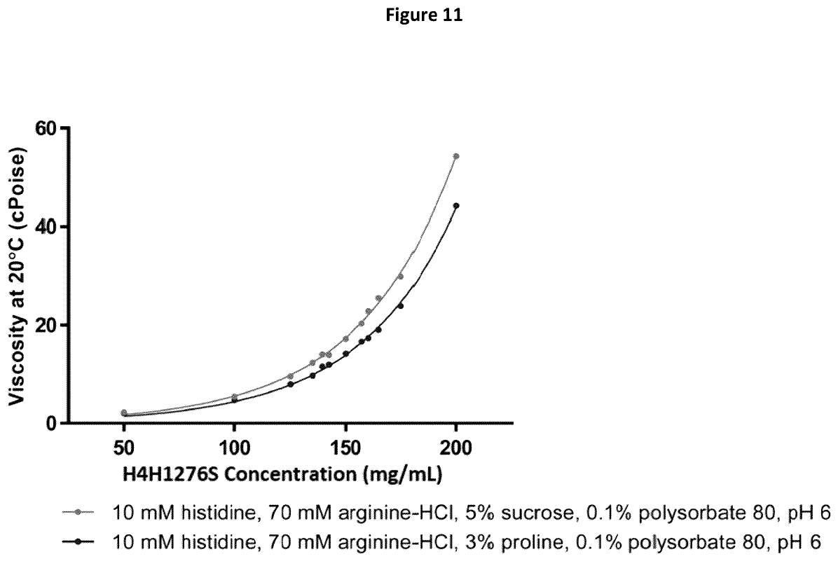

To guide selection of the final formulation and better characterize the impact of thermal stabilizers on viscosity, formulations were prepared with different H4H1276S concentrations in the two lead formulations (10 mM histidine, pH 6.0, 70 mM arginine-HCl, 0.1% (w/v) polysorbate 80, and 5% (w/v) sucrose; and 10 mM histidine, pH 6, 70 mM arginine-HCl, 0.1% (w/v) polysorbate 80, and 3% (w/v) proline). The viscosity was measured at temperatures ranging from 5° C. to 35° C. FIG. 11 illustrates the viscosity versus H4H1276S concentration relationship for both formulations at 20° C. The proline-containing formulation (bottom curve of FIG. 11) had consistently lower viscosities at multiple H4H1276S concentrations when compared to the sucrose-containing formulation (top curve of FIG. 11).

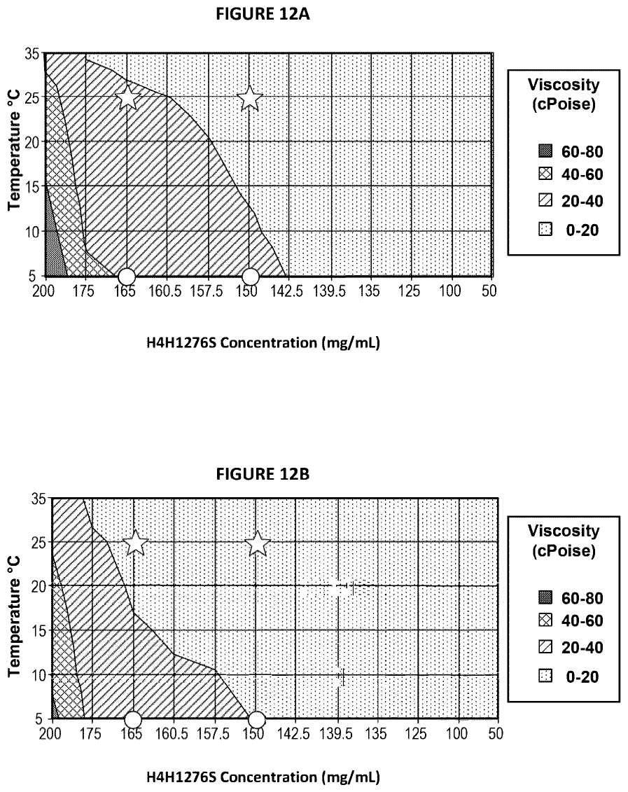

This trend was even more apparent at lower temperatures (FIGS. 12A and 12B). The data indicate that the formulation containing 3% (w/v) proline (FIG. 12B) offers a wider operational space of viscosity values that are considered to be acceptable. Because the stabilities of the two lead formulations are equivalent, and the proline-containing formulation has an advantageous viscosity profile, the formulation containing 3% (w/v) proline was selected for the final drug product formulation.

The final H4H1276S liquid drug product formulation contains 150 mg/mL H4H1276S, 10 mM histidine, 70 mM arginine-HCl, 3% (w/v) proline, and 0.1% (w/v) polysorbate 80 at pH 6.0. The main degradation pathways identified during development were high molecular weight species and charge variants. The final osmolality of the formulation is approximately 480 mOsm/kg, and the viscosity is approximately 15 cPoise (20° C.), which is suitable for clinical use.

A summary of the H4H1276S formulations developed for preclinical (GLP Toxicology) and clinical use are outlined in Table 12, below.

TABLE 12
Composition of H4H1276S Formulations used in GLP toxicology studies and
H4H1276S clinical DP Formulations

| | Frozen | | | | | | | |
|---|---|---|---|---|---|---|---|---|
| | Liquid | | Lyophilized DP (Clinical Use) | | | | | |
| | (GLP | | Reconstituted | | Reconstituted | | Liquid DP | |
| Formulation | Toxicology | | for IV | | for SC | | (Clinical Use) | |
| Component | Use) | | Administration | | Administration | | for IV or SC | |
| H4H1276S | 50 | mg/mL | 50 | mg/mL | 100 | mg/mL | 150 | mg/mL |
| Histidine | 10 | mM | 10 | mM | 20 | mM | 10 | mM |
| Arginine-HCl | Not applicable | | Not applicable | | Not applicable | | 70 | mM |
| Sucrose | 5% | (w/v) | 5% | (w/v) | 10% | (w/v) | Not applicable | |
| Proline | Not applicable | | Not applicable | | Not applicable | | 3% | (w/v) |
| Polysorbate 80 | 0.1% | (w/v) | 0.1% | (w/v) | 0.2% | (w/v) | 0.1% | (w/v) |

| | | | | |
|---|---|---|---|---|
| pH | 6 | 6 | 6 | 6 |

### Example 9: Summary of Stability Studies for H4H1276S Drug Product

Studies were carried out to evaluate the storage and accelerated stability of H4H1276S 150 mg/mL drug product (DP) (data not shown). Stability was assessed in terms of color and appearance, turbidity (increase in OD at 405 nm), pH, particulate matter by MFI, % total protein recovered by RP-UPLC (reversed phase ultra performance liquid chromatography), % purity by non-reduced and reduced MCE-SDS (microchip capillary electrophoresis-sodium dodecyl sulfate), % purity by SE-UPLC (size exclusion ultra performance liquid chromatography), charge variant analysis by CEX-UPLC (cation exchange ultra performance liquid chromatography), charge variant analysis by iCIEF (imaged capillary isoelectric focusing), and % relative potency by bioassay.

The DP used for the storage and accelerated stability study was manufactured by incubating 5.0 mL of formulated drug substance (FDS) into a 20 mL Type 1 clear glass vial. H4H1276S DP was physically and chemically stable when stored at 5° C. for at least 12 months. No appreciable change in the physical or chemical stability was detected in any of the monitored attributes.

Incubation followed at either 25° C./60% Relative Humidity (accelerated stability) or 45° C. (stress stability). These accelerated and stress conditions were selected to elucidate the degradation pathways for H4H1276S. As to the accelerated stability studies, after 3 months at 25° C./60% RH, appreciable formation of HMW species and charge variants were detected. No appreciable formation of HMW species or charge variant formation was observed after incubation for 1 month at 25° C./60% RH, indicating that H4H1276S DP can be exposed to room temperature for short periods of time. H4H1276S maintained potency, as determined by bioassay analysis, after incubation under the accelerated condition.

Incubation at 45° C. resulted in significant formation of HMW species and charge variants, in as little as 7 days, demonstrating that the increase in HMW species and the formation of charge variants are the main degradation pathways for H4H1276S DP. H4H1276S DP was found to be physically and chemically stable when agitated (vortexed at ambient temperature) for 120 minutes or when subjected to 8 freeze/thaw cycles (freezing at −30° C. and thawing at room temperature). No appreciable change in the physical or chemical stability was detected in any of the monitored attributes.

The results from the DP storage accelerated and stress stability studies indicate that H4H1276S 150 mg/mL DP will be stable during manufacture and storage. Furthermore, H4H1276S formulation can withstand short exposures to room temperature without compromising physical or chemical stability. H4H1276S 150 mg/mL DP will preferably be stored at 2° C. to 8° C., with exposure to temperatures greater than 2° C. to 8° C. limited.

### Example 10: Development of an Amino Acid Based High Concentration Liquid Drug Product for H4H1276S, from Excipient Screening to Characterization of Rheological Properties

H4H1276S was lyophilized and then reconstituted to higher protein concentrations with solutions containing the desired excipients. The pre-lyophilized formulation contained 4 mL of 87.5 mg/mL H4H1276S, with 10 mM Histidine, pH 6 and 2.5% (w/v) Sucrose. The lyophilized cake contained 350 mg of solid H4H1276S. The lyophilized cake was reconstituted with 2-2.4 mL solution to yield a final H4H1276S concentration of 160-175 mg/ml (nominal). The reconstitution solution was adjusted such that the final sample contained 20 mM Histidine, pH 6, with 5% (w/v) sucrose and the test excipient. To adjust the pH from 6 to 5, acetate buffer was added to the reconstitution solution, such that the final acetate concentration was 40 mM.

To investigate the accelerated and frozen storage stability of H4H1276S formulations containing viscosity-reducing excipients, the viscosity of the test formulations was measured at 20° C. using the Rheosense Viscometer. The test formulations were incubated in 2 mL glass vials at the following conditions: (i) 45° C. for 0, 7, 14 and 21 days; (ii)−20° C. for 0, 1, 2, 3, 6, and 9 months; and (iii) 5° C., −30° C. and −80° C. for 0 and 3 months. The resulting material was assayed for aggregate content by SEC and for charge variant formation by CEX.

To understand the relationship between protein concentration, temperature, and viscosity for H4H1276S formulations with reduced viscosity, 200 mg/mL H4H1276S formulations containing the following combinations of excipients were prepared. All formulations contained 10 mM histidine at pH 6 with 0.1% (w/v) polysorbate 80:
5% (w/v) Sucrose
5% (w/v) Sucrose, 70 mM L-Arg-HCl
3% (w/v) Sucrose, 1.3% (w/v) Proline, 70 mM L-Arg-HCl
3% (w/v) Proline
3% (w/v) Proline, 70 mM L-Arg-HCl.
   The 200 mg/mL formulation was diluted with the respective formulation buffer to protein concentrations ranging from 50-200 mg/mL. Viscosity measurements of each formulation were performed on the Rheosense Viscometer at temperatures ranging from 5-35° C. Finally, data analysis was performed in GraphPad Prism and MiniTab.

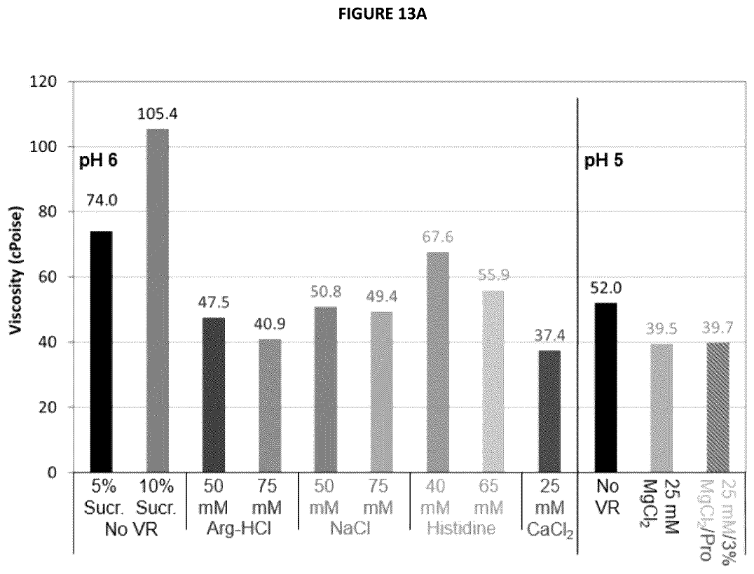

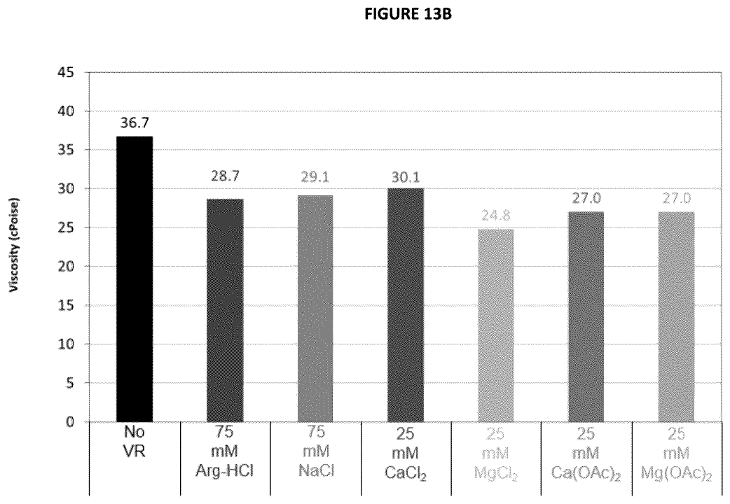

The results of the screening for viscosity-reducing excipients are shown in FIGS. 13A and 13B. For FIG. 13A, upon reconstitution, the base formulation contained 175 mg/mL H4H1276S, 20 mM Histidine, pH 6, 0.1% (w/v) polysorbate 80, 5% (w/v) sucrose. To achieve a pH 5 solution, acetate was added at a final concentration of 40 mM. An equivalent formulation containing 40 mM acetate, pH 6.0, had a viscosity of 64.1 cPoise, indicating that both addition of acetate and pH adjustment to 5.0 reduce the viscosity of H4H1276S formulations. For FIG. 13B, upon reconstitution, the base formulation contained 165 mg/mL H4H1276S, 20 mM Histidine/40 mM Acetate, pH 5.0, 0.1% (w/v) polysorbate 80 and 5% (w/v) sucrose.

The addition of monovalent and divalent salts (L-Arg-HCl, NaCl, CaCl₂, MgCl₂, Ca(OAc)₂, Mg(OAc)₂) reduced the viscosity of H4H1276S formulations. The viscosity of H4H1276S formulations was also reduced by adjusting the pH from 6 to 5. Finally, sucrose increased the viscosity, while L-proline did not affect the viscosity, of H4H1276S formulations.

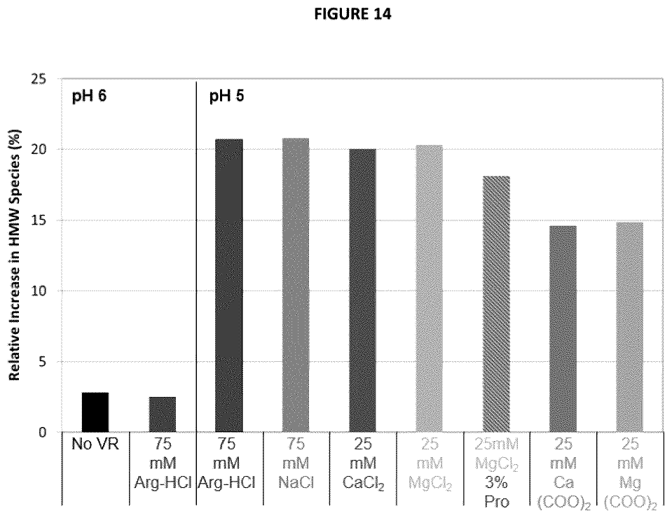

The formation of high molecular weight (HMW) species following 21 days incubation at 45° C. is shown in FIG. 14. Select H4H1276S formulations containing various VR (viscosity-reducing) excipients were incubated at 45° C. and analyzed by SEC for aggregate content. The relative increase in HMW species from the t=0 sample is plotted as a function of formulation. Under accelerated conditions (45° C. for 21 days), the following was observed:
L-Arg-HCl had little impact on the formation of HMW species;
Reducing the pH of H4H1276S formulations led to increased formation of HMW species;
The relative increase in HMW species was similar for the CaCl2- and MgCl2-containing formulations compared to the L-Arg-HCl- and NaCl-containing formulations;
L-proline acted as a thermal stabilizer and reduced the formation of HMW species; and
Compared to the L-Arg-HCl formulation, Ca(OAc)2- and Mg(OAc)2-containing formulations had reduced HMW species formation.
   Thus, L-Arg-HCl or Mg(OAc)₂ (substantial viscosity reduction) in pH 6 buffer (better accelerated stability) were chosen as the lead viscosity reducers for further development.

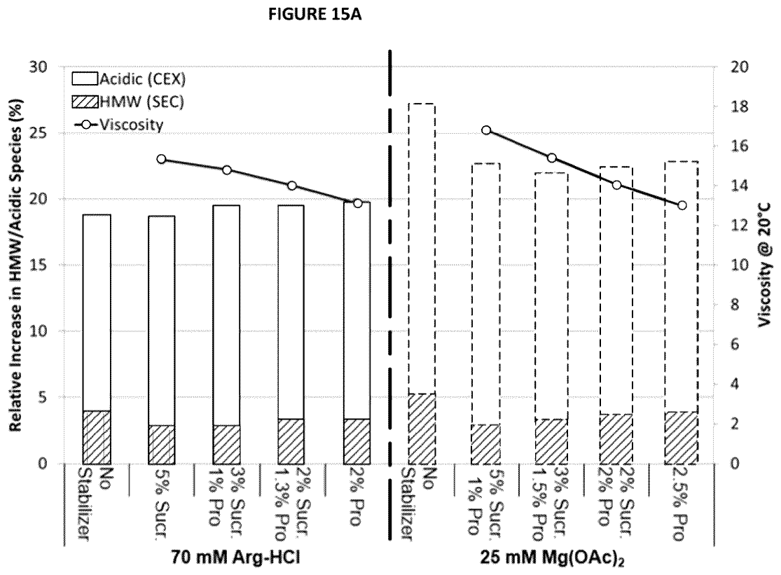

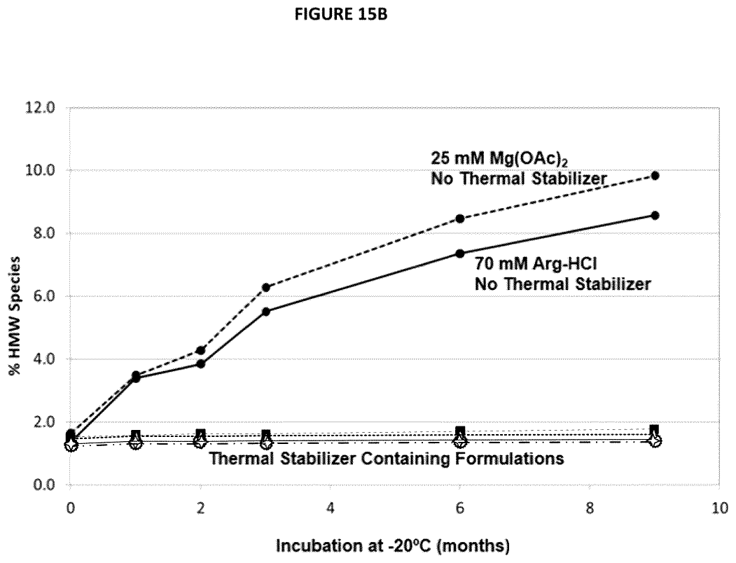

The stability of H4H1276S formulations containing viscosity-reducing excipients is summarized in FIGS. 15A and 15B. 150 mg/mL H4H1276S formulations containing either 70 mM L-Arg-HCl or 25 mM Mg(OAc)₂ were formulated with various concentrations of sucrose and/or L-proline. The concentrations were adjusted to target an osmolality of approximately 300 mM to maintain isotonicity. Formulations containing only L-Arg-HCl or Mg(OAc)₂ were prepared at 175 mg/mL H4H1276S. For FIG. 15A, depicting the degradation of H4H1276S following 21 days incubation at 45° C., samples were incubated at 45° C. and analyzed by SEC for aggregate content and CEX for acidic charge variant formation. The relative increase in HMW or acidic species from the t=0 sample is plotted as a function of formulation. For FIG. 15B, depicting the frozen storage stability of H4H1276S, samples were incubated at −20° C. for 9 months and analyzed by SEC for aggregate content. The percentage of HMW species is plotted as a function of time. Test formulations were also incubated at −80° C., −30° C. and 5° C. for 3 months (data not shown). No change in HMW species was observed at −80° C. for any of the formulations. At 5° C. or −30° C., the formulations without thermal stabilizer had increased HMW species content.

Mg(OAc)₂ formulations were slightly less stable at accelerated storage conditions than those containing L-Arg-HCl, with increased formation of both HMW species and acidic species. Replacement of sucrose with L-proline decreased the viscosity in a concentration-dependent manner. The addition of sucrose, L-proline, or a combination of both excipients sufficiently protected H4H1276S from HMW species formation at −20° C. Thus, 70 mM L-Arg-HCL was selected as the lead viscosity reducer for further development. The data presented support the use of Mg(OAc)₂ as a back-up excipient.

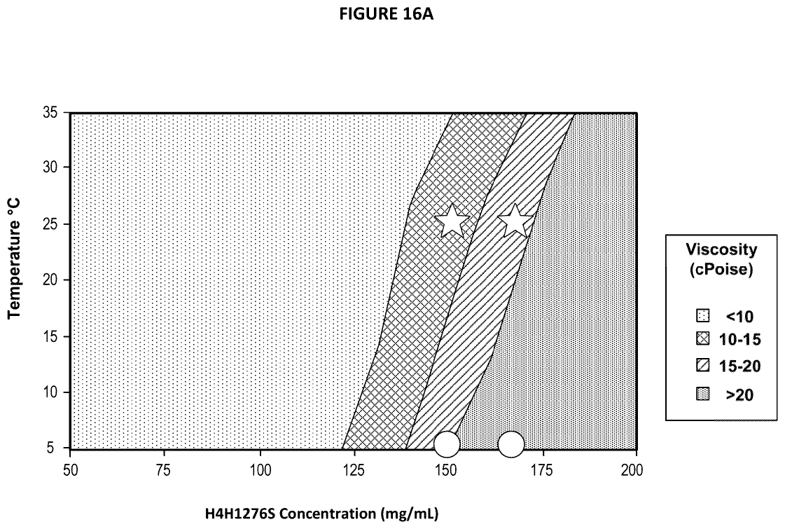

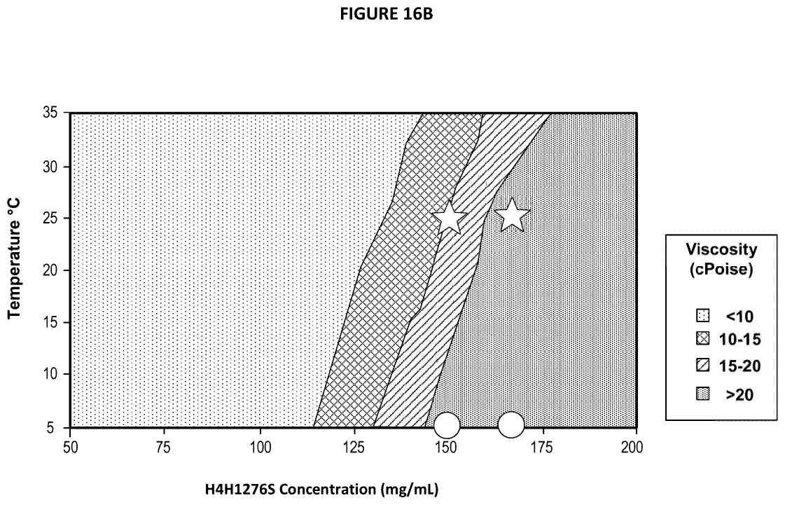

The relationship between the protein concentration, temperature, and viscosity is shown in FIGS. 16A and 16B. These figures show contour plots of viscosity versus protein concentration and temperature. The contour plots were generated in Minitab. The shapes at points corresponding to 150 mg/mL and 165 mg/mL (or 150 mg/mL+10%) H4H1276S are at the recommended storage temperature of 5° C. (circle) or recommended administration temperature of 25° C. (star).

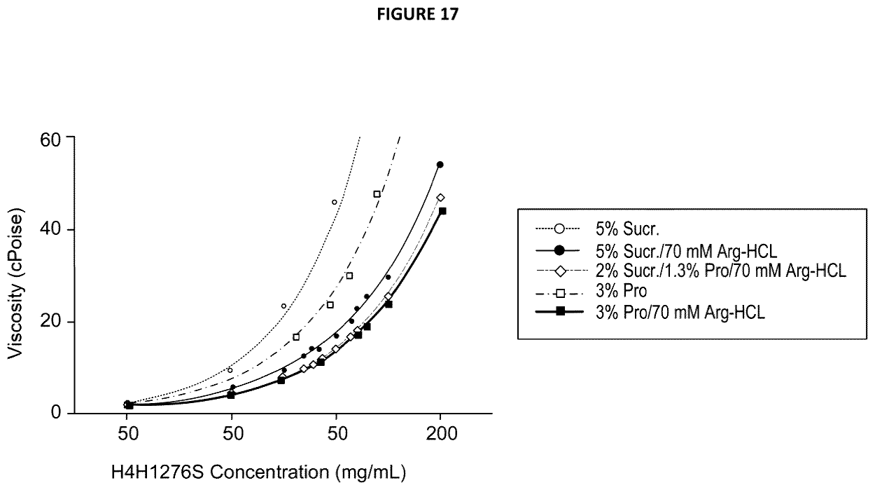

The relationship between viscosity and protein concentration at 20° C. is shown in FIG. 17. Viscosity was plotted as a function of protein concentration, with the data fit to an exponential curve using GraphPad Prism. The equation(s) can be used to predict viscosity based on known concentration, which is useful for defining manufacturing specifications in the final product and informing process development. The equation for the formulation containing 3% L-proline and 70 mM L-Arg-HCl is: viscosity=0.444 e^{0.023[H4H1276S]}.

The above-mentioned contour plots and graph support the following observations: Compared with the sucrose-containing formulations, the L-proline-containing formulations had consistently lower viscosities at multiple H4H1276S concentrations (and temperatures; data not shown) with or without 70 mM L-Arg-HCl.

The viscosity versus protein concentration curve (at 20° C.) for the formulation containing a combination of 5% sucrose and 3% L-proline was very similar to the 3% proline formulation. Differentiation between these two formulations was, in fact, more apparent at lower temperatures and higher protein concentrations. The contour plots for these formulations had subtle, but distinct differences (data not shown)

When considering both temperature and manufacturing specifications, the L-proline formulation offered a wider operation space of viscosity values that are considered acceptable for SC administration (FIG. 16A).

The amino acid-based formulation containing 150 mg/mL H4H1276S, 10 mM Histidine, pH 6 with 70 mM L-Arg-HCl, and 3% proline was selected for H4H1276S DP development.

The formulation containing a combination of both sucrose and L-proline was eliminated for further development, because it offered no advantage in terms of frozen storage stability (see above).

Thus, a thorough understanding of the relationship between the viscosity, protein concentration, and temperature have been found herein to inform decision-making and facilitate selection of a formulation that is stable upon storage and deliverable in a pre-filled syringe or auto injector format. The viscosity should ideally be considered at a range of protein concentrations, and a target concentration selected located on the lower end of the viscosity versus protein concentration curve, in order to account for manufacturing specifications of the DP and process related manufacturing steps (i.e., bulk drug substance).

### Example 11: Compatibility with Intravenous Delivery Device

For delivery in a clinical setting, 150 mg/mL H4H1276S DP can be diluted in an intravenous (IV) bag containing normal saline for IV administration, at clinical doses of 5 mg/kg and 15 mg/kg. The in-use stability of H4H1276S to support IV administration of the clinical doses was assessed. Two admixture concentrations, 0.5 mg/mL H4H1276S and 20 mg/mL H4H1276S, were examined in an effort to bracket the low and high concentrations of admixtures that could be administered in a clinical setting.

In order to evaluate delivery of the admixture from IV bags using an IV pump and infusion set containing an inline filter, normal saline-containing IV bags made of polyvinyl chloride (PVC) with Di-(2-ethylhexyl) phthalate (DEHP) and two types of commonly used infusion pumps (peristaltic and fluid displacement) were tested. Several infusion sets containing the basic materials (PVC with DEHP, PVC with TO™ and polyethylene) and a 0.2 μm polyethersulfone inline filter were also evaluated.

### Assays

The compatibility of the H4H1276S admixture with materials used in IV dosing device was assessed using the following assays:
Color and appearance by visual inspection
pH
Turbidity measured by increase in Optical Density (OD) at 405 nm
Subvisible particulate analysis on admixture by light obscuration (HIAC)
Protein concentration by reversed-phase high performance liquid chromatography (RP-UPLC)
Purity by SE-UPLC
Potency, by bioassay: the relative potency of each sample is determined using the bioassay and is defined as: (IC50 Reference Sample/IC50 Sample)×100%. The measured potency of storage stability samples must be within 50-150% of the measured potency of the reference standard.

### Study Procedure

100 mL normal saline IV bags containing H4H1276S DP were subjected to various stress conditions to assess whether H4H1276S is stable in the admixture and when delivered intravenously. The IV bags containing the admixture were initially held for 24 hours at 5° C.; the bags were then incubated for at least 8 hours at 25° C. After these incubations were complete, each of the evaluated infusion sets was connected to an IV bag, primed with the admixture, and held for 1 hour at ambient room temperature. Each admixture was then pumped through the respective infusion set at rates of either 25 mL/hr or 500 mL/hr. In the clinic, the doses could be administrated using DP diluted in either 100 mL or 250 mL IV bags. For the compatibility study, 100 mL IV bags were used to test each dose.

### Study Results

0.5 mg/mL and 20 mg/mL H4H1276S, diluted in saline, were physically and chemically stable under all conditions tested, including the following conditions: i) 5° C. for 24 hours in an IV bag, ii) 25° C. for 8 hours in an IV bag, and iii) ambient temperature for one hour in all infusion sets tested.

In addition, H4H1276S admixtures were stable when pumped through each of the evaluated infusion sets utilizing the various infusion pumps at rates of 25 mL/hr and 500 mL/hr. Precipitate was not detected by visual inspection or turbidity measurement. The pH of the solutions were stable, and no appreciable decreases in protein concentration were observed. No appreciable changes in the relative percentage of high or low molecular weight species were observed in this compatibility study, as determined by size exclusion ultra-performance liquid chromatography (SE-UPLC). No meaningful changes in levels of subvisible particulates, compared to t=0, were observed after samples were pumped through the infusion sets at 500 mL/hr, as determined by HIAC analysis. Finally, all samples tested maintained potency, as determined by Bioassay.

The data support the following dose preparation and administration in the clinic:
Normal saline IV bags made of PVC with DEHP are compatible with H4H1276S for IV administration.
H4H1276S can be diluted to concentrations as low as 0.5 mg/mL in PVC IV bags containing normal saline for IV administration;
H4H1276S can be diluted as high as 20.0 mg/mL in PVC IV bags containing normal saline for IV administration;
H4H1276S admixture in normal saline was stable after incubation in a PVC IV bag for up to 24 hours at 5° C. and 8 hours at 25° C. The diluted H4H1276S admixture will be administered within four hours of preparation;
The H4H1276S admixture in normal saline can be administered using a standard infusion pump;
The H4H1276S admixture can be administered with an infusion set composed of either PVC containing DEHP, PVC containing TO™, or polyethylene;
The H4H1276S admixture is compatible with the use of an inline 0.2 μm polyethersulfone filter;
The H4H1276S admixture can be administered at a flow rate ranging from 25 to 500 mL/hr.

### Example 12: Further Stability Studies for H4H1276S Drug Product

The H4H1276S formulation was subjected to extended storage at −20° C. and 5° C. over periods up to 36 months and analyzed for stability.

First, the formulation was 150 mg/mL H4H1276S, 10 mM histidine, pH 6.0, 70 mM arginine-HCl, 3% (w/v) proline, and 0.1% (w/v) polysorbate 80. 2.0 mL fill volume in a 5 mL Nalge-Nunc gamma-irradiated polycarbonate vial with silicone-lined closure.

TABLE 13
Research Stability of H4H1276S Formulated Drug Substance Stored at −20° C.

| | | Length of Storage at −20° C. (months) | | | | | | | | |
|---|---|---|---|---|---|---|---|---|---|---|
| Assay | | 0 | 1 | 3 | 6 | 9 | 12 | 18 | 24 | 36 |
| Color and Appearance | | Pass | Pass | Pass | Pass | Pass | Pass | Pass | Pass | Pass |
| Turbidity (Increase in | | 0.00 | 0.00 | 0.00 | 0.00 | 0.00 | 0.00 | 0.00 | 0.01 | 0.00 |
| OD at 405 nm) | | | | | | | | | | |
| pH | | 6.0 | 6.0 | 6.0 | 6.0 | 6.1 | 6.0 | 6.0 | 6.1 | 6.0 |
| % Total Protein | | 100 | 103 | 102 | 103 | 102 | 105 | 102 | 106 | 107 |
| Recovered by RP-UPLC | | | | | | | | | | |
| Purity by | Non-reduced; | 95.1 | NR | NR | 94.5 | NR | 94.9 | NR | NR | 96.5 |
| MCE-SDS | % main peak | | | | | | | | | |
| | Reduced; | 97.8 | NR | NR | 91.8 | NR | 99.4 | NR | NR | 99.1 |
| | % heavy + | | | | | | | | | |
| | light chain | | | | | | | | | |
| Purity | % HMW | 1.2 | 1.2 | 1.3 | 1.3 | 1.3 | 1.3 | 1.3 | 1.3 | 1.4 |
| by SE-UPLC | % Monomer | 98.8 | 98.8 | 98.7 | 98.7 | 98.6 | 98.7 | 98.6 | 98.6 | 98.6 |
| | % LMW | 0.0 | 0.0 | 0.0 | 0.0 | 0.0 | 0.0 | 0.0 | 0.0 | 0.1 |
| Charge Variant | % Acidic | 28.5 | 29.1 | 28.8 | 29.8 | 30.1 | 29.9 | 29.1 | 29.5 | 29.1 |
| Analysis | % Main | 47.3 | 46.8 | 48.1 | 46.5 | 48.4 | 49.8 | 48.9 | 46.8 | 46.5 |
| by CEX-UPLC | % Basic | 24.2 | 24.2 | 23.1 | 23.7 | 21.6 | 20.3 | 22.1 | 23.7 | 24.5 |
| Charge Variant | % Acidic | 37.9 | NR | NR | 38.3 | NR | 38.4 | NR | 37.0 | 36.1 |
| Analysis | % Main | 50.2 | NR | NR | 49.8 | NR | 49.1 | NR | 50.0 | 52.0 |
| by iCIEF | % Basic | 11.9 | NR | NR | 11.9 | NR | 12.5 | NR | 13.0 | 11.9 |

Next, the formulation was 150 mg/mL H4H1276S drug product, 10 mM L-histidine, pH 6.0, 70 mM arginine-HCl, 3% (w/v) L-proline, and 0.1% (w/v) polysorbate 80. 5.0 mL fill volume in a 20 mL Type 1 clear glass vials with a 20 mm FluroTec® coated West S2-451 4432/50 GRY B2-40 stoppers.

TABLE 14
Research Stability of H4H1276S Drug Product stored at 5° C.

| | | | Length of Storage at 5° C. (months) | | | | | | | |
|---|---|---|---|---|---|---|---|---|---|---|
| Assay | | | 0 | 1 | 3 | 6 | 9 | 12 | 18 | 24 |
| Color and Appearance | | | Pass | Pass | Pass | Pass | Pass | Pass | Pass | Pass |
| Turbidity (Increase in OD at 405 nm) | | | 0.00 | 0.01 | 0.00 | 0.00 | 0.00 | 0.00 | 0.00 | 0.00 |
| pH | | | 6.0 | 6.0 | 6.1 | 6.1 | 6.1 | 6.0 | 6.0 | 6.0 |
| Particulate | 2-10 | μm | 131 | NR | NR | 1406 | NR | 1104 | NR | 888 |
| Analysis | ≥10 | μm | 0 | NR | NR | 19 | NR | 15 | NR | 6 |
| by MFI | ≥25 | μm | 0 | NR | NR | 0 | NR | 2 | NR | 0 |

| (particles/mL) | | | | | | | | | |
|---|---|---|---|---|---|---|---|---|---|
| Total Protein Content by | | 158.2 | 159.1 | 157.5 | 158.3 | 152.3 | 161.9 | 152.2 | 159.1 |
| RP-UPLC (mg/mL) | | | | | | | | | |
| Purity by | Non-reduced; | 92.2 | NR | NR | 95.2 | NR | 94.9 | NR | 95.0 |
| MCE-SDS | % main peak | | | | | | | | |
| | Reduced; | 98.2 | NR | NR | 99.6 | NR | 97.6 | NR | 98.3 |
| | % heavy + | | | | | | | | |
| | light chain | | | | | | | | |
| Purity by | % HMW | 1.4 | 1.3 | 1.4 | 1.5 | 1.5 | 1.6 | 1.6 | 1.7 |
| SE-UPLC | % Main | 98.6 | 98.6 | 98.6 | 98.5 | 98.4 | 98.4 | 98.3 | 98.2 |
| | % LMW | 0.0 | 0.0 | 0.1 | 0.1 | 0.1 | 0.1 | 0.1 | 0.1 |
| Charge | % Region 1 | 29.1 | 28.2 | 29.2 | 29.8 | 30.5 | 30.5 | 30.3 | 29.6 |
| Variant | % Region 2 | 47.1 | 46.6 | 46.7 | 48.0 | 50.5 | 48.5 | 46.4 | 45.8 |
| Analysis by | % Region 3 | 23.8 | 25.2 | 24.0 | 22.3 | 19.0 | 21.0 | 23.3 | 24.6 |
| CEX-UPLC | | | | | | | | | |
| Charge | % Region 1 | 36.4 | NR | NR | 38.5 | NR | 38.8 | NR | 37.0 |
| Variant | % Region 2 | 50.4 | NR | NR | 49.0 | NR | 47.8 | NR | 50.5 |
| Analysis | % Region 3 | 13.2 | NR | NR | 12.5 | NR | 13.4 | NR | 12.5 |
| by iCIEF | | | | | | | | | |

| | | | | | | | | |
|---|---|---|---|---|---|---|---|---|
| % Relative Potency (Bioassay) | 90 | NR | NR | 97 | NR | 109 | NR | 106 |

Formulated drug substance was stored for extended periods of time (up to 36 months) at −20° C., and drug product was stored for extended periods of time (up to 24 months) at 5° C. As seen in Tables 13 and 14, formulated H4H1276S showed stability, e.g., values remained within acceptable ranges in all tests, over the entire range of storage lengths.

### Example 13: Containers

The primary container for antibody drug product intended for clinical development and product commercialization is a pre-filled syringe, which is presented as either a stand-alone syringe for self-injection or incorporated into an auto injector device for self-administration. The antibody formulations can also be developed in glass vials (for delivery by intravenous infusion).

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

What is claimed is:

1. A liquid pharmaceutical formulation comprising: (a) an antibody or antigen-binding fragment that binds specifically to human angiopoietin-like protein 3 (ANGPTL3); (b) a buffer; (c) an organic cosolvent; and (d) at least one viscosity modifier, wherein the formulation has a pH of 6.0±0.3.

2. The pharmaceutical formulation of claim 1, wherein the formulation further comprises at least one amino acid.

3. The pharmaceutical formulation of claim 1, wherein the antibody or antigen-binding fragment comprises three heavy chain complementarity determining regions (HCDR1, HCDR2, HCDR3) contained within a heavy chain variable region (HCVR) having an amino acid sequence SEQ ID NO: 66, and three light chain complementarity determining region (LCDR1, LCDR2, LCDR3) contained within a light chain variable region (LCVR) having an amino acid sequence SEQ ID NO: 74.

4. The pharmaceutical formulation of claim 1, wherein the antibody or antigen-binding fragment comprises a heavy chain variable region (HCVR) of SEQ ID NO: 66 and a light chain variable region (LCVR) of SEQ ID NO: 74.

5. The pharmaceutical formulation of claim 1, wherein the antibody or antigen-binding fragment comprises a HCDR1/HCDR2/HCDR3/LCDR1/LCDR2/LCDR3 sequence combination SEQ ID NO: 68/70/72/76/KAS/80.

6. The pharmaceutical formulation of claim 1, wherein the antibody concentration is from 5 mg/mL±0.75 mg/ml to 250 mg/mL±37.5 mg/mL.

7. The pharmaceutical formulation of claim 6, wherein the antibody concentration is 150 mg/ml±22.5 mg/mL.

8. The pharmaceutical formulation of claim 6, wherein the antibody concentration is 175 mg/mL±26.25 mg/mL.

9. The pharmaceutical formulation of claim 1, wherein the buffer is histidine.

10. The pharmaceutical formulation of claim 9, wherein the histidine concentration is from 5 mM±1 mM to 20 mM±4 mM.

11. The pharmaceutical formulation of claim 10, wherein the histidine concentration is about 10 mM±2 mM.

12. The pharmaceutical formulation of claim 1, wherein the organic cosolvent is polysorbate.

13. The pharmaceutical formulation of claim 12, wherein the polysorbate concentration is from 0.01% w/v±0.005% to 0.5% w/v±0.25%.

14. The pharmaceutical formulation of claim 13, wherein the polysorbate concentration is 0.1% w/v±0.05%.

15. The pharmaceutical formulation claim 12, wherein the organic cosolvent is polysorbate 80.

16. The pharmaceutical formulation of claim 1, wherein the at least one viscosity modifier is selected from the group consisting of arginine-HCl, sodium chloride, histidine-HCl, sodium acetate, calcium chloride, magnesium chloride, calcium acetate, and magnesium acetate.

17. The pharmaceutical formulation of claim 16, wherein the at least one viscosity modifier is arginine HCl.

18. The pharmaceutical formulation of claim 17, wherein the arginine HCl concentration is concentration is from about 50 mM to about 75 mM.

19. The pharmaceutical formulation of claim 18, wherein the arginine HCl concentration is about 70 mM.

20. The pharmaceutical formulation of claim 2, wherein the amino acid is proline.

21. The pharmaceutical formulation of claim 20, wherein the proline concentration is from 0 to 5%±1%.

22. The pharmaceutical formulation of claim 21, wherein the proline concentration is 3%±0.6%.

23. A liquid pharmaceutical formulation comprising: i) 150 mg/mL±22.25 mg/mL anti-ANGPT3 antibody or an antigen-binding fragment thereof; ii) from 5 mM±1 mM to 20 mM±4 mM histidine; iii) from 0.1% w/v±0.05% to 0.5% w/v±0.25% polysorbate 80; iv) from 50 mM to 75 mM arginine-HCL; and v) from 1%±0.2% to 5%±1% w/v proline, at pH 6.0±0.3.

24. The pharmaceutical formulation of claim 23, wherein the histidine concentration is 10 mM±2 mM, wherein polysorbate concentration is 0.1% w/v±0.05%, wherein the arginine-HCl concentration is about 70 mM, and wherein the proline concentration is 3% w/v±0.6% w/v.

25. The pharmaceutical formulation of claim 1, wherein the formulation has viscosity less than about 20 cP.

26. The pharmaceutical formulation of claim 1, wherein at least about 95% of the antibody has native conformation after 21 days at 45° C.

27. The pharmaceutical formulation of claim 1, wherein at least about 45% of the antibody is the main charge variant of the antibody after 21 days at 45° C.

28. The pharmaceutical formulation of claim 1, wherein at least about 98% of the antibody has native conformation after 36 months at 5° C.

29. The pharmaceutical formulation of claim 1, wherein at least about 55% of the antibody is the main charge variant of the antibody after 36 months at 5° C.

30. The pharmaceutical formulation of claim 1, wherein at least about 98% of the antibody has native conformation after 36 months at −30° C.

31. The pharmaceutical formulation of claim 1, wherein at least about 57% of the antibody is the main charge variant of the antibody after 36 months at −30° C.

32. The pharmaceutical formulation of claim 23, wherein the antibody or antigen-binding fragment comprises three heavy chain complementarity determining regions (HCDR1, HCDR2, HCDR3) contained within a heavy chain variable region (HCVR) having an amino acid sequence SEQ ID NO:66, and three light chain complementarity determining region (LCDR1, LCDR2, LCDR3) contained within a light chain variable region (LCVR) having an amino acid sequence SEQ ID NO: 74.

33. The pharmaceutical formulation of claim 23, wherein the antibody or antigen-binding fragment comprises a heavy chain variable region (HCVR) of SEQ ID NO: 66 and a light chain variable region (LCVR) of SEQ ID NO: 74.

34. The pharmaceutical formulation of claim 23, wherein the antibody or antigen-binding fragment comprises a HCDR1/HCDR2/HCDR3/LCDR1/LCDR2/LCDR3 sequence combination SEQ ID NO: 68/70/72/76/KAS/80.

35. The pharmaceutical formulation of claim 1, wherein the formulation is in a pre-filled syringe or autoinjector.

36. The pharmaceutical formulation of claim 1, wherein the formulation is in a glass vial.

37. A kit comprising a pharmaceutical formulation of claim 1, a container, and instructions.

38. The kit of claim 37, wherein the container is a prefilled syringe or autoinjector.

39. A method for treating, preventing, or ameliorating any disease or disorder associated with ANGPTL3 activity or mediated by ANGPTL3 in a subject, comprising administering to the subject the pharmaceutical formulation of claim 1.

40. The method of claim 39, wherein the pharmaceutical formulation is administered subcutaneously to the subject.
